(19) United States
(12) Patent Application Publication
(10) Pub. No.: US 2025/0339391 A1
(43) Pub. Date: Nov. 6, 2025

(54) METHODS FOR EFFECTIVE MANAGEMENT OF PAIN

(71) Applicants: South Rampart Pharma, Inc., New Orleans, LA (US); The Board of Supervisors of Louisiana State University and Agriculture and Mechanical College, Baton Rouge, LA (US)

(72) Inventors: Hernan A. Bazan, New Orleans, LA (US); Nicolas Bazan, New Orleans, LA (US)

(73) Assignees: South Rampart Pharma, Inc., New Orleans, LA (US); The Board of Supervisors of Louisiana State University and Agriculture and Mechanical College, Baton Rouge, LA (US)

(21) Appl. No.: 19/181,999

(22) Filed: Apr. 17, 2025

Related U.S. Application Data
(60) Provisional application No. 63/636,022, filed on Apr. 18, 2024.

(51) Int. Cl. A61K 31/18; A61P 29/00

(52) U.S. Cl. CPC A61K 31/18; A61P 29/00

(57) ABSTRACT

The present disclosure provides methods for ameliorating a pain in a subject in need thereof comprising administering a therapeutic amount of a composition comprising SRP-001 or a pharmaceutically acceptable salt thereof, wherein SRP-001 induces analgesia by generating N-arachidonoylphenolamine (AM404) in the midbrain periaqueductal gray (PAG) of the subject, and thereby ameliorates the pain. SRP-001 is a non-opioid and non-hepatotoxic small molecule that does not produce the hepatotoxic metabolite N-acetyl-p-benzoquinone-imine (NAPQI) and preserves hepatic tight junction integrity at high doses.

### CROSS-REFERENCES TO RELATED APPLICATIONS

This application claims priority to U.S. Provisional Patent Application No. 63/636,022, filed Apr. 18, 2024, the content of which is hereby incorporated by reference in its entirety for all purposes.

### GOVERNMENT SUPPORT CLAUSE

This invention was made with Government support under R42 NS 119103 awarded by the National Institutes of Health. The Government has certain rights in the invention.

### BACKGROUND

Worldwide, pain affects 27% of adults, and given its high prevalence and disability sequelae, it is a global health burden. In the United States (U.S.), pain affects more adults than diabetes and cancer combined, with an estimated cost of $635 billion/year to the healthcare system. Current medications are either addictive (e.g., opioids) or cause harm to the liver (e.g., acetaminophen/paracetamol or ApAP) or kidney (e.g., non-steroidal anti-inflammatory drugs; NSAIDs). Although ApAP is an effective pain reliever in various acute and chronic pain conditions, its narrow therapeutic index due to the risk of hepatotoxicity limits its clinical utility. While ApAP is commonly used for mild to moderate pain relief, it is not generally considered as effective as opioids for treating severe pain conditions. Additionally, it's important to note that the efficacy of ApAP in managing chronic pain is limited, and its use is often constrained by its hepatotoxicity. ApAP hepatotoxicity remains the most common cause of acute liver failure in the U.S. and the United Kingdom (U.K.). Annually, ˜30,000 patients are hospitalized for ApAP hepatotoxicity in the U.S., and inadvertent hepatotoxicity is the etiology in half of the cases. Although most patients experience only mild acute liver injury, such as a transient increase in liver transaminase release resulting in hepatitis and cholestasis, acute liver failure ensues in untreated patients who have ingested large doses. Some acute fulminant hepatic failure patients progress to convulsions, coma, and death. Though the proportion of liver transplants due to ApAP overdose varies depending on the country, it is generally acknowledged that ApAP overdose is a significant cause of acute fulminant hepatic failure leading to liver transplantation. Notably, up to 20% of liver transplants in some centers are due to ApAP-associated liver failure.

In response, some countries have implemented restrictions on the sale and availability of ApAP to reduce the risk of accidental and intentional overdoses. Australian regulators recently considered an outright ban on ApAP due to these concerns. However, they ultimately elected to implement restrictions on the maximum number of tablets per package and encourage retailers to limit the number of packages consumers can purchase. In 1998, the U.K. introduced regulations to limit the sale of ApAP in non-pharmacy retail outlets to packs containing a maximum of 16 tablets. In Canada, the risks of ApAP toxicity persisted despite labeling changes implemented in 2009 and 2016 to communicate the risks of ApAP overdose and promote safe use; monthly rates of hospital and intensive care unit admissions for accidental ApAP overdose were unchanged from April 2004 and March 2020.

Because of ApAP's hepatotoxicity, considerable efforts have been devoted to designing safer analgesic and antipyretic analogs, including a recent triazole bioisostere. However, none have proven effective beyond the pre-clinical phase. ApAP hepatotoxicity is associated with forming the electrophilic metabolite, N-acetyl-p-benzoquinoneimine (NAPQI), through an oxidative process mediated by CYP2E1 and CYP3A isoforms of CYP450. NAPQI is normally neutralized by a glutathione (GSH)-mediated Phase II metabolic pathway and eliminated as a mercapturic acid. During overuse, the conjugative Phase II metabolism becomes saturated, leading to GSH depletion and accumulation of NAPQI that react with nucleophilic macromolecules, triggering events that result in hepatotoxicity and hepatocellular death, referred to as acetaminophen-induced liver injury (AILI).

To overcome the foregoing limitations, the present disclosure provides a non-hepatotoxic analog of ApAP, which is SRP-001 for the treatment of pain. SRP-001 is an efficacious, non-opioid, non-hepatotoxic small molecule effective for pain treatment.

### BRIEF SUMMARY

SRP-001 was developed as a non-opioid and non-hepatotoxic small molecule that, unlike ApAP, does not produce the hepatotoxic metabolite N-acetyl-p-benzoquinone-imine (NAPQI) and preserves hepatic tight junction integrity at high doses.

As such, in one embodiment, the present disclosure provides a method for ameliorating a pain in a subject in need thereof, the method comprising:
administering a therapeutic amount of a composition comprising SRP-001 or a pharmaceutically acceptable salt thereof, wherein SRP-001 induces analgesia by generating N-arachidonoylphenolamine (AM404) in the midbrain periaqueductal gray (PAG) of the subject, to thereby ameliorate the pain.

In certain aspects, the pain is categorized as acute, chronic, or neuropathic pain.

In certain aspects, the therapeutically effective amount comprises a dose of about 10 μM to about 10 mM of the composition is administered to the subject.

In certain aspects, the composition is administered in a single dose.

In certain aspects, the composition is administered at intervals of about 4 hours, 12 hours, or 24 hours.

In certain aspects, the composition is administered orally, parentally, transdermally, topically, or nasally.

In certain aspects, the composition further comprises one or more pharmaceutically acceptable excipients, carriers, or a combination thereof.

In certain aspects, the SRP-001 is present in an amount selected from the group consisting of from about 99.5% to about 0.001%, from about 95% to about 0.1%, and from about 90% to about 0.5%, by weight, based on the total combined weight of the SRP-100 and at least one wetting agent, not including other excipients.

In certain aspects, at least one wetting agent is present in an amount selected from the group consisting of from about 0.01% to about 99.5% by weight, from about 0.1% to about 95% by weight, and from about 0.5% to about 90% by weight, and from about 0.5% to about 2%, based on the total combined dry weight of SRP-100 and at least one wetting agent, not including other excipients.

In certain aspects, the at least one wetting agent is present in an amount selected from the group consisting of from about less than 10%, less than 5%, less than 2% and less than 1%, based on the weight of the nanoparticulate composition.

In certain aspects, at least one wetting agent is selected from the group consisting of a bile salt and an alkali salt of a bile acid.

In certain aspects, the bile salt is selected from the group consisting of sodium, potassium, lithium, calcium, arginine, lysine and the ammonium salt of a bile acid.

In certain aspects, the salt of a bile acid is selected from the group consisting of cholic acid, glycocholic acid, taurocholic acid, deoxycholic acid, glyco- or taurodeoxycholic acid, chenodeoxycholic acid and glyco- or taurochenoxydeoxycholic acid.

In certain aspects, the method further comprising a secondary wetting agent selected from the group consisting of polyvinyl alcohol, polyvinylpyrrolidone, polyethylene glycol, and cellulose derivatives.

In certain aspects, the composition is administered in the form of a pill, tablet, capsule, cream, spray, lotion, a suspension or aqueous solution.

In certain aspects, the composition is a nanoparticulate particle having an average particle size of less than about 500 nm or less than about 300 nm.

In certain aspects, the composition exhibits analgesia, antipyresis, or a combination thereof

In certain aspects, the composition reduces the risk of hepatotoxicity by at least about 20% compared to acetaminophen (ApAP).

In certain aspects, the administration does not generate the hepatotoxic metabolite NAPQI.

In certain aspects, the specific neuronal target of the composition is one or more of CB1, FAAH or TRPV1, which underlies the analgesia.

In certain aspects, SRP-001 has a half-life in said subject of about 4.9 to about 9.8 hours.

In certain aspects, direct comparison of SRP-001 vs. ApAP at equimolar doses reveals no mortality for SRP-001, whereas ApAP shows a dose-dependent increase in mortality.

These and other aspects, objects and embodiments will become more apparent when read with the detailed description and figures that follow.

### BRIEF DESCRIPTION OF THE DRAWINGS

FIG. 1A-FIG. 1O show SRP-001's absent hepatotoxicity is due to lack of NAPQI formation and maintenance of hepatic tight junction integrity. FIG. 1A shows possible oxidation of SRP-001 to the corresponding N-acyl-p-benzoquinone imine by CYP enzymes (CYP P450 2E1 and 3A4). FIG. 1B shows ApAP is metabolized by oxidation into NAPQI by CYP P450 2E1 and 3A4.c, Doses known to be hepatotoxic for ApAP (600 mg/kg) but not SRP-001 (600 mg/kg) demonstrates centrilobular hepatic necrosis in nitrotyrosine-labeled hepatic sections from CD1 mice (100×, c, first column), [(n=5) mice per treatment group]. Hepatic tight junctions (ZO-1 label [3D-stacking, 1000×; scale bar=5 μm] FIG. 1C shows second column) present between hepatocytes reveal a clear “chicken wire” configuration (vehicle) and are disrupted with toxic doses of ApAP but not SRP-001. FIG. 1D shows quantification of the hepatic tight junctions by ZO-1 staining. There is a marked decrease in ZO-1 in ApAP-treated liver sections compared to vehicle and SRP-001, wherein ZO-1 staining is preserved. FIG. 1E shows Kaplan-Meier survival curves comparing equimolar doses of ApAP (150 to 900 mg/kg) and SRP-001 (402 to 2,414 mg/kg) demonstrate a dose-dependent increase in mortality by 72 hours for ApAP-treated groups (1/10 at 1,984 mM/kg, 3/10 at 3,969 mM/kg, and 7/10 at 5,954 mM/kg), with no mortality observed in the SRP-001 and placebo cohorts; n=10 per group (CD-1 male mice). FIG. 1F shows ApAP exposure but not SRP-001 led to an increase in the liver transaminases and 75% (15 out of 20) of ApAP treated animals were dead in 48 hours and none in SRP-001 treated animals (data not shown). FIG. 1G shows ApAP-treated but not SRP-001-treated mice produce the hepatotoxic metabolite NAPQI. Serum NAPQI peak is shown at 5.1 min on the chromatogram. NAPQI peak appears after IP-injection with ApAP but not SRP-001 (600 mg/kg). FIG. 1H shows ApAP-TP sample with a standard NAPQI spike demonstrates the same chromatographic retention time. FIG. 1I shows full fragmentation pattern of the NAPQI standard and its likely fragments. FIG. 1J shows the full fragmentation spectrum from IP-ApAP sample's NAPQI peak. Note: The spectrum matches well the standard in FIG. 1I. FIG. 1K-FIG. 1N show LC-MS/MS retention time peak at 5.63 demonstrates SRP-001 (FIG. 1K) as it matches the full fragmentation pattern of SRP-001 from an ip-injected SRP-001 animal showing its fragmentation peaks (FIG. 1M); similarly, retention time 5.76 demonstrates the benzoquinoimine produced by SRP-001 (FIG. 1L); and (FIG. 1N) the resulting fragmentation pattern of the N-acyl-p-benzoquinone imine of SRP-001. FIG. 1O shows structures matching full fragmentation pattern peaks of SRP-001 and its predicted benzoquinoimine.

FIG. 2A-FIG. 2N show both ApAP and SRP-001 induce analgesia in the von Frey hyperalgesia in vivo assay and are antipyretic. FIG. 2A shows a timeline outlining the experimental design for the von Frey with electronic detection hyperalgesia/allodynia in vivo assay. FIG. 2B-FIG. 2I show two separate doses of ApAP and SRP-001—32 and 100 mg/kg body weight—were tested using young male rats in von Frey. The threshold for paw withdrawal increased from 18 g to 40 g and subsequently to 55 g in SRP-001-treated animals; it is more efficacious than ApAP at similar doses. (n=10) rats for ApAP, and (n=20) rats for SRP-001. Note: The left hind paw is treated with either CFA or saline, and the right hind paw is an internal control and is not injected. j, SRP-001 has a more potent antinociceptive effect compared to ApAP at equimolar doses (μmol/kg). ED₅₀ curves for SRP-001 are shifted to the left compared to ApAP in the hyperalgesia/allodynia (von Frey) and in a visceral pain model (acetic acid writing assay); FIG. 2K shows timeline showing experimental design for antipyresis using LPS from Escherichia coli (100 μg/kg, 0111:B4). FIG. 2L shows there are no significant changes in body temperature of mice injected with 0.9% saline (vehicle) throughout the course of the experiment (n=10) FIG. 2M-FIG. 2N shows SRP-001 and ApAP, respectively have comparable antipyresis. (n=20) mice.

FIG. 3A-FIG. 3K show the antinociception/analgesia is mediated via AM404 production in the periaqueductal gray (PAG) region of the brain and human Single and Multiple Ascending Doses (SAD and MAD). Phase 1 clinical trial reveals a favorable pharmacokinetic profile. FIG. 3A shows detection of deuterated (D₁₀-SRP-001) in the rat brain 30 min after IP injection. The full fragmentation spectrum of D₁₀-SRP-001 detected in the brain matches FIG. 3B shows the standard SRP-001 compound spectrum. FIG. 3C shows the structure of D₁₀-SRP-001 shows fragmentation patterns of D₁₀-SRP-001 that match the spectra shown in the brain and with the deuterated standard. FIG. 3D shows the MRM (m/z 416→186) for D₁₀-SRP-001 shows the elution time at ˜2.2 min. FIG. 3E shows quantification of AM404 production in the CNS periaqueductal gray region. FIG. 3F shows AM404 is expressed in the periaqueductal gray area of the brain following SRP-001 IP injection. LC-MS/MS detection of AM404 in the rat periaqueductal gray area after SRP-001 (top panel) and ApAP (bottom panel) IP injections. The AM404 structure and major fragments. FIG. 3G shows the peaks are confirmed to be AM404 by co-spiking AM404 standard to the samples. The major peaks detected by LC-MS/MS demonstrate that the peak 9.9 min from LC is the AM404 compound. These peaks align to the FIG. 3E AM404 fragments. FIG. 3H shows design of the first-in-human Phase 1 clinical trial for SRP-001. A randomized, double-blind, placebo-controlled study to assess the safety, tolerability, and pharmacokinetics of single and multiple ascending oral doses of SRP-001 and to characterize the effect of food on the pharmacokinetics of SRP-001 in healthy male and female subjects. Single ascending dose (SAD) escalation from 300 mg to 2,000 mg (fasted state) and 900 mg (fed state). FIG. 3I and FIG. 3J show geometric mean SRP-001 plasma concentration-time profiles following oral administration (logarithmic plot) show a proportional or super-proportional increase in Cₘₐₓ (peak time to concentration at 1 h post-dose) with a mono- or bi-phasic concentration decline in the SAD (i) and the MAD cohorts with an arithmetic mean T_{1/2} of 5.57 h and a geometric mean T_{1/2} of 4.92 h. FIG. 3K show SRP-001 plasma concentration-time profiles at Day 5.

FIG. 4A-FIG. 4K show single-cell transcriptomics of midbrain periaqueductal gray (PAG) cells in CFA-induced chronic inflammatory pain model. FIG. 4A shows an overview of single-cell RNA sequencing (scRNA-seq) experimental design to define the mechanism of action (MOA) of ApAP and SRP-001. The periaqueductal gray (PAG) midbrain region was dissected to isolate cell nuclei for sequencing library generation using the 10× Genomics Chromium platform. Single cell data was embedded after dimensionality reduction using uniform manifold approximation and projection (UMAP). Cell clusters were annotated, followed by differential gene expression analysis. FIG. 4B shows UMAP plots showing the distribution of annotated cell clusters color-coded by each cluster—astrocytes, excitatory neurons, inhibitory interneurons, oligodendrocytes, oligodendrocyte precursor cells (OPC), and microglia, from the aggregated clusters of all the 4 samples. FIG. 4C shows UMAP plots showing the distribution of annotated cell clusters generated by the workflow of Seurat across 4 different samples—Vehicle, CFA_Veh, CFA_ApAP, and CFA_SRP-001, respectively. FIG. 4D-FIG. 4I show marker gene feature plots showing selected distinctive marker genes used for cell annotation of single cell clusters into 6 different cellular subpopulations. FIG. 4D and FIG. 4E show oligodendrocytes; FIG. 4F shows astrocytes; FIG. 4G shows OPC; FIG. 4H shows Glutamatergic neurons and FIG. 4I shows GABAergic neurons. FIG. 4J and FIG. 4K show GO enrichment analysis results for top 50 differentially expressed genes between CFA_Vehicle and CFA_ApAP, and between CFA_Vehicle and CFA_SRP-001-gene concept network showing the linkage between the DE genes and GO terms, and Barplot of the enriched GO terms from the selected genes.

FIG. 5A-FIG. 5G show cell annotation, marker gene feature plots, and endocannabinoid signaling pathway from neurons of PAG region cells in CFA-induced chronic inflammatory pain model with ApAP and SRP-001 treatments. FIG. 5A shows a heatmap of top 40 canonical pathways for neuronal clusters across samples—CFA_Veh, CFA_ApAP, and CFA_SRP-001 with the comparisons between them as follows—Vehicle vs CFA_Vehicle, CFA_Vehicle vs CFA_ApAP, and CFA_ApAP vs CFA_SRP-001. The heatmap shows that when CFA_Vehicle is compared to Vehicle, there is upregulation of several genes that are predicted to affect cellular signaling related to these pathways highlighted in the heatmap, and when CFA_ApAP and CFA_SRP-001 are compared to CFA_Vehicle, there is downregulation of those upregulated pathways in both cases, with more downregulation for CFA_SRP-001 than CFA_ApAP. FIG. 5B-FIG. 5C show CellChat computed cell-cell interaction network for CFA_ApAP and CFA_SRP-001 group datasets showing number and strength of interactions describing similarities between the two groups. FIG. 5D-FIG. 5E shows outgoing communication patterns in individual secreting cell types for CFA_ApAP and CFA_SRP-001. FIG. 5F shows heatmap of DESeq2-normalized expression values from scRNA-seq data for genes involved in endocannabinoid signaling genes. FIG. 5G shows endocannabinoid signaling pathways generated by Qiagen Ingenuity Pathway Analysis (IPA) with an overlay based on DESeq2-normalized expression values of differentially expressed genes (DEGs) in neuronal clusters CFA_Vehicle vs CFA_SRP-001 treatments, respectively.

FIG. 6 shows ApAP metabolic pathways. There is pronounced hepatotoxicity when NAPQI accumulation overwhelms glutathione conjugation.

FIG. 7A-FIG. 7D show SRP-001 is non-hepatotoxic and non-cardiotoxic. FIG. 7A shows histograms demonstrate that the serum NAPQI levels are significantly enhanced only for the ApAP-injected animals and not for vehicle- or SRP-001-injected animals. The significant rise in NAPQI levels for APAP-injected animals is regardless of the administration route (for both IP and per os). Hence, SRP-001 is considered non-hepatotoxic with the lack of production of NAPQI as compared to ApAP. FIG. 7B shows TUNEL staining shows prominent TUNEL positive nuclei, indicating more apoptotic cells in ApAP dosed animals (600 mg/kg), while vehicle and SRP-001 dosed animals (600 mg/kg) have almost no TUNEL signal. FIG. 7C shows quantification of TUNEL staining from liver sections from mice dosed with either vehicle or ApAP or SRP-001 (n=15) (p<0.0001). FIG. 7D shows SRP-001 is considered non-cardiotoxic as evidenced by an absent relevant signal in an in vitro hERG assay in human embryonic kidney cells.

FIG. 8A-FIG. 8P show Von Frey for young female and aged male rats. FIG. 8A-FIG. 8H show Von-Frey data of young female rats. FIG. 8I-FIG. 8P show Von Frey data of aged male rats. For the cohort of young female and aged male rats, two separate doses of ApAP and SRP-001—32 and 100 mg/kg body weight—were tested, respectively. SRP-001 oral nanosuspension and ApAP showed comparable analgesic activity for both animal cohorts (a, i) For SRP-001-treated animals, in the CFA-injected left hind paw, the threshold for paw withdrawal increased from 25 g to 32 g and subsequently to 40 g for (32 mg/kg and 100 mg/kg) dose, respectively. (n=20) rats for each treatment group for both cohorts.

FIG. 9A-FIG. 9K show Tail Flick (somatic pain) and Acetic acid writhing (visceral pain) assays. FIG. 9A shows a timeline showing the experimental design of the cold tail-flick assay. FIG. 9B and FIG. 9C show cold tail-flick assay of young (male and female). FIG. 9D shows aged (male) mice. At the high dose (100 mg/kg) of ApAP or SRP-001, there is significant analgesia in all experimental cohorts. (n=10) mice for young male and aged male experimental groups and (n=20) or young female mice. FIG. 9E shows a timeline showing the experimental design of the acetic acid-induced writhing assay of aged (male) and young (male and female) mice. Experimental groups: young female (FIG. 9F and FIG. 9I), young male (FIG. 9G and FIG. 9J), and aged male (FIG. 9H and FIG. 9K) for ApAP and SRP-001 respectively. At the high dose (100 mg/kg) of ApAP or SRP-001, there is significant analgesia in all experimental cohorts. (n=10) mice per treatment group for aged male mice, (n=20) mice for vehicle, and (n=10) per treatment group for young male and young female.

FIG. 10A-FIG. 10I show quimolar dose-response curves (μmol/kg) for Von Frey, tail flick, and acetic acid writhing assays. Dose-response curves of von Frey (FIG. 10A-FIG. 10C, hyperalgesia), tail flick (FIG. 10D-FIG. 10F, somatic pain), and acetic acid writhing/abdominal contractions (FIG. 10G-FIG. 10I, visceral pain) assays for young male and female animals and aged male animals. All the different analgesic assays and resulting dose-response curves show a clear shift to the left of the SRP-001 curve compared to the dose-response curve of ApAP with similar equimolar doses of both drugs.

FIG. 11A-FIG. 11Q show Hargreaves assay for young male and young female rats. FIG. 11A shows timeline showing the experimental design of the Hargreaves assay for young female, young male, and for aged male rats. FIG. 11B-FIG. 11I, Paw withdrawal latencies for young male rats and FIG. 11J-FIG. 11Q, for young female rats. At the high dose (100 mg/kg) of ApAP or SRP-001, there is significant analgesia in all experimental cohorts. (n=20) rats for each treatment group.

FIG. 12A-FIG. 12H show Hargreaves assay for aged male rats. FIG. 12A-FIG. 12H show paw withdrawal latencies for aged male rats. At the high dose (100 mg/kg) of ApAP or SRP-001, there is significant analgesia in all experimental cohorts. (n=20) rats for each treatment group.

FIG. 13A-FIG. 13D show antipyresis-yeast. FIG. 13A shows timeline for experimental design of fever induction by yeast derived from Saccharomyces cerevisiae Type II, and subsequent administration of either ApAP or SRP-001 at 75 mg/kg body weight. FIG. 13B shows no significant changes in body temperature of mice injected with 0.9% saline (vehicle) (Sham) throughout the course of the experiment. FIG. 13C and FIG. 13D show after yeast injection, there is significant fever induction, followed by a subsequent reduction in core body temperature with the per os administration of either ApAP or SRP-001. (n=10) mice per treatment group.

FIG. 14A-FIG. 14J show PCA Dimension Plots—Single cell heatmaps showing the top 30 differentially expressed genes in each cluster for the first 10 principal clusters (PCs) selected based on the elbow plot. FIG. 14A-FIG. 14J show PC_1 through PC_10, respectively.

FIG. 15A-FIG. 15D show Highly Variable Gene (HVG) plots for each sample show top 10 HVGs across all cell clusters for each sample—Vehicle, CFA_Vehicle, CFA_ApAP, and CFA_SRP-001.

FIG. 16A-FIG. 16C show Heat Map of top 50 differentially expressed genes from DE analysis using Seurat for all samples showing comparison between FIG. 16A shows Vehicle and CFA_Vehicle, FIG. 16B shows CFA_Vehicle vs CFA_ApAP and FIG. 16C shows CFA_Vehicle vs CFA_SRP-001.

FIG. 17A-FIG. 17F show dot plots showing differential expression of pain-related genes—Ion Channels across treatment groups—Saline (Vehicle), CFA_Vehicle, CFA_ApAP, and CFA_SRP-001, in different cell clusters—FIG. 17A shows Astrocytes, FIG. 17B shows Endothelial cells, FIG. 17C shows Neurons, FIG. 17D shows OPCs, FIG. 17E shows Oligodendrocytes, and FIG. 17F shows Microglia. The size of the dots represents the percentage of barcodes within a cluster and the color corresponds to the average expression (scaled data) within a cluster for each gene shown.

FIG. 18A-18D show dot plots showing differential expression of pain-related genes—Transcription factors across treatment groups—Saline (Vehicle), CFA_Vehicle, CFA_ApAP, and CFA_SRP-001, in different cell clusters—FIG. 18A shows OPCs, FIG. 18B shows Oligodendrocytes, FIG. 18C shows Neurons, and FIG. 18D shows Astrocytes.

FIG. 19A-FIG. 19D show dot plots showing differential expression of pain-related genes—GPCRs across treatment groups—Saline (Vehicle), CFA_Vehicle, CFA_ApAP, and CFA_SRP-001, in different cell clusters—FIG. 19A shows Astrocytes, FIG. 19B shows Microglia, FIG. 19C shows Neurons, and FIG. 19D shows OPCs.

FIG. 20A-FIG. 20B show regulation in Vehicle vs CFA_Vehicle and CFA_Vehicle vs CFA_ApAP or CFA_Vehicle vs CFA_SRP-001 comparisons. Pathway overlay indicates decreased or increased expression measured experimentally respectively; or the overlay indicates predicted molecular relationships based on dataset measurements of genes known to interact/effect the predicted molecule/function. FIG. 20A shows expression and prediction overlay from DSeq2 comparison values between CFA_Vehicle vs CFA_ApAP. FIG. 20B shows expression and prediction overlay from DSeq2 comparison values between CFA_Vehicle vs CFA_SRP-001.

FIG. 21A-FIG. 21B show Gene Network Expression for mechanical nociception and FAAH pathways generated by Ingenuity Pathway Analysis (IPA). FIG. 21A shows Heatmap of DSeq2 computed expression values from scRNAseq data involved in genes pertaining to mechanical nociception. Similarities in the gene modulatory mechanism of ApAP and SRP-001 are highlighted by the shared trend in expression values of the genes shown. FIG. 21B shows Heatmap of DSeq2 computed expression values for FAAH related genes. Similarities in the gene modulatory mechanism of ApAP and SRP-001 are highlighted by the shared trend in expression values of the genes shown.

FIG. 22A-FIG. 22D show Heat Maps of Canonical Pathways for different cell clusters. FIG. 22A shows Oligodendrocytes, FIG. 22B shows Interneurons, FIG. 22C shows Microglia, FIG. 22D shows Astrocytes across the 4 sample groups—Saline (Vehicle), CFA_Vehicle, CFA_ApAP and CFA_SRP-001 with the comparisons—CFA_Vehicle vs Vehicle, CFA_Vehicle vs CFA_ApAP and CFA_Vehicle vs CFA_SRP-001 treatment groups.

FIG. 23A-FIG. 23C show GO enrichment analysis results for top 50 differentially expressed genes between Vehicle and CFA_Vehicle. FIG. 23A shows Barplot of the enriched GO terms from the selected genes FIG. 23B shows enrichment map of the GO terms showing linkage between the terms based on overlapping gene sets. FIG. 23C shows gene concept network showing the linkage between the DE genes and GO terms.

FIG. 24 shows toxicokinetic (TK) beagle dog data. Following IV & PO Dosing in beagle dogs, SRP-001's uptake is rapid with Cmax in 15-30 min, and there is a dose-dependent response. Furthermore, SRP-001 is considered non-cardiotoxic, as evidenced by 1) a lack of findings in 28-day studies in beagle dogs or rats and 2) a normal telemetered safety pharmacology study in dogs (no prolonged QT_{c}; data not shown for brevity).

FIG. 25A-FIG. 25B show oral formulation: Jet milling nano-milling. Jet milling and nano-milling of SRP-001 for 17 h reduce particle size from 879.33 μm (FIG. 25B) to 0.1666 μm (FIG. 25A) and increase the solubility of SRP-001 to optimize for both oral (chronic pain) and IV (acute pain) formulations.

FIG. 26 shows Table 1: Summary of ED₅₀ values calculated from dose response curves generated by equimolar dosing (μmol/kg) of ApAP and SRP-001 in the different antinociceptive/analgesia assays.

FIG. 27 shows Table 2: PK by Subject (male Beagle dog) receiving SRP-001 oral nanosuspension dose up to 7 days.

FIG. 28 shows Table 2: PK by Subject (male Beagle dog) receiving SRP-001 oral nanosuspension dose up to 7 days.

### DETAILED DESCRIPTION

SRP-001 (also known as SRP-3D (DA)) exists as a compound of Formula I:

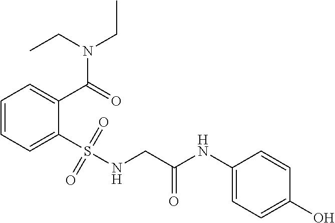 [SMILES: CCN(CC)C(=O)c1ccccc1S(=O)(=O)NCC(=O)Nc1ccc(O)cc1]

wherein the compound is substantially free of impurities. A polymorph of SRP-001 is described in WO 2024/191741, incorporated herein by reference. See too, U.S. Pat. No. 11,458,142, incorporated herein by reference.

In certain instances, the compound is formulated as a nanosuspension or nanoparticulate compositions containing nanoparticles, i.e., particles having at least one external dimension of less than about 1 μm, of a compound of formula I and one or more than one wetting agents. Suitable wetting agents can preferably be selected from known organic and inorganic pharmaceutical excipients. Such excipients may include various polymers, low molecular weight oligomers, natural products, and surfactants. Preferred wetting agents include nonionic, anionic, cationic and zwitterionic surfactants, hydrophilic polymers, and polyether-derivatized polysaccharides. A more preferred wetting agent is a bile salt.

In certain instances, a crystalline compound of Formula I is jet milled. Thereafter, the milled compound of Formula I is dispersed in a solution comprising 1% hydroxypropyl cellulose (1% HPC) and sterile water for injection and subjected to wet milling. The target particle size is approximately D90 0.15 μm. The disclosure provides using nanomilling and jet-milling techniques, to prepare an oral nanoparticle suspension containing 100 mg/mL SRP-001 in preserved aqueous 1% HPC. An oral nanoparticle suspension containing 100 mg/mL SRP-001 in aqueous 1% HPC preserved with 0.1% sodium benzoate was used.

In certain instances, the present disclosure provides a method of treating or alleviating pain in a subject by administering SRP-001.

In certain instances, the present disclosure provides for a method of treating or reducing fever in a subject by administering SRP-001.

In certain instances, the present disclosure provides a method of preventing pain in a subject, reducing the incidence of pain in a subject, delaying the development of pain in a subject, preventing the development of pain in a subject, and/or palliating pain in a subject by administering SRP-001.

Non-limiting examples of pain comprise acute pain, chronic pain, neuropathic pain, nociceptive pain, post-surgical pain, eye pain, dental pain, ophthalmic pain, arthritic pain, post- and/or traumatic pain, or a combination thereof.

In certain instances, the present disclosure provides a method comprises administering to a subject in need thereof a therapeutically effective amount of the analgesic compound or composition as described herein. For example, the therapeutically effective amount of the analgesic compound or composition administered to a subject can comprise a dose of about 10 μM to about 10 mM, or a dose of about 50 μM to about 1 mM.

In certain instances, the analgesic and antipyretic compound is administered to a subject a single dose, such as in a bolus. In other embodiments, the compound is administered at intervals of about 4 hours, 12 hours, or 24 hours. In still other embodiments, the compound is administered continuously, such as in a drip IV infusion.

In certain instances, the SRP-001 composition can be administered orally, such as in a pill, tablet, aqueous solution, or capsule; parenterally, such as in an intravenous or intramuscular injection; transdermally, such as in a cream, lotion, or patch; or nasally, such as in a spray. In other embodiments, the composition can be administered subcutaneously, intrapulmonary, topically, intravitreally, transmucosally, rectally, and intranasally administration.

In certain instances, the SRP-001 composition or analgesic/antipyretic compound as described herein can be administered to a subject together with a therapeutically effective amount of a second active ingredient, such as an opioid and/or NSAID. The second active ingredient can be administered prior to, concurrently with, or subsequent to the administration of the compound or composition described herein.

In certain instances, the disclosure provides a medical kit for the treatment of pain and/or fever. In embodiments, the kit comprises printed instructions for administering the compound to the subject afflicted with pain or fever and an analgesic or antipyretic compound or composition as described herein.

The compounds and formulations herein described are useful in treating acute, chronic, and/or neuropathic pain. Current medications, including opioids, acetaminophen/paracetamol, and nonsteroidal anti-inflammatory drugs (NSAIDs), offer limited relief and have significant drawbacks, such as addiction and organ toxicity. The opioid crisis has further complicated the landscape, with 8.7 million people aged 12 or older misusing prescription opioids in the U.S. alone. These forms of pain degrade the quality of life and pose a significant economic burden.

Embodiments can be used for the treatment or amelioration of pain, non-limiting examples of which comprise post-surgical, neuropathic, dental, ophthalmic, arthritic, acute, chronic, inflammatory, and/or traumatic pain.

In embodiments, compounds and compositions as described herein, can be used as the only pharmacologically active compound in the treatment of neuropathic pain without a second active agent, such as a GABA analogue, such as Gabapentin (Neurontin). In other embodiments, the compounds and compositions as described herein can be administered to a subject concurrently with and/or in combination with a second active ingredient, such as an opioid or a non-steroidal anti-inflammatory drug (NSAID). Opioid drugs work by binding to opioid receptors in the brain and spinal cord. Non-limiting examples of such opioids comprise codeine, fentanyl, hydrocodone, hydrocodone/ApAP, hydromorphone, meperidine, methadone, morphine, oxycodone, oxycodone and ApAP, oxycodone and naloxone. Nonsteroidal anti-inflammatory drugs (NSAIDs) block the COX enzymes and reduce prostaglandins throughout the body. As a consequence, ongoing inflammation, pain, and fever are reduced. Non-limiting examples of NSAIDs comprise aspirin, celecoxib, diclofenac, diflunisal, etodolac, ibuprofen, indomethacin, ketoprofen, ketorolac, nabumetone, naproxen, oxaprozin, piroxicam, salsalate, sulindac, and tolmetin.

SRP-001 as described herein, can be incorporated into pharmaceutical compositions suitable for administration. Such compositions can comprise a compound as described herein and a pharmaceutically acceptable carrier. Thus, in some embodiments, the compounds of the disclosure are present in a pharmaceutical composition.

For example, a pharmaceutical composition comprising a compound as described herein can be used for preventing and/or treating pain, such as a therapeutically effective amount of a compound of Formula I in admixture with a pharmaceutical acceptable carrier or excipient. For example, a therapeutically effective amount of a compound of Formula I can be administered to a subject so as to prevent or reduce the onset of pain or prevent the severity of pain from increasing.

“Treatment” can refer to an approach for obtaining beneficial or desired clinical results, for example improvement or alleviation of any aspect of pain, such as post-surgical, neuropathic, dental, ophthalmic, arthritic, acute, chronic, inflammatory, and/or traumatic pain. Beneficial or desired clinical results comprise, but are not limited to, one or more of the following: including lessening severity, alleviation of one or more symptoms associated with pain including any aspect of pain (such as shortening duration of pain, and/or reduction of pain sensitivity or sensation).

“Ameliorating” pain or one or more symptoms of pain can refer to a lessening or improvement of one or more symptoms of a pain as compared to not administering a composition as described herein. “Ameliorating” can also comprise shortening or reduction in duration of a symptom. For example, a therapeutically effective amount of a compound of Formula I can be administered to a subject afflicted with pain so as to ameliorate, or lessen, the pain.

The term “alleviate” or “alleviating” can refer to lightening, reducing or lessening the severity of a symptom, condition, or disorder. For example, a treatment, such as a compound of Formula I, that reduces the severity of pain in a subject can be said to alleviate pain. For example, a therapeutically effective amount of a compound of Formula I can be administered to a subject afflicted with pain, wherein the severity of the pain is lessened. It is understood that, in certain circumstances, a treatment can alleviate a symptom or condition without treating the underlying disorder. In certain aspects, this term can be synonymous with the language “palliative treatment.”

Embodiments can be used for reducing the incidence of or delaying pain, non-limiting examples of which comprise post-surgical, neuropathic, dental, ophthalmic, arthritic, acute, chronic, inflammatory, and/or traumatic pain. “Reducing incidence” of pain can refer to any of reducing severity (which can include reducing need for and/or amount of (e.g., exposure to) other drugs and/or therapies generally used for this conditions), duration, and/or frequency (including, for example, delaying or increasing time to pain in an individual). As is understood by those skilled in the art, individuals can vary in terms of their response to treatment, and, as such, for example, a “method of reducing incidence of pain in an individual” reflects administering compositions as described herein, based on a reasonable expectation that such administration can cause such a reduction in incidence in that particular individual.

“Delaying” the development of pain can refer to deferring, hindering, slowing, retarding, stabilizing, and/or postponing progression of pain. This delay can be of varying lengths of time, depending on the history of the disease and/or individuals being treated. As is evident to one skilled in the art, a sufficient or significant delay can, in effect, encompass prevention, in that the individual does not develop pain. A method that “delays” development of the symptom is a method that reduces the probability of developing the symptoms in a given time frame and/or reduces the extent of the symptoms in a given time frame, when compared to not using the method.

“Development” or “progression” of pain can refer to initial manifestations and/or ensuing progression of the disorder. Development of pain can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of pain includes initial onset and/or recurrence. For example, embodiments as described herein can be used to prevent the development of pain or prevent the progression of pain.

Embodiments can be used for palliating pain, non-limiting examples of which comprise post-surgical, neuropathic, dental, ophthalmic, arthritic, acute, chronic, inflammatory, and/or traumatic pain.

“Palliating” pain or one or more symptoms of pain can refer to lessening the extent of one or more undesirable clinical manifestations of pain in an individual or population of individuals treated with a composition as described herein.

Embodiments comprise administering to a subject an effective amount of a composition as described herein, for the treatment of pain.

An “effective amount”, “sufficient amount” or “therapeutically effective amount” can refer to an amount sufficient to effect beneficial or desired clinical results including alleviation or reduction in the pain sensation. For purposes of this disclosure, an effective amount of a composition as described herein, can comprise an amount sufficient to treat, ameliorate, reduce the intensity of or prevent pain of any sort, such as post-surgical, neuropathic, dental, ophthalmic, arthritic, acute, chronic, inflammatory, and/or traumatic pain. In some embodiments, an effective amount of compositions as described herein can modulate the sensitivity threshold to external stimuli to a level comparable to that observed in healthy subjects. In other embodiments, this level is not comparable to that observed in healthy subjects but is reduced compared to not receiving the therapy.

Specific compositions as described herein, can be administered to a subject by any suitable means, such as oral, intravenous, parenteral, subcutaneous, intrapulmonary, topical, intravitreal, dermal, transmucosal, rectal, and intranasal administration. Parenteral infusions include intramuscular, intravenous, intraarterial, or intraperitoneal administration. The compounds can also be administered transdermally, for example in the form of a slow-release subcutaneous implant or as a transdermal patch. They can also be administered by inhalation. Although direct oral administration may cause some loss of desired activity, for example pain relieving activity, the analgesics can be packaged in such a way to protect the active ingredient(s) from digestion by use of enteric coatings, capsules or other methods known in the art.

Controlled-release pharmaceutical products have a common goal of improving drug therapy over that achieved by their non-controlled counterparts. The use of an optimally designed controlled-release preparation in medical treatment is characterized by a minimum of drug substance being employed to cure or control the condition in a minimum amount of time.

Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

Solutions or suspensions used for parenteral, intradermal, or subcutaneous application can include, for example, the following components: a sterile diluent such as water for injection, saline solution, fixed oils, polyethylene glycols, glycerin, propylene glycol or other synthetic solvents; anti-inflammatory agents; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH value can be adjusted with acids or bases, such as hydrochloric acid or sodium hydroxide.

Compositions as described herein can be administered to the subject one time (e.g., as a single injection or deposition). While the term for administering of at least one compound to prevent pain varies depending on species, and the nature and severity of the condition to be prevented or treated, the compound can be administered to humans for a short term or a long term, e.g. for 1 week to 1 year. For example, administration can be once or twice daily to a subject in need thereof for a period of time, such as one week or one month.

The dosage can vary depending upon known factors such as the pharmacodynamic characteristics of the active ingredient and its mode and route of administration; time of administration of active ingredient; age, sex, health, and weight of the recipient; nature and extent of symptoms; kind of concurrent treatment, frequency of treatment and the effect desired; and rate of excretion.

A therapeutically effective dose can depend upon a number of factors known to those of ordinary skill in the art. The dose(s) can vary, for example, depending upon the identity, size, and condition of the subject or sample being treated, further depending upon the route by which the composition is to be administered, if applicable, and the effect which the practitioner desires. These amounts can be readily determined by the skilled artisan.

In some embodiments, the therapeutically effective amount is at least about 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 150 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight.

A therapeutically effective dose can depend upon a number of factors known to those of ordinary skill in the art. The dose(s) can vary, for example, depending upon the identity, size, and condition of the subject being treated, further depending upon the route by which the composition is to be administered, if applicable, and the effect which the practitioner desires. These amounts can be readily determined by the skilled artisan.

In an embodiment, the recommended daily dose range of a compound as described herein for pain as described herein lies within the range of from about, a daily dose of about 1 mg/body to about 10 g/body, for example about 5 mg/body to about 5 g/body, or for example about 10 mg/body to about 2 g/body of the active ingredient is generally given for treating this disease, and an average single dose of about 0.5 mg to about 1 mg, about 5 mg, about 10 mg, about 50 mg, about 100 mg, about 250 mg, about 500 mg, about 1 g, about 2 g and about 3 g is generally administered. Daily dose for administration in humans for treating or ameliorating pain could be in the range of about 1 mg/kg to about 300 mg/kg.

A compound as described herein, for example a compound of Formula I, or composition comprising the same can be administered to the subject one time (e.g., as a single injection or deposition). Alternatively, administration can be once or twice daily to a subject in need thereof for a period of from about 2 to about 28 days, or from about 7 to about 10 days, or from about 7 to about 15 days. It can also be administered once or twice daily to a subject for a period of 1, 2, 3, 4, 5, 6, 7, 8, 9, 10, 11, 12 times per year, or a combination thereof.

Single unit dosage forms of the disclosure are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal (e.g., cream, lotion, or dermal spray) or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions or solutions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms for parenteral administration to a subject.

The composition, shape, and type of dosage forms of the disclosure will typically vary depending on their use. Further, the dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

For example, a dosage form used in the acute treatment of a disease can contain larger amounts of one or more of the active agents it comprises than a dosage form used in the chronic treatment of the same disease. Similarly, a parenteral dosage form can contain smaller amounts of one or more of the active agents it comprises than an oral dosage form used to treat the same disease. These and other ways in which specific dosage forms encompassed by this disclosure will vary from one another will be readily apparent to those skilled in the art. See, e.g., Remington's Pharmaceutical Sciences, 18th ed., Mack Publishing, Easton Pa. (1990). The methods are non-cytotoxic to the liver.

Any of the therapeutic applications described herein can be applied to any subject in need of such therapy, including, for example, a mammal such as a mouse, a rat, a dog, a cat, a cow, a horse, a rabbit, a monkey, a pig, a sheep, a goat, or a human. In some embodiments, the subject is a mouse, rat, pig, or human. In some embodiments, the subject is a mouse. In some embodiments, the subject is a rat. In some embodiments, the subject is a pig. In some embodiments, the subject is a human.

### Results

### Synthesis Rationale of a Non-Hepatotoxic ApAP Analog, SRP-001

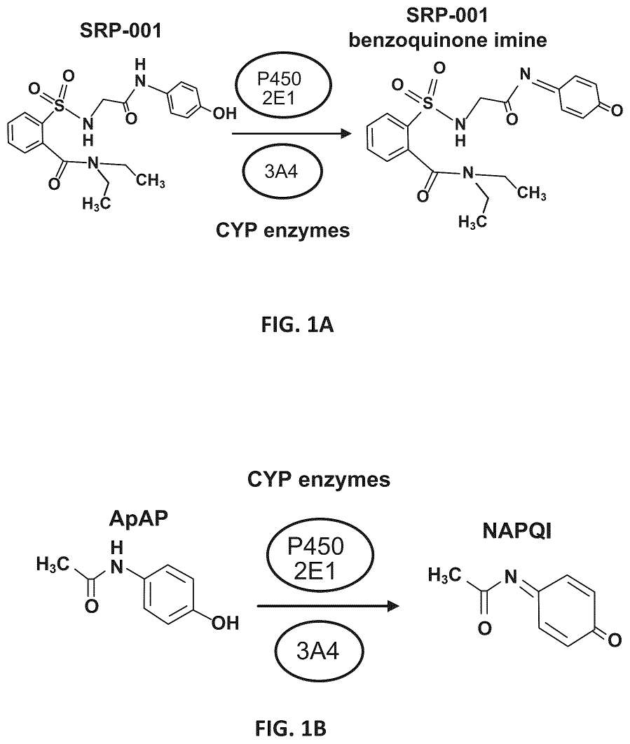

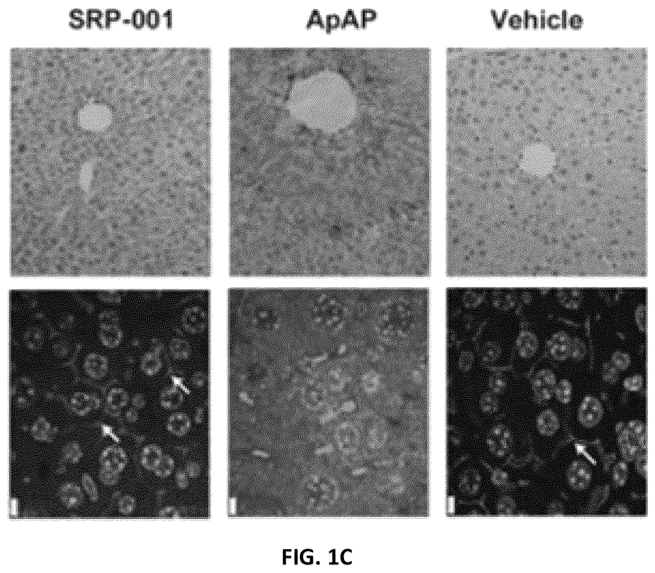

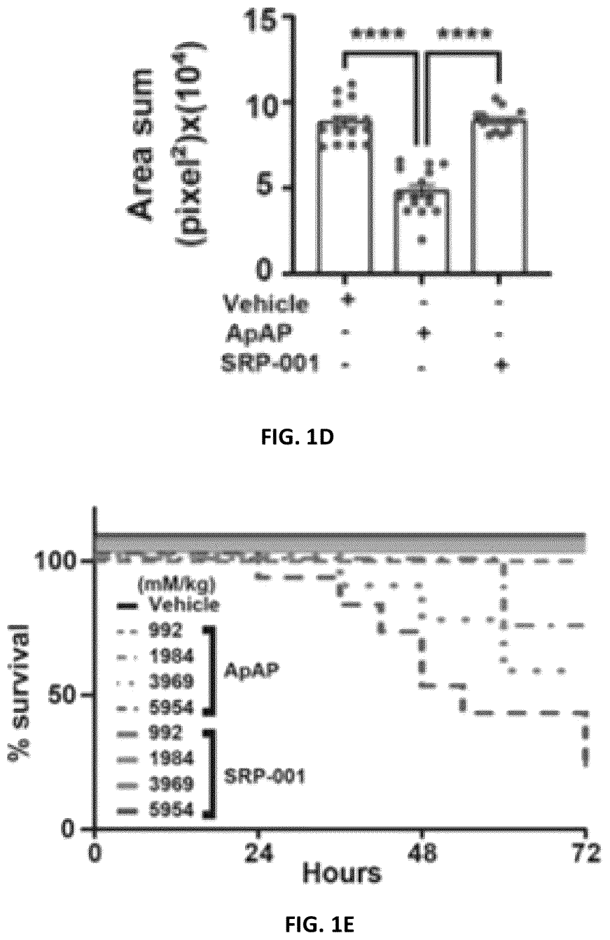

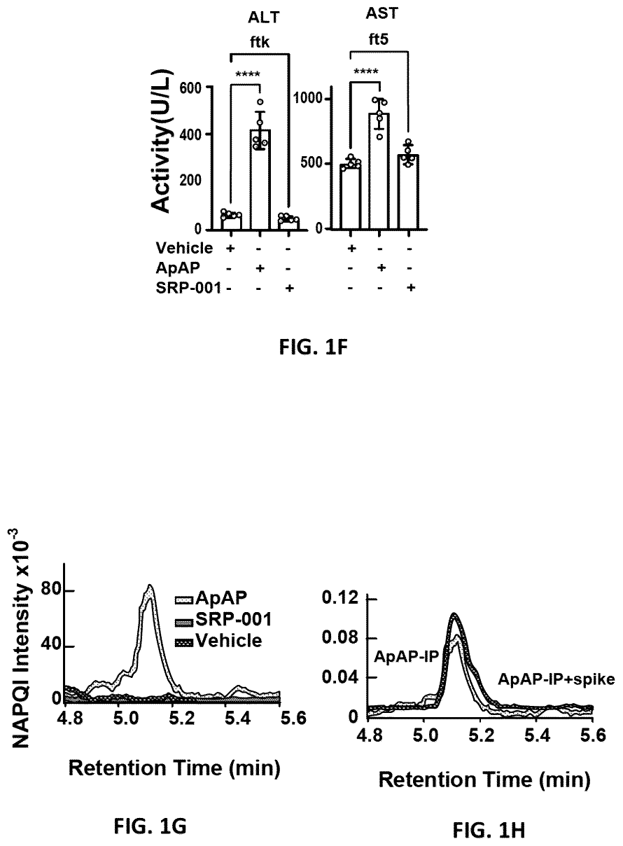

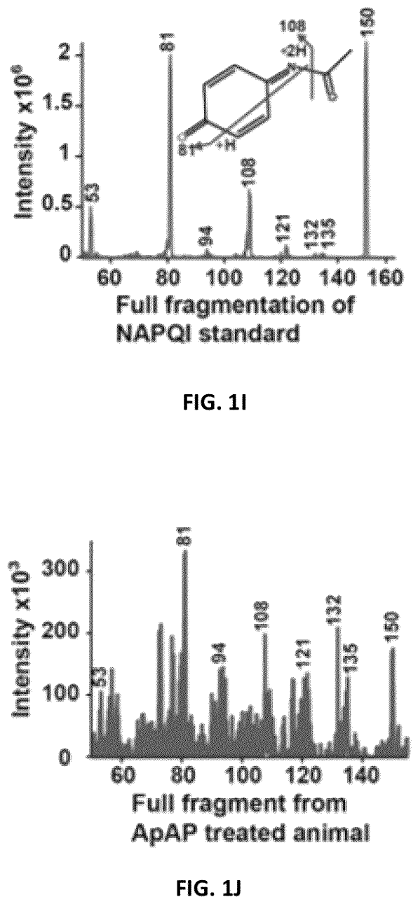

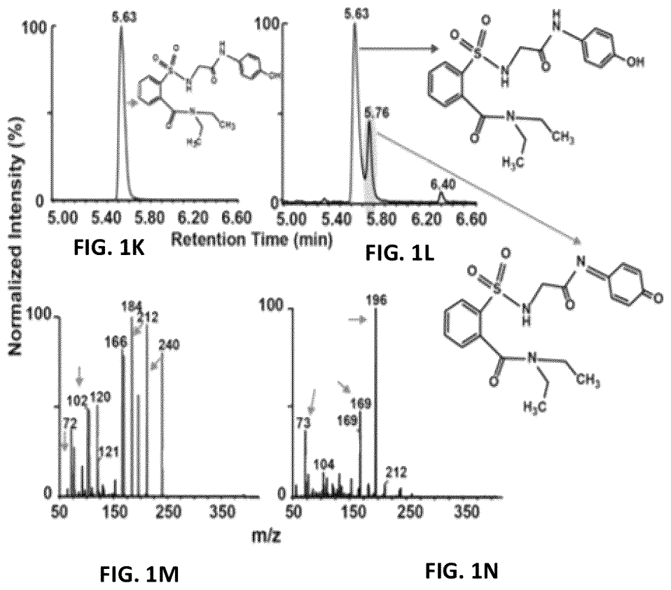

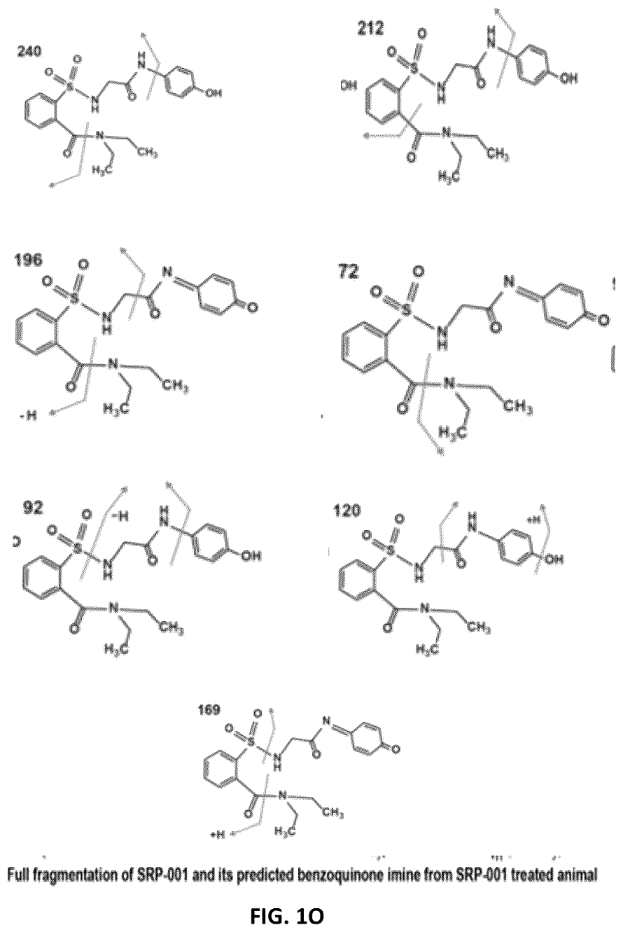

To address ApAP's narrow therapeutic index and the clinical need for safer non-opioid pain relievers, we explored ApAP analogs without hepatotoxicity. Our goal was to circumvent toxicity by developing ApAP analogs that connect a saccharin moiety to ApAP's methyl group. To achieve this, we employed an effective synthesis method that involved opening the ring of the heterocyclic moiety, leading to moderately lipophilic compounds, with the R1 and R2 groups being adjustable to influence lipophilicity. SRP-001, detailed in FIG. 1A and the Experimental section of Online Methods, was selected as the lead clinical candidate due to the absence of in vitro hepatotoxicity and effective in vivo antinociception; the lack of in vitro hepatotoxicity was further validated in vivo.

### SRP-001 is Devoid of Hepatotoxicity Due to Lack of NAPQI Formation and Protection of Hepatic Tight Junctions Integrity

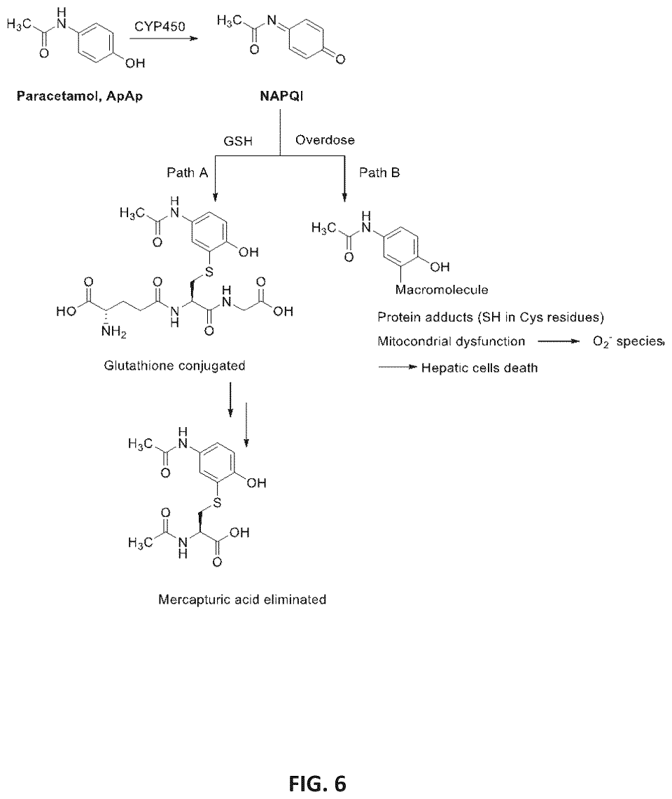

ApAP undergoes oxidation via CYP450 enzymes, specifically CYP 2E1 and 3A4, to generate NAPQI as illustrated in FIG. 1B, FIG. 6. In experiments involving CD-1 mice, hepatotoxicity is evident following exposure to known toxic dosages of ApAP (600 mg/kg). Conversely, mice administered with SRP-001 at an equivalent dosage (600 mg/kg) exhibit no signs of hepatotoxicity.

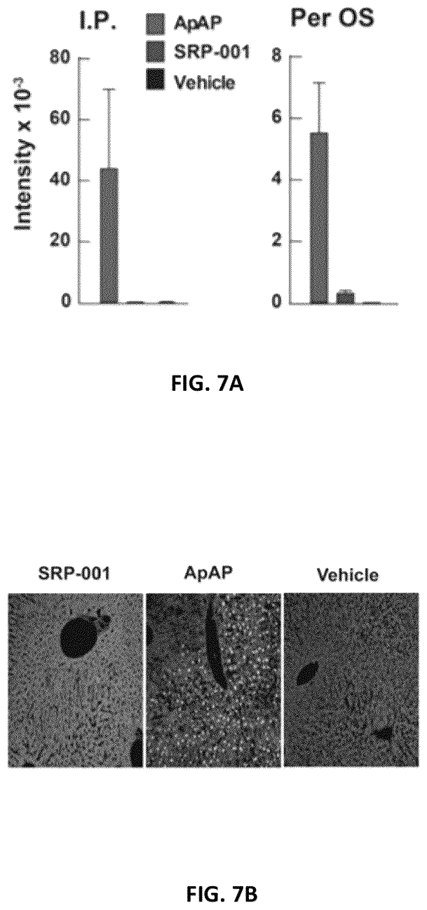

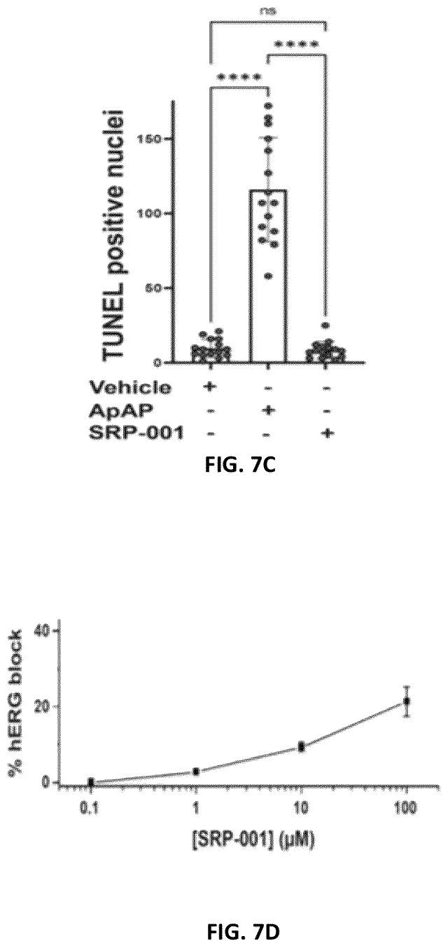

Liver sections of ApAP-treated mice displayed centrilobular necrosis and brown nitrotyrosine/diaminobenzidine-positive staining, while liver sections from mice treated with SRP-001 lack nitrotyrosine staining (FIG. 1C). Moreover, liver sections from SRP-001 treated mice (600 mg/kg) maintained “chicken wire” tight junctions between hepatocytes, as evidenced by Zonula Occludens (ZO-1) staining (FIG. 1C, D), known to be disrupted by ApAP-hepatotoxicity. At a dosage recognized as toxic for ApAP (600 mg/kg), a significant reduction in ZO-1-stained tight junctions is observed, as quantified by the sum of area in pixels\hat{ }2. In contrast, animals administered either the vehicle or SRP-001 (600 mg/kg) retain the integrity of the hepatic ‘chicken wire’ tight junction structure. Concurrently, in vivo assessments reveal elevated levels of liver injury biomarkers alanine aminotransferase (ALT) and aspartate aminotransferase (AST) in mice exposed to toxic dosages of ApAP (600 mg/kg), a phenomenon not observed in SRP-001-treated mice (FIG. 1F). Histological analysis further corroborates these findings, showing a marked presence of TINEL-positive apoptotic nuclei in liver sections from ApAP-treated animals (600 mg/kg), unlike those from SRP-001-treated mice (600 mg/kg) (FIG. 7B, FIG. 7C).

Next, we conducted an equimolar dose-response toxicity analysis to compare the toxicity profiles leading to mortality of ApAP and SRP-001. This analysis was critical for robustly comparing their toxic effects, with a particular focus on mortality at equivalent molar concentrations. Over 72 hours, with assessments every 6 hours, we evaluated the mortality associated with increasing equimolar doses of ApAP and SRP-001 in CD-1 male mice (n=10 per group). The study utilized the established hepatotoxic threshold of ApAP at 600 mg/kg, as identified in our previous work and corroborated by other studies, setting a benchmark for a dose-response curve ranging from 150, 300, 600, to 900 mg/kg of ApAP. Converting these doses to molar concentrations based on the molecular weights of ApAP (151.16) and SRP-001 (405.47), we obtained molar concentrations of 992, 1,984, 3,969, and 5,954 mM/kg for ApAP, corresponding to SRP-001 concentrations of 402, 804, 1,609, and 2,414 mg/kg, respectively. Notably, the placebo and SRP-001 groups showed no mortality across all dosage levels. In contrast, the ApAP groups exhibited a dose-dependent increase in mortality, with 1/10 mice succumbing at 1,984 mM/kg, 3/10 at 3,969 mM/kg, and 7/10 at 5,954 mM/kg by the end of the study period (FIG. 1E). For the dosing group of ApAP (1,984 mM/kg) 1 mouse was found dead around the 60 h time point, for the ApAP (3969 mM/kg) group, 3 mice were dead at time points—36 h, 48 h, and 60 h, and for the ApAP (5954 mM/kg) group, there was a total of 7 mice found dead; out of which 1 mouse was found dead at the respective time points—24 h, 36 h, 42 h, 54 h, and 72 h, and 2 mice found dead at the 48 h time point.

We utilized the Cox proportional hazards regression model to evaluate the impact of increasing equimolar doses of ApAP compared to SRP-001 on mortality in CD-1 male mice. We excluded data from the placebo and lowest dose groups of both ApAP and SRP-001 due to the absence of mortality in these groups. Our primary focus was on the higher dosage levels where differences in mortality were evident. The analysis proceeded in two distinct phases: 1) Group Comparison: Initially, we compared mortality rates between the ApAP and SRP-001 groups across all doses. This comparison, conducted using the likelihood ratio test (LRT), revealed a significant disparity in mortality rates between these groups (χ²(3)=19.2, P=0.0002). Group comparisons via LRT at specific dosages showed significantly higher mortality rates in the ApAP group at 3,969 and 5,954 mM (P=0.0315 and P=0.0003, respectively). In comparison, mortality did not significantly differ between groups at the 1,984 mM dose (p=0.2369). 2) Dose-Response Analysis within ApAP: Subsequently, we examined mortality differences within the ApAP group at 3,969 mM and 5,954 mM dosages versus 1,984 mM. This analysis highlighted a dose-response relationship, indicating that higher doses of ApAP were linked to increased mortality (χ²(2)=8.4, P=0.0151). However, post-hoc tests of individual dosages revealed that mortality is significantly increased only at the 5,954 mM dose compared to 1,984 mM (P=0.0044).

Additionally, we used Kaplan-Meier analysis (FIG. 1E) to plot survival curves for the two groups across various dosages. This approach allowed for a detailed examination of survival probabilities over time, clearly representing how survival rates varied with different dosages. The log-rank test confirmed a statistically significant difference in survival, with the survival curves supporting the observed dose-response increase in mortality with ApAP. Animals treated with equimolar doses of SRP-001 did not exhibit this trend.

Finally, we uncovered that SRP-001's absent hepatotoxicity is due to the lack of formation of the toxic quinonimine metabolite NAPQI (FIGS. 1G-O and FIG. 7A). Serum NAPQI peak is shown at the retention time of 5.1 min on the chromatogram (FIG. 1G). In CD-1 mice injected with a toxic dose (600 mg/kg) of ApAP, a NAPQI peak is seen after ip-injection with ApAP but not in the SRP-001. The control, ip-ApAP sample with a standard spiked NAPQI, demonstrates the same retention time in the chromatogram (FIG. 1H). LC-MS/MS full fragmentation spectrum from ip-ApAP sample's NAPQI peak is depicted in (FIG. 1I). This was further confirmed with full fragmentation pattern of NAPQI standard. The spectrum matches the standard as the retention time peaks at 5.1 min on the chromatogram (FIG. 1J-L). We also identified the predicted SRP-001 benzoquinoimine from SRP-001-treated animals and full fragmentation pattern of SRP-001 in positive ionization mode in LC-MS/MS. The retention peaks at 5.76 min on the chromatogram indicates the predicted benzoquinoimine of SRP-001 (FIG. 1K-FIG. 1O).

### In Vivo Antinociception and Antipyresis are Comparable for ApAP and SRP-001

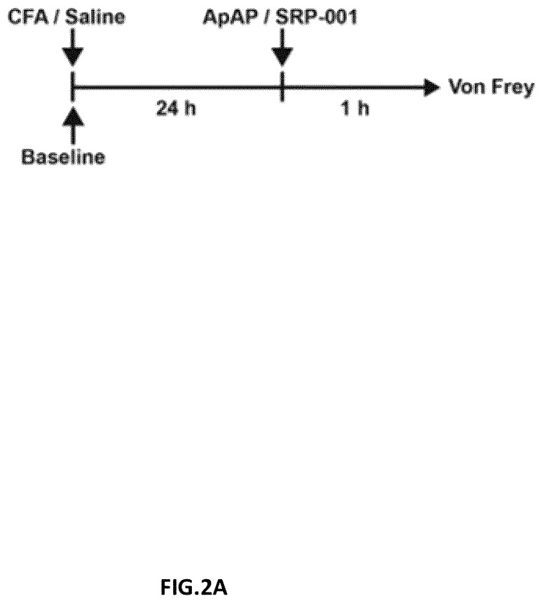

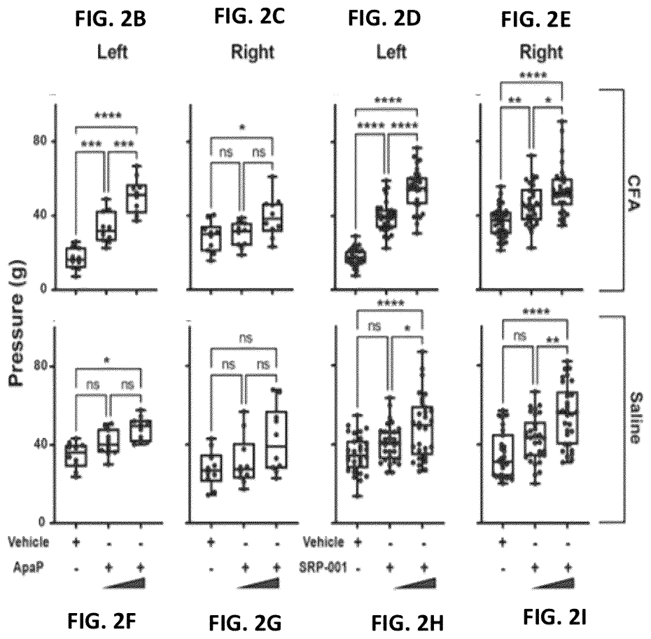

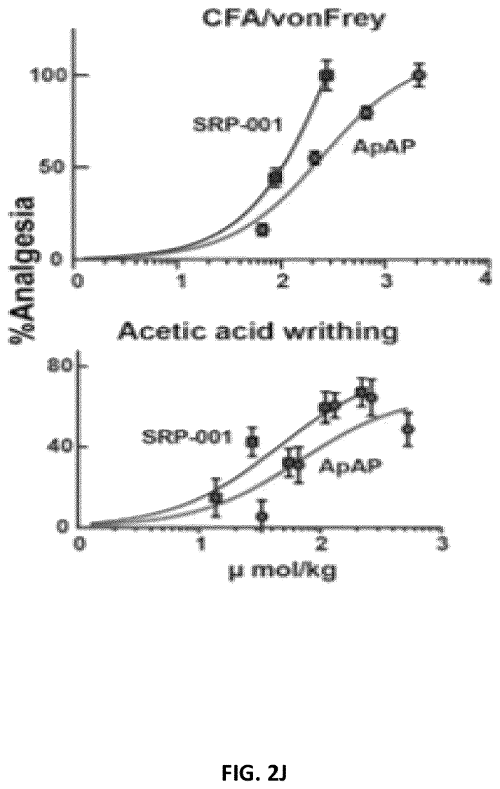

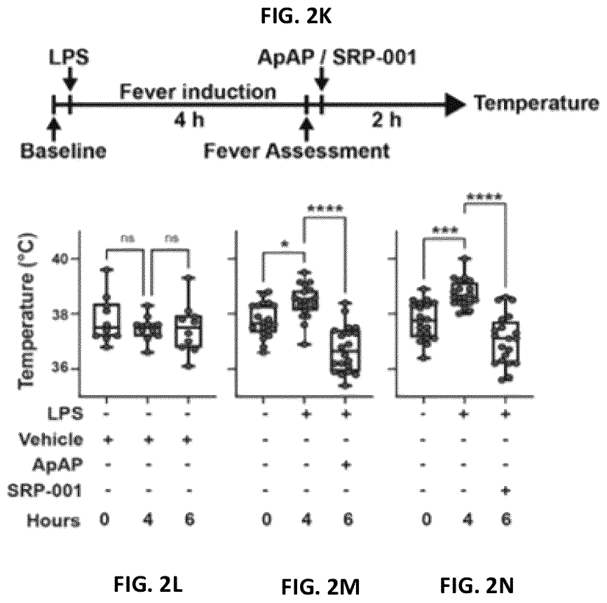

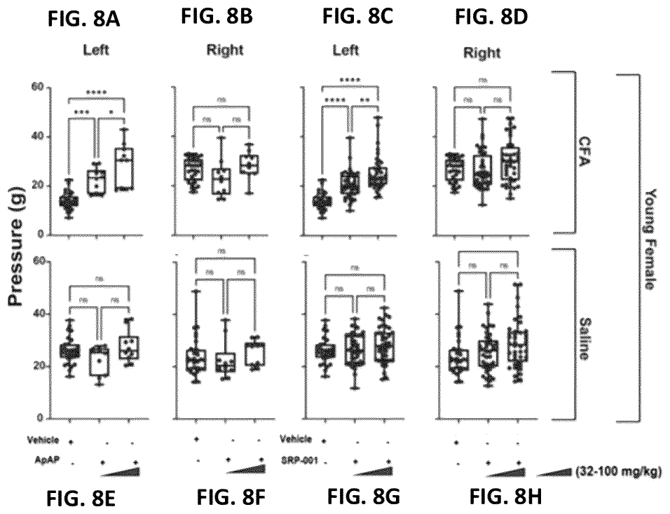

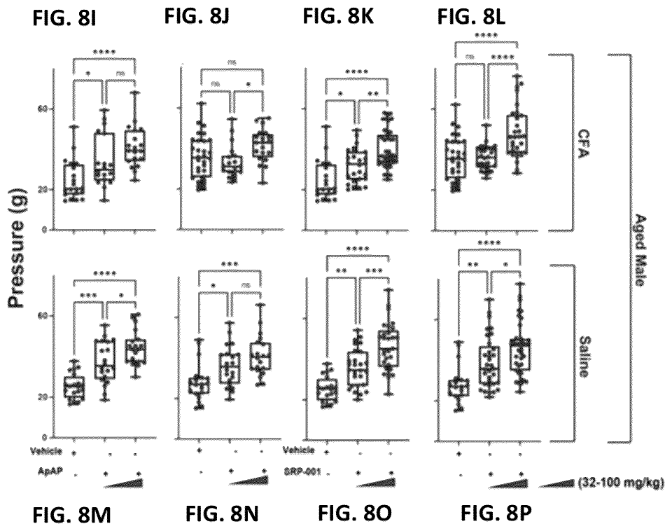

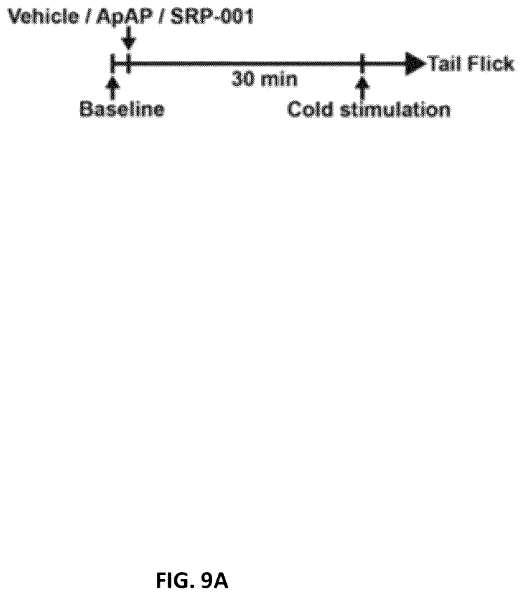

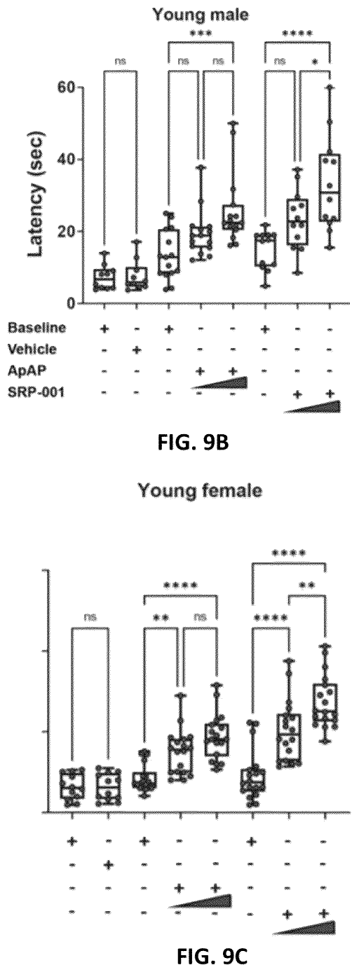

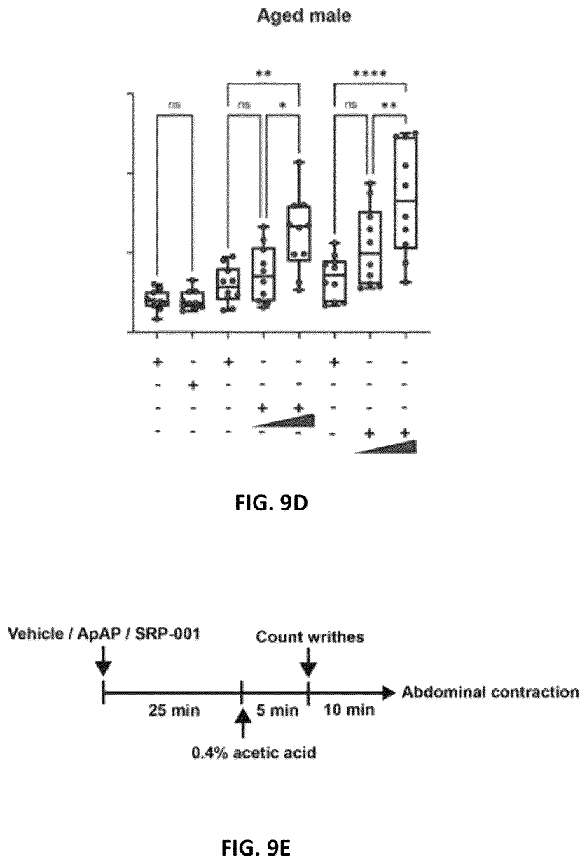

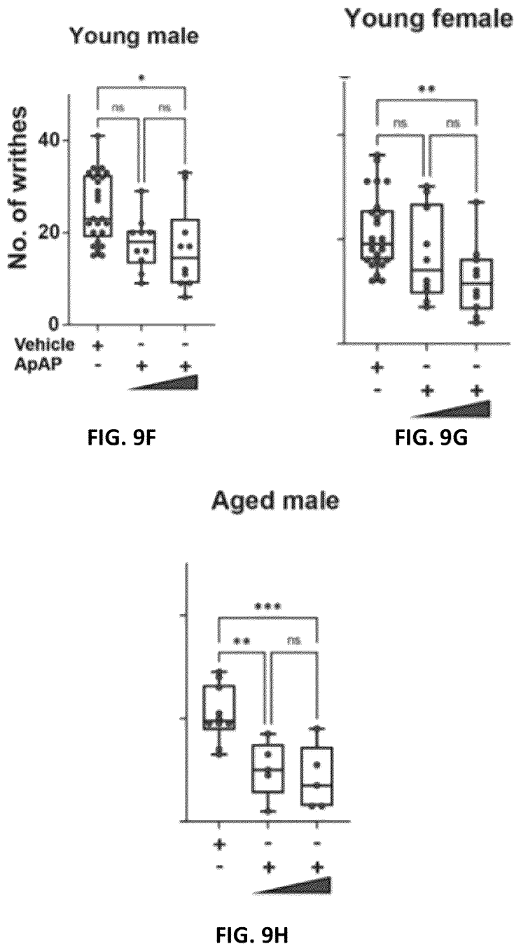

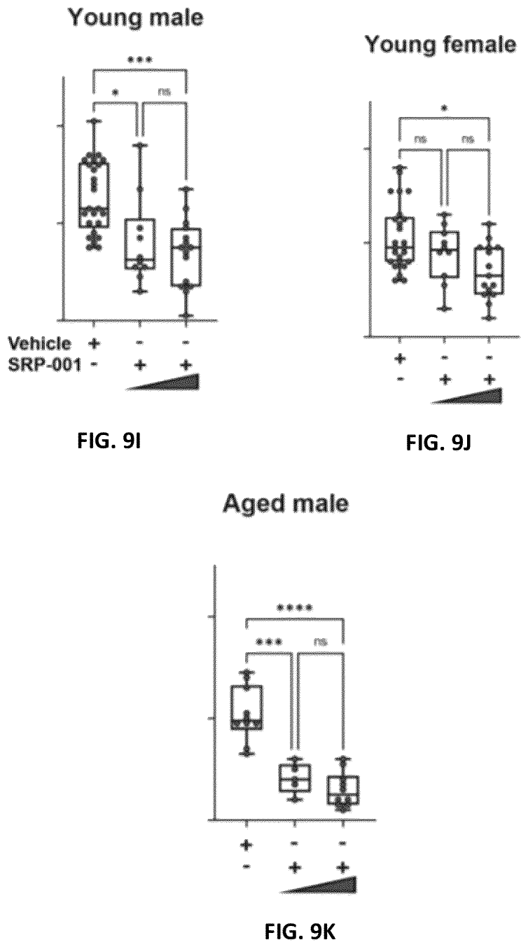

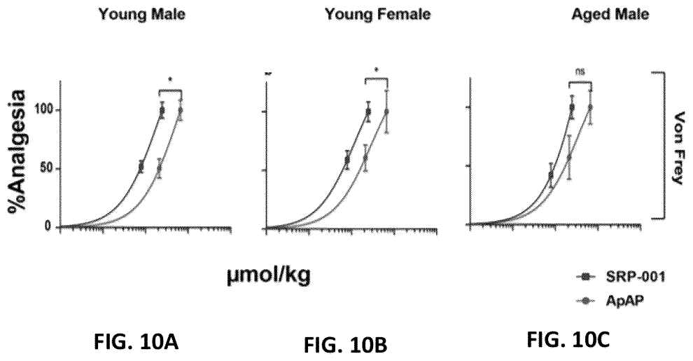

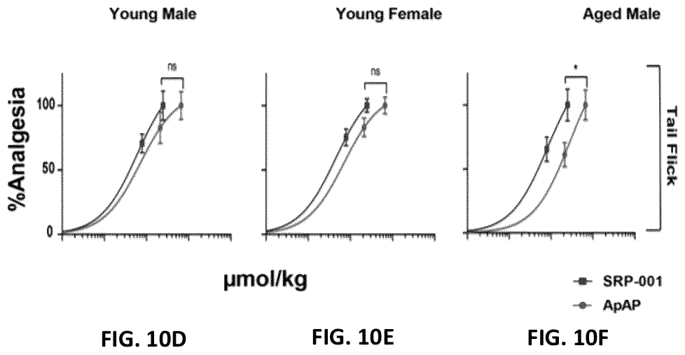

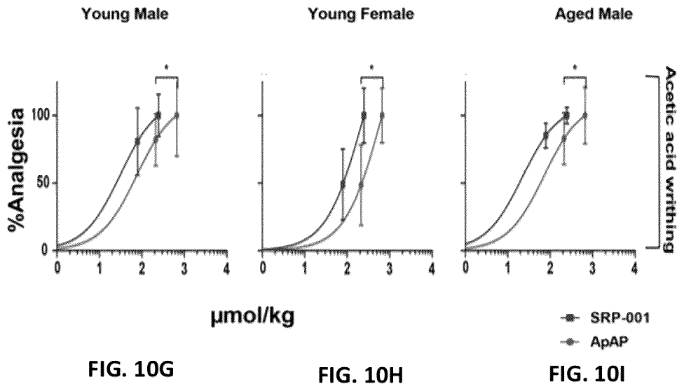

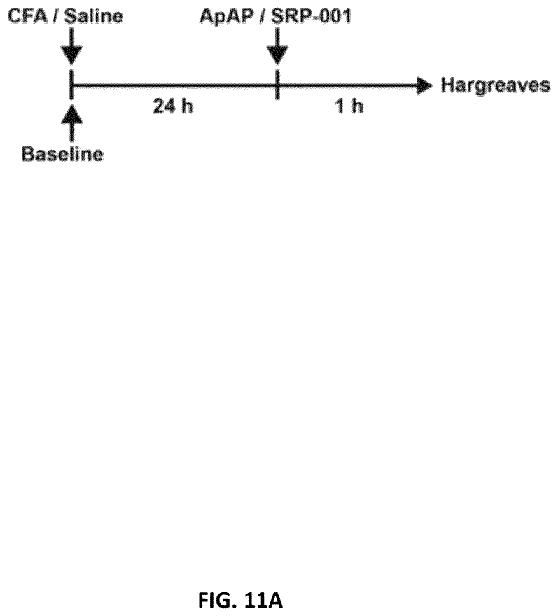

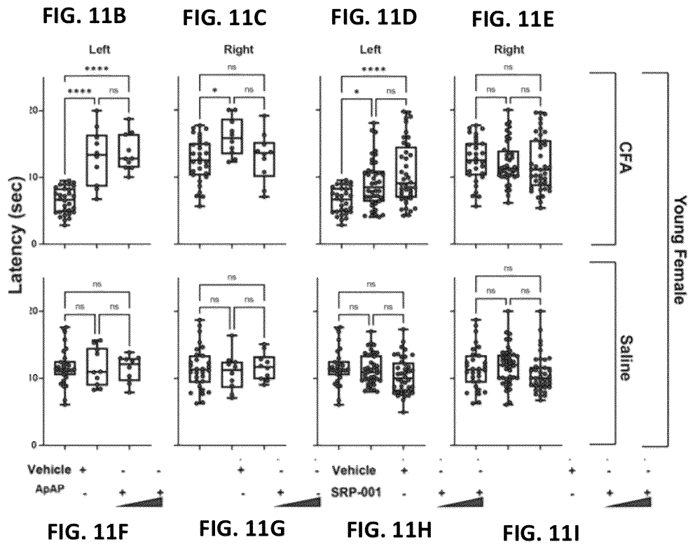

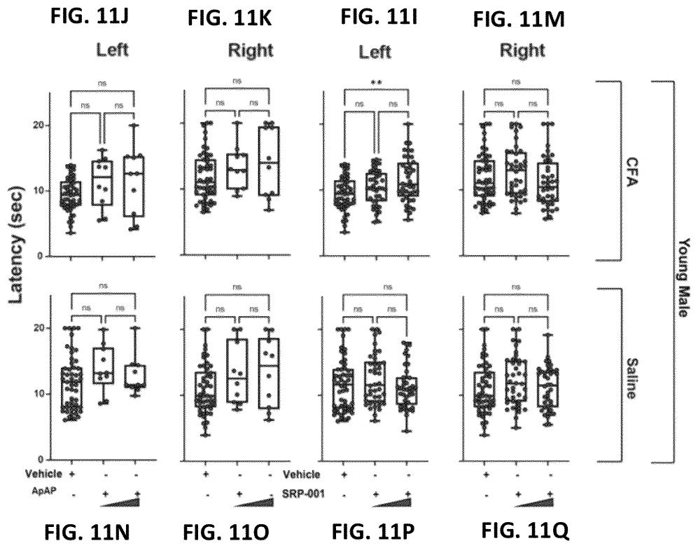

We measured in vivo antinociception in mice (CD1 and C57BL/6) and rats (Sprague Dawley) using complementary in vivo animal models of pain and nociceptive sensitivity. These included the complete Freund's adjuvant (CFA) induced inflammatory pain/von Frey model (FIG. 2, FIG. 8), tail flick somatic, and abdominal writhing visceral assays (FIG. 9), along with electronic von Frey (eVF) and Hargreaves determination of mechanical and thermal sensitivity (FIG. 11, 12). the cohort of young male rats, (FIG. 2B-2I) the average baseline hind paw withdrawal threshold to the eVF filament was 35 g pressure. After CFA injection, the withdrawal threshold was 18 g for the injected left hind paw (indicating mechanical hyperalgesia), while for the un-injected right hind paw, the withdrawal threshold remained at 35 g pressure (FIG. 2B, 2C, 2F, 2G) for rats treated with ApAP, and (FIG. 2D For FIGS. 2E, 2H and 2I) for the rats treated with SRP-001. SRP-001 and ApAP showed comparable antinociceptive activity for this cohort of young female rats (FIG. 8A, 8C). In SRP-001-treated animals, for the CFA-injected paw, the threshold for paw withdrawal increased from 18 g to 40 g and subsequently to 55 g (32 mg/kg and 100 mg/kg, respectively) for dose. In ApAP-treated animals, for the CFA-injected paw, the threshold for paw withdrawal increased from 18 g to 34 g and subsequently to 50 g (32 mg/kg and 100 mg/kg) for dose, respectively. SRP-001 and ApAP showed comparable antinociceptive activity in a cohort of young and aged animals (FIG. 8A-8P). Equimolar (μmol/kg) ED₅₀ curves were generated using non-linear regression analysis showing that SRP-001 shifted curves to the left as compared to ApAP (FIG. 2J and FIG. 10).

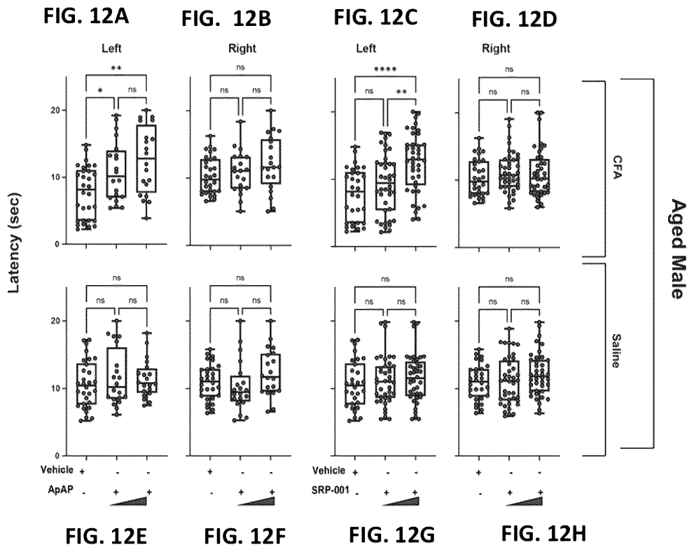

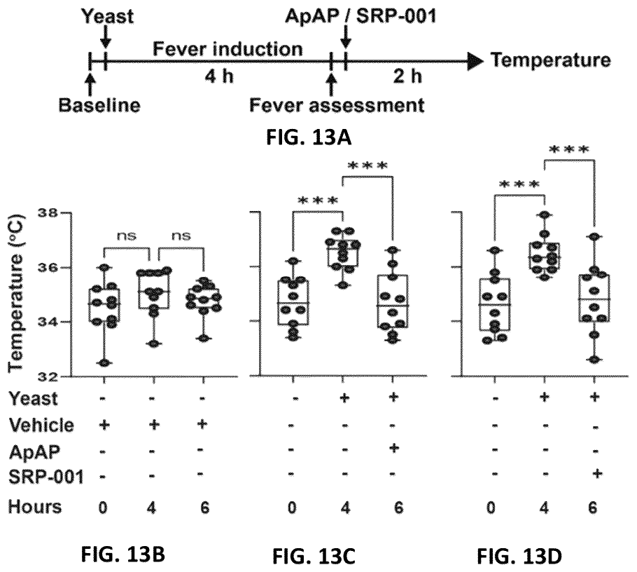

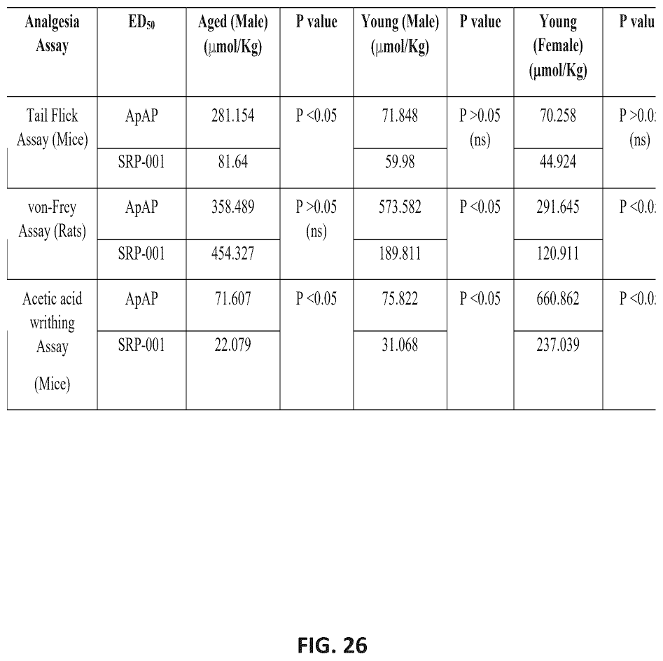

Next, we assessed SRP-001's antinociception in two other pain models, the tail flick and acetic acid writhing assays in mice. We observed antinociceptive effects using the tail-flick somatic pain assay (FIG. 9A-9D), noting an increased tail withdrawal time (latency) in SRP-001 or ApAP-treated (32 and 100 mg/kg) CD1 (young male and female) or C57BL/6 (aged male) mice to cold stimulation of the tail tip. For the cold stimulation test, half of the tail was immersed in ice-cold water maintained at 4° C. The acetic acid-induced abdominal writhing assay, a model of visceral pain in CD1 and C57BL/6 mice in which contraction of the abdominal muscles and stretching of the hind limbs is induced in response to ip injection of 0.4% acetic acid (FIG. 9e-k). SRP-001 or ApAP (32 and 100 mg/kg)-treated CD1 (young male and female) or C57BL/6 (aged male) mice, but not vehicle, also experienced antinociception. Notably, all three antinociception models demonstrate comparable or improved ED₅₀ for SRP-001 compared to ApAP at equimolar (μmol/kg) amounts (FIG. 10). Antinociception was also confirmed in the Hargreaves assay in young female and male rats (FIG. 11 and FIG. 26 Table 1) and aged male rats (FIG. 12). Lastly, SRP-001 induces similar antipyresis to ApAP in the LPS fever induction model (FIGS. 2K-2N) and the baker's yeast fever induction model in CD1 mice (FIG. 13).

### Antinociception is Mediated Via N-Arachidonoylaminophenol (AM404) Production in the CNS Periaqueductal Gray Area

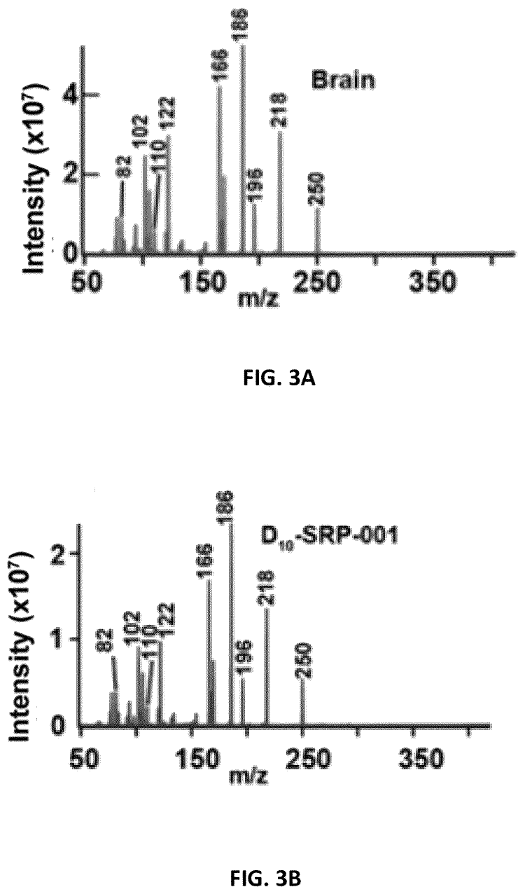

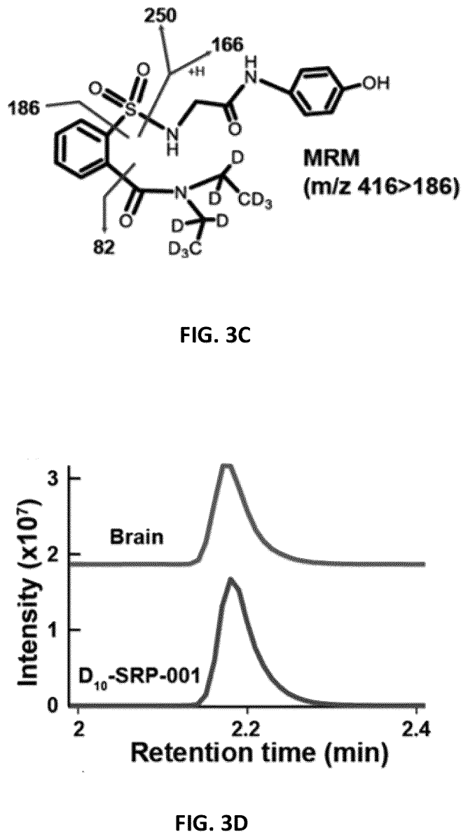

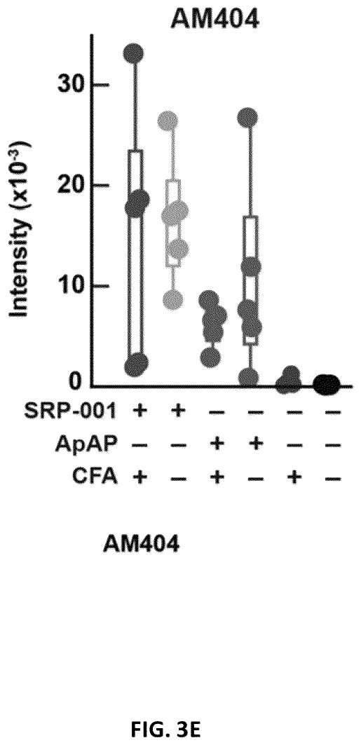

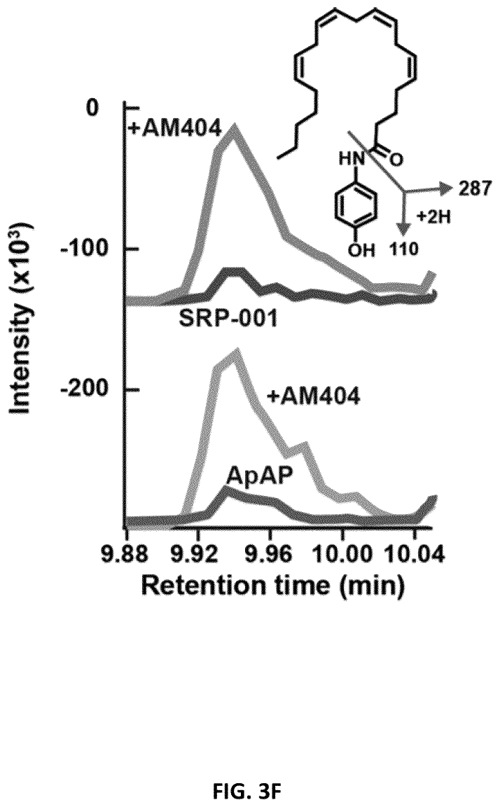

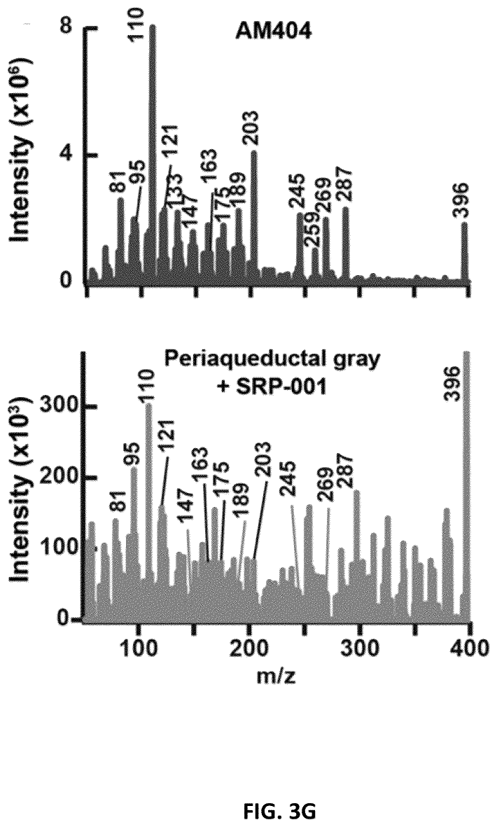

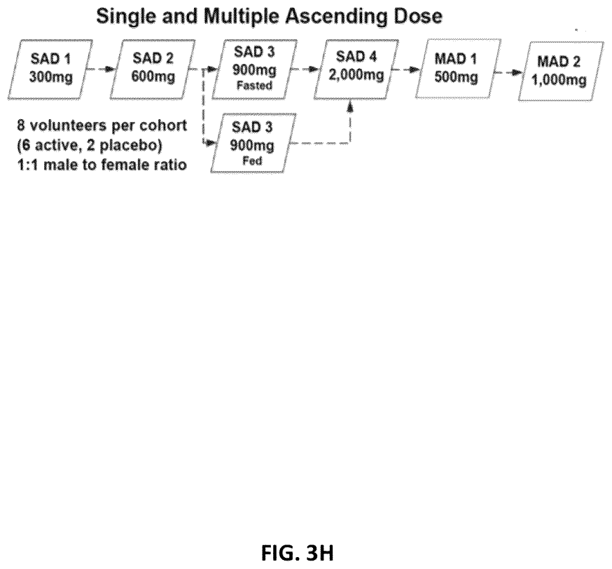

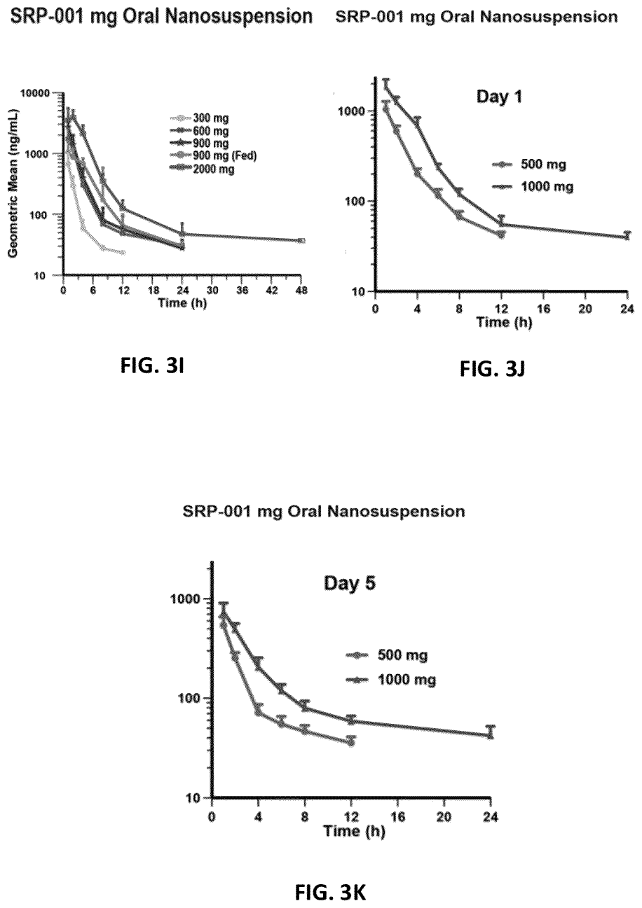

Next, we sought to define the uptake of SRP-001 in the nociception area of the brain, the midbrain's periaqueductal (PAG) area. Thirty min following ip-SRP-001 administration (32 mg/kg), the PAG was harvested and analyzed by LC/MS-MS. D₁₀-SRP-001 was used to match the fragmentation pattern while monitoring analytes. The full fragmentation spectrum of D₁₀-SRP-001 detected in the brain matches the standard SRP-001 spectrum (FIG. 3A-3B). Further, the structure of D₁₀-SRP-001 (FIG. 3C) shows fragmentation patterns of D₁₀-SRP-001 that match the spectra shown in the brain and with the deuterated standard (FIG. 3D).

ApAP induces antinociception in the midbrain's region via the formation of its metabolite, N-arachidonoylaminophenol (AM404). AM404 acts on transient receptor potential vanilloid-1 (TRPV1) and cannabinoid 1 receptors in the brain to produce antinociceptive effects. Thus, we quantified PAG AM404 production by LC-MS/MS, post dosing with ApAP or SRP-001. Notably, the highest AM404 production in the PAG is in animals treated with SRP-001 compared to ApAP (FIG. 3E). Peak levels of AM404 are observed in animals co-treated with CFA and SRP-001, followed by those receiving a combination of vehicle (0.9% saline) and SRP-001. Notably, AM404 production is diminished in the periaqueductal gray region of rats administered CFA and ApAP. Minimal to undetectable levels of AM404 are found in the periaqueductal gray region of rats treated solely with either CFA or vehicle. The AM404 structure and its major fragments (FIG. 3f), wherein AM404 peaks are confirmed by co-spiking AM404 standard to the SRP-001 and ApAP injected samples. The top panel represents the AM404 standard, sourced from a pure analytical standard of AM404 (10 ng/mL, Cayman Chemical, Ann Arbor, MI). The bottom panel depicts AM404 levels following intraperitoneal (IP) injection of SRP-001 at a concentration of 32 mg/kg, with animals sacrificed 30 minutes post-injection, consistent with the CFA/von Frey analgesia model. Subsequent to whole-brain extraction, the periaqueductal gray region was specifically isolated for analysis. The major peaks detected by LC-MS/MS demonstrate that the peak 9.9 min on the chromatogram is the AM404 compound; these peaks align with the AM404 fragments (FIG. 3G).

### Phase 1 Trial Results

A randomized, double-blind, placebo-controlled Phase 1 trial (ClinicalTrials.gov identifier: NCT05484414) was conducted to assess the safety, tolerability, food effect, pharmacokinetics (PK), and pharmacodynamics (PD) of single and multiple ascending doses of SRP-001 (Quotient Sciences, Miami FL USA; FIG. 3H and Online Methods). The trial was conducted in accordance with the International Council for Harmonisation of Technical Requirements for Pharmaceuticals for Human Use (ICH), Guideline for GCP: Consolidated Guidance (E6), attained Institutional Review Board approval, and all applicable regulatory requirements for Phase 1, First in Human clinical trials. Safety measurements and plasma samples to determine PK parameters were collected throughout the study for all subjects and reviewed by the Safety Review Committee. PK stopping criteria were established to assure that systemic exposure to SRP-001 was less than or equal to the systemic exposure achieved at the NOAEL doses in the 28-day repeat-dose toxicology studies in the rat and dog. The trial's Safety Advisory Committee has approved the publication of the Phase 1 results.

SRP-001 was administered as an oral nanosuspension to 56 healthy volunteers across five Single Ascending Dose (SAD) and two Multiple Ascending Dose (MAD) cohorts, with 42 subjects receiving SRP-001 and 14 receiving a placebo. SRP-001 was well tolerated, with no serious adverse events (SAEs) reported, as detailed in Table 1. Comprehensive safety assessments, including vital signs, physical examinations, continuous electrocardiogram monitoring, and a wide range of laboratory tests (encompassing liver and kidney function, clinical chemistries, coagulation profiles, hematology, and urinalysis), revealed no abnormalities, indicating a favorable safety profile.

Pharmacokinetic (PK) analysis showed a dose-proportional or super-proportional increase in the geometric mean maximum concentration (Cₘₐₓ) and area under the curve (AUC_{(0-∞)} values across dosing ranges from 300 mg to 2,000 mg, followed by a mono- or bi-phasic decline in concentrations (FIG. 3I-3K). As SRP-001 is increased from 300 to 2,000 mg, the maximum and overall plasma exposure of SRP-001 based on geometric mean (geometric CV %) Cₘₐₓ, AUC_{(0-∞)}, and geometric mean half-life (T_{1/2}) were: 665 ng/mL, 1630 ng·h/mL and 2.4 h (300 mg fasted); 1740 ng/mL, 5840 ng·h/mL and 5.0 hours (600 mg fasted); 2700 ng/mL, 6970 ng·h/mL, and 3.3 hours (900 mg fasted); 1200 ng/mL, 5910 ng·h/mL, and 3.8 hours (900 mg fed); and 4590 ng/mL, 19600 ng·h/mL, and 6.0 hours (2000 mg fasted), respectively.

In the MAD cohorts, the geometric mean T_{1/2} was 3.06 hours on Day 1 and 4.92 hours on Day 5 for the 500 mg dose, and 4.05 hours and 9.77 hours, respectively, for the 1,000 mg dose on Day 1 and Day 5.

### Cell Heterogeneity in Midbrain PAG Area

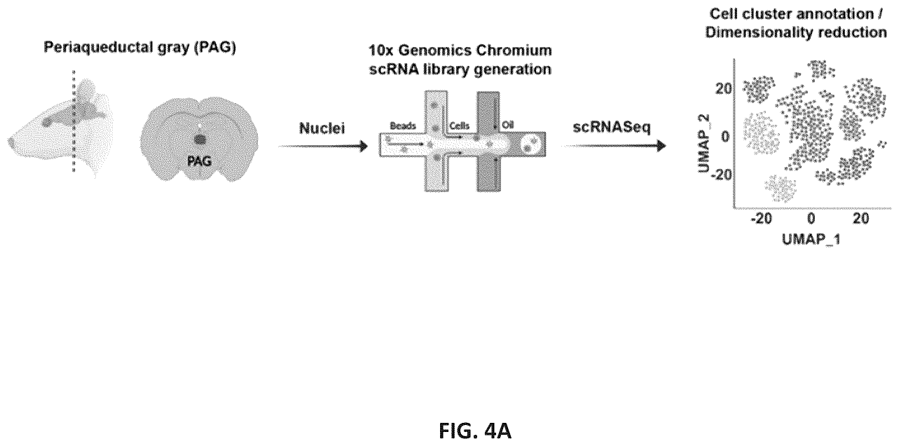

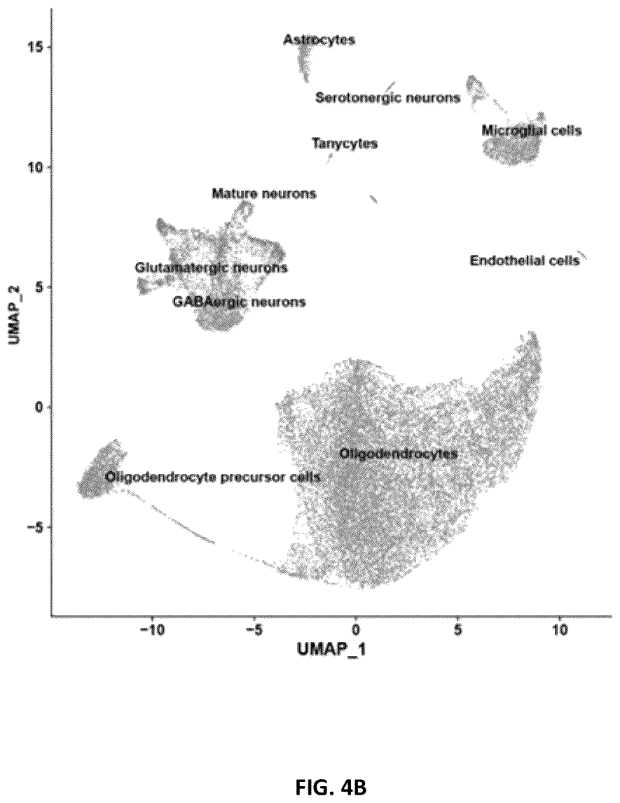

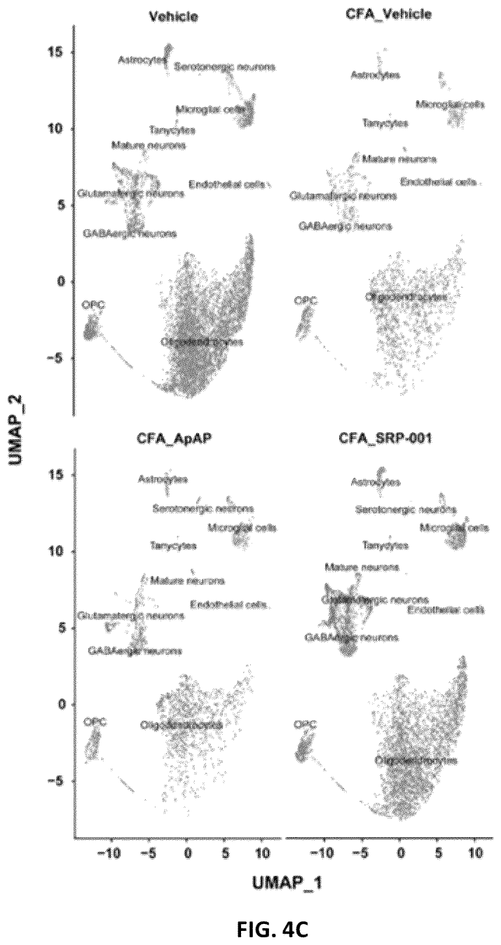

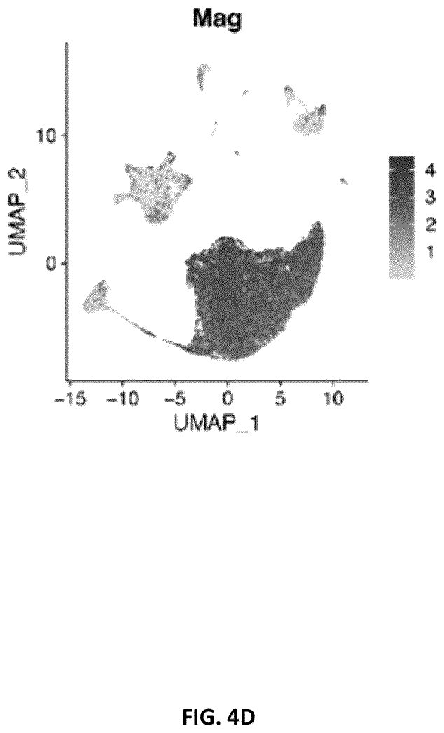

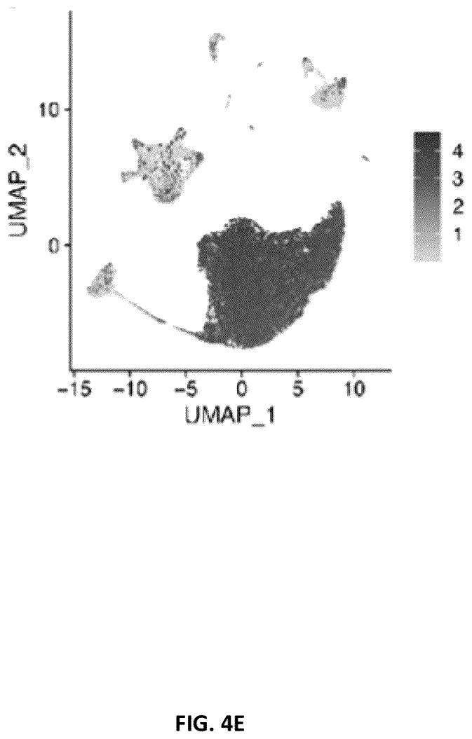

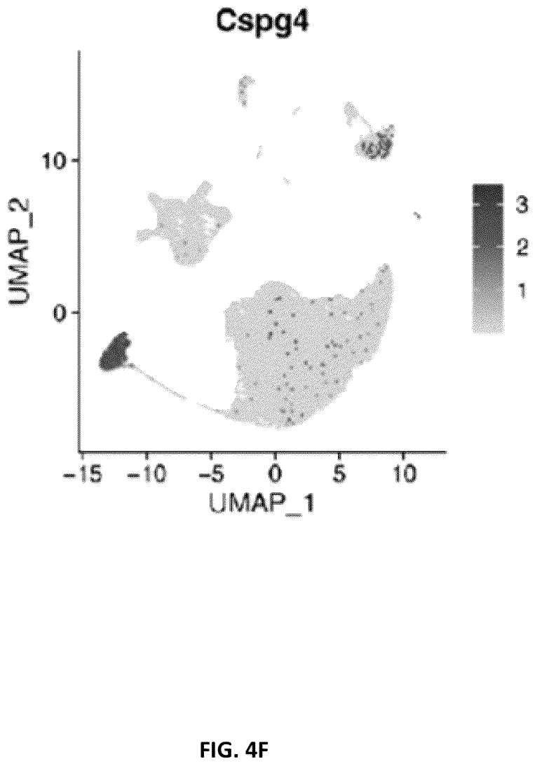

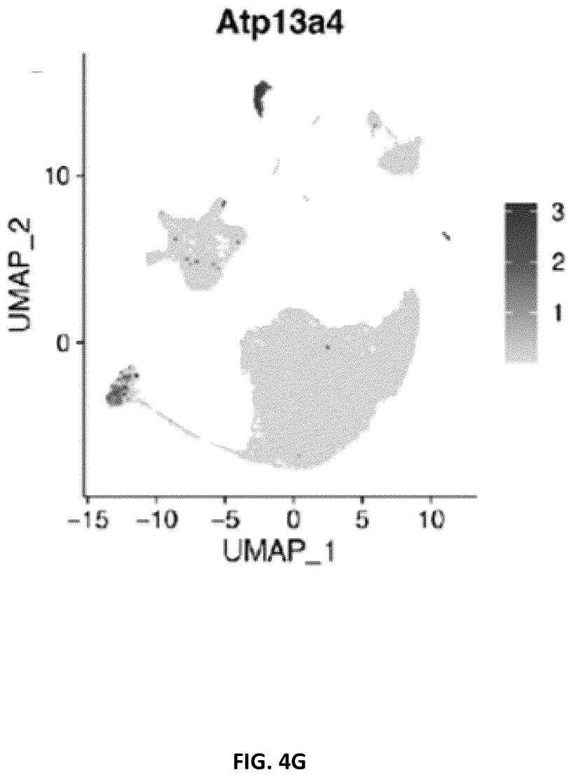

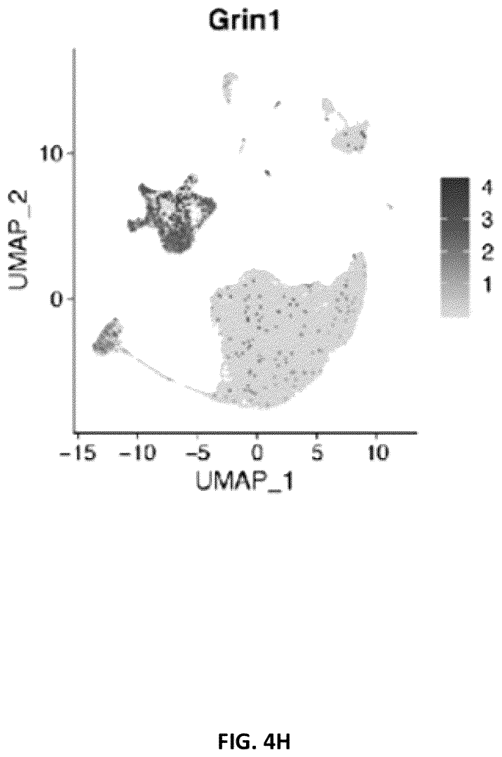

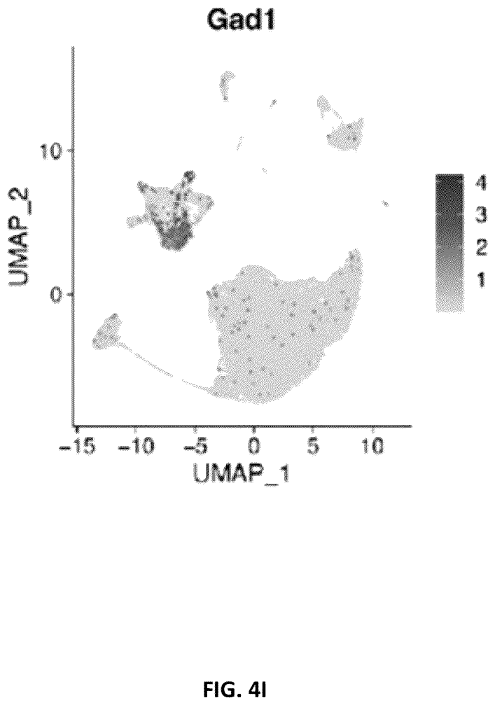

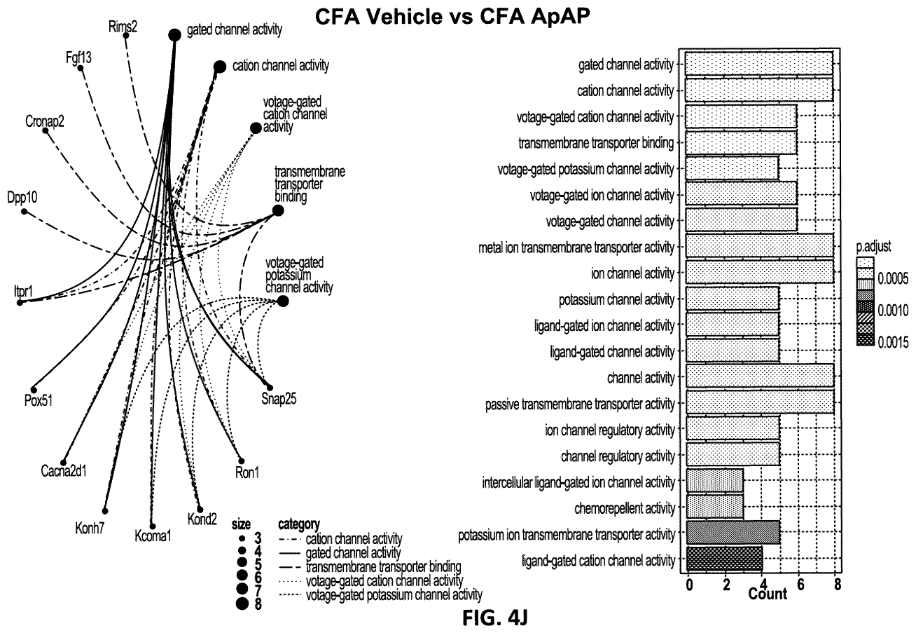

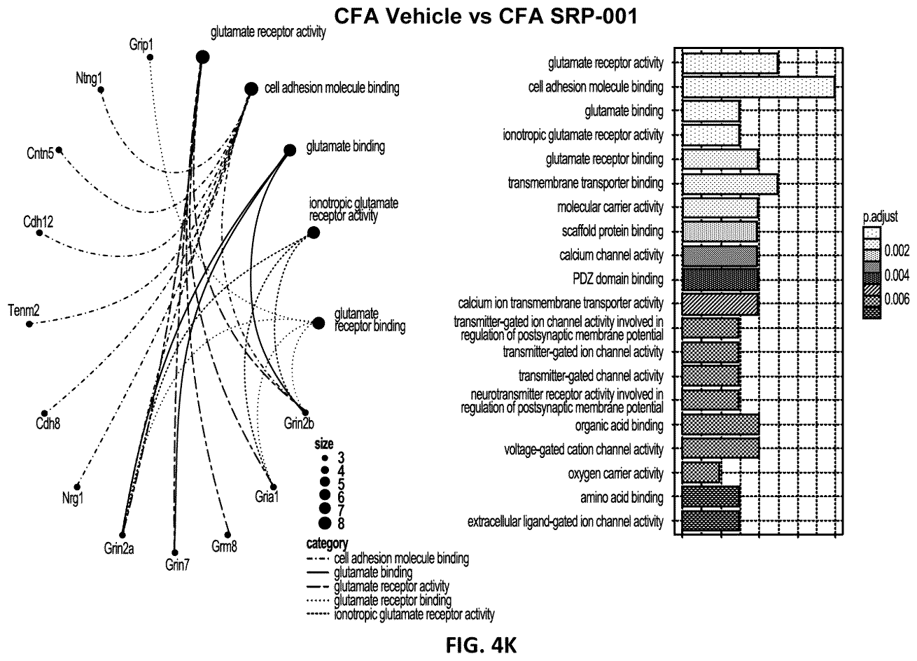

Subsequently, we endeavored to explore the single-cell transcriptome of the eVF rat mid-brain PAG region to further define the mechanisms of action (MOA) of chronic inflammatory pain and, ultimately, SRP-001- and ApAP-mediated pain modulation. Left hind paws of Sprague Dawley rats were injected with 150 μl of 50% complete Freund's adjuvant (CFA), freshly diluted with sterile saline on the day of administration, to induce conspicuous long-standing inflammatory pain in the rats' hind paws, along with mechanical hyperalgesia tested using eVF. to induce local inflammation and a chronic pain state. We dosed the rats with ApAP or SRP-001 (100 mg/kg), and after Ih, they were euthanized to dissect the PAG from flash-frozen brains for 10×Genomics single-nuclei RNA library preparation (FIG. 4A).

### Data Pre-Processing Results

Uniform Manifold Approximation and Projection (UMAP) was used to perform dimensionality reduction and the embedded cells were arranged according to expression profile similarity in a 2-D UMAP scatter plot to show their distribution. The UMAP plot shows that for each sample—Vehicle, CFA_Vehicle, CFA_ApAP, and CFA_SRP-001—similar cell clusters were retained, and annotated into 10 principal cell types—astrocytes, serotonergic neurons, glutamatergic neurons, GABAergic neurons, mature neurons, tanycytes, endothelial cells, microglia, oligodendrocytes, and oligodendrocyte precursor cells (OPCs) (FIG. 4B-4C). Cells were annotated with differentially expressed marker genes using R package scType described in Methods (FIG. 4D-4I). Feature plots for the top marker gene in each major cluster were made, and UMAP plots for annotated clusters for each group show differences in cell type distribution and quantity (FIG. 4D-4I).

### Gene Ontology Enrichment Analysis

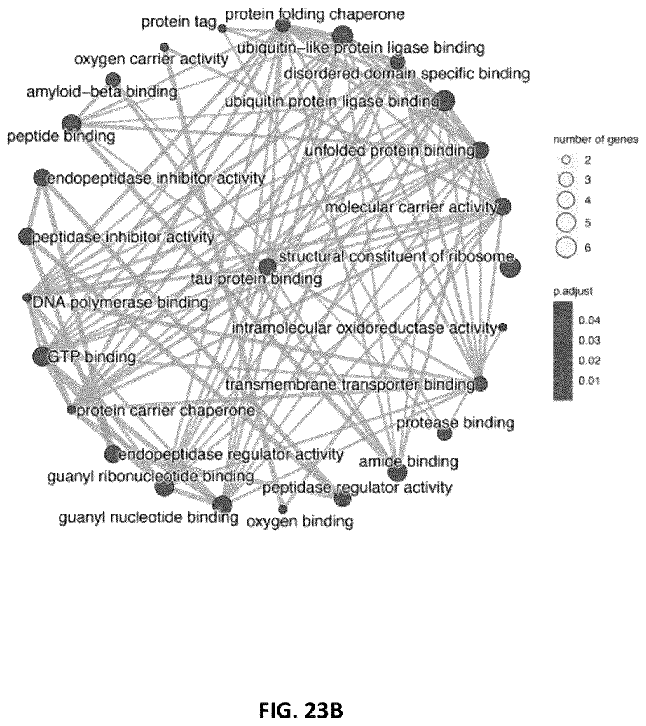

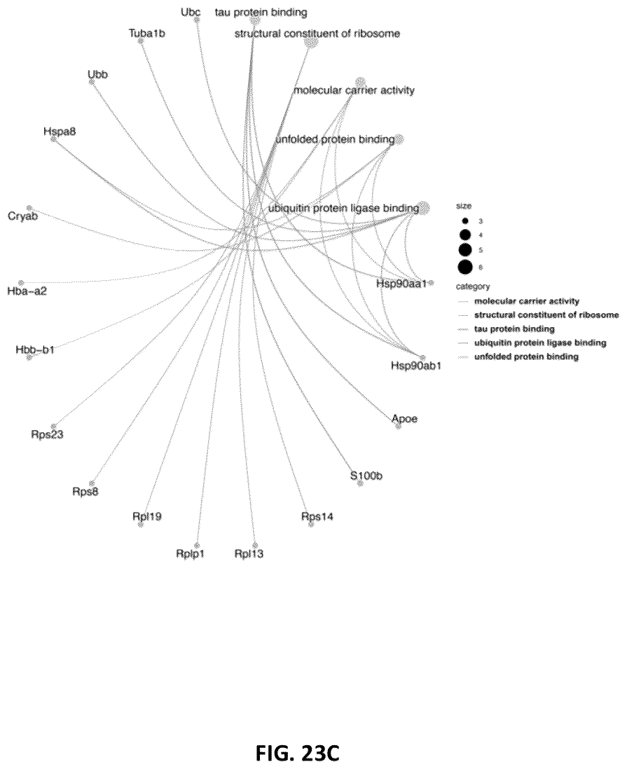

Gene ontology (GO) utilizes the GO knowledgebase to interpret large-scale molecular biology experiments with the endpoint of defining statistically significant similarities or differences between experimental conditions. The knowledgebase provides computational representation of the function of genes and how the genes contribute to a biologic process. Differential expression of genes between Vehicle and CFA_Vehicle treated groups showed enrichment in tau protein binding, molecular carrier activity, peptide binding, GTP binding, and amyloid-beta binding as well as other GO terms (FIG. 23A). The linkage between these terms based on similar genes are shown, and the specific gene role, in particular, GO terms was visualized (FIG. 23B-23C). Differential expression of genes involved in multiple types of gated channel, cation/ion channel, and potassium channel activity was observed from the comparison of CFA_Vehicle and CFA_ApAP (FIG. 4J). The comparison between CFA_Vehicle and CFA_SRP-001 defined differentially expressed genes enriched in GO terms for glutamate receptor activity, glutamate binding, calcium channel activity, and multiple other terms related to ion channel activity like the observed terms from CFA_ApAP (FIG. 4K).

### Principal Component Analysis

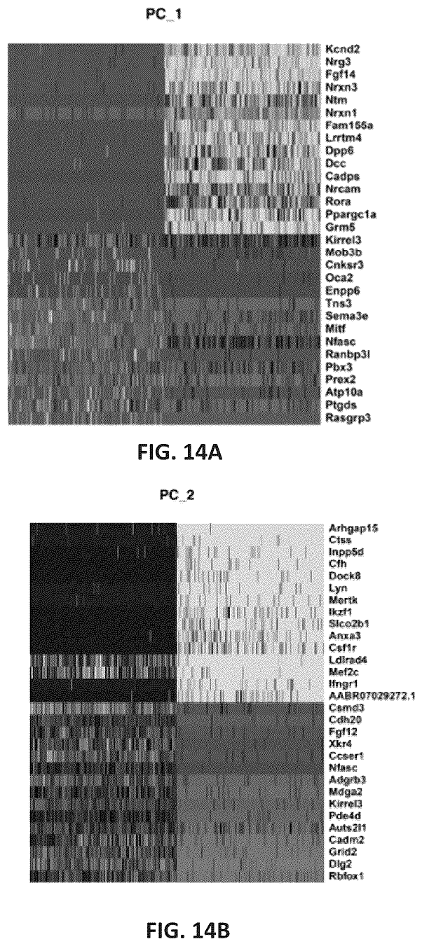

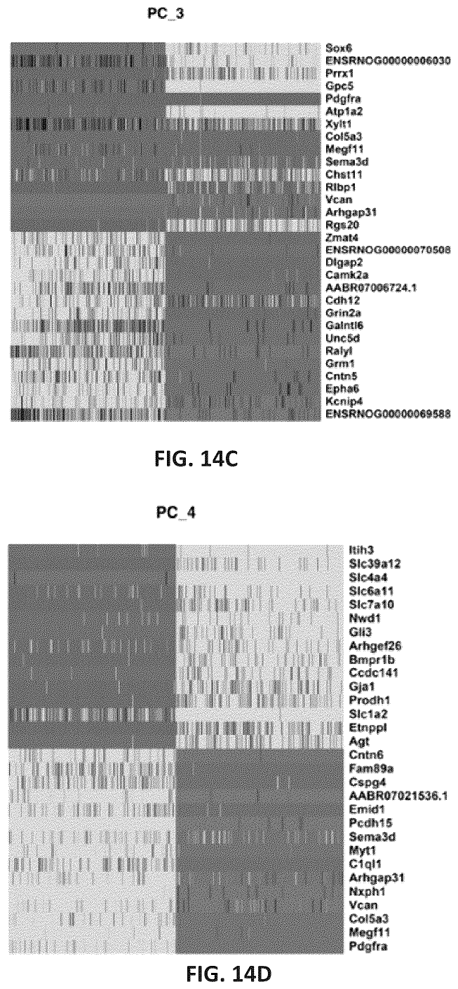

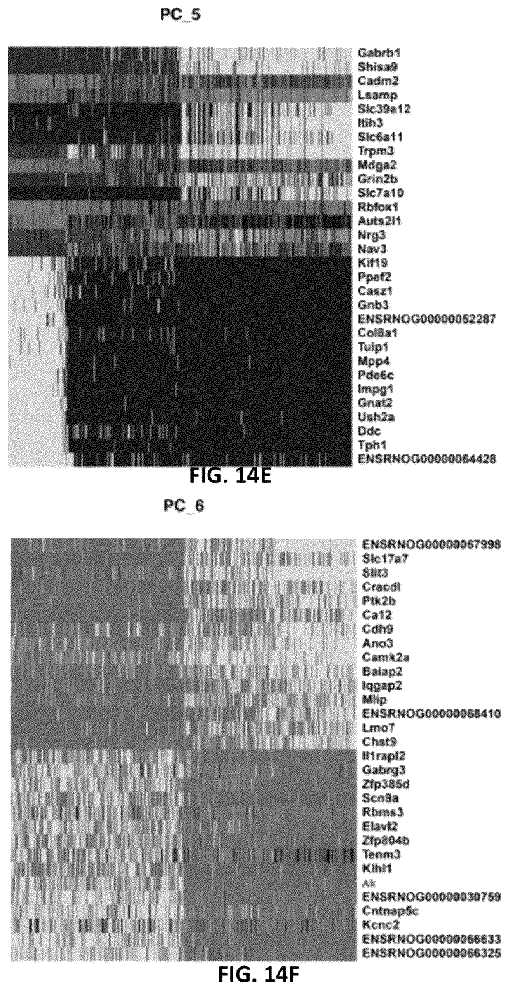

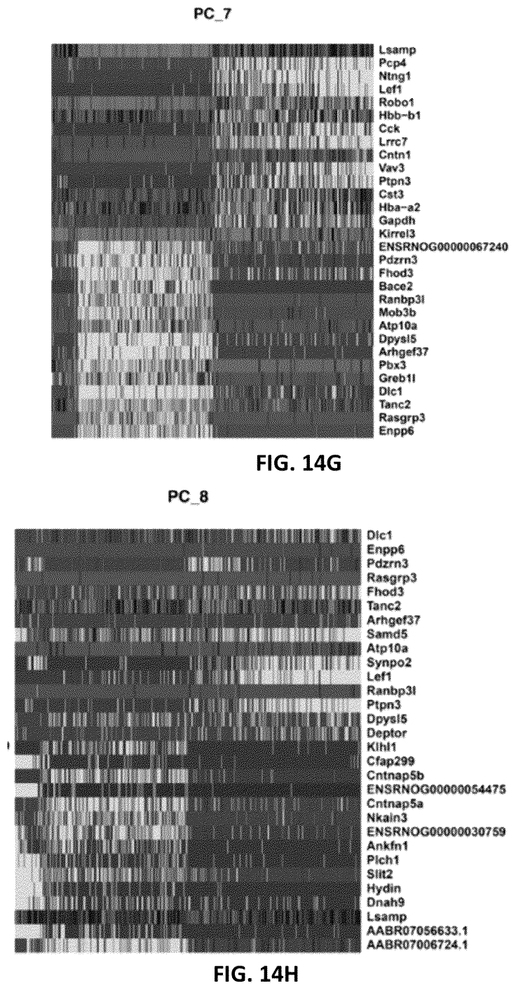

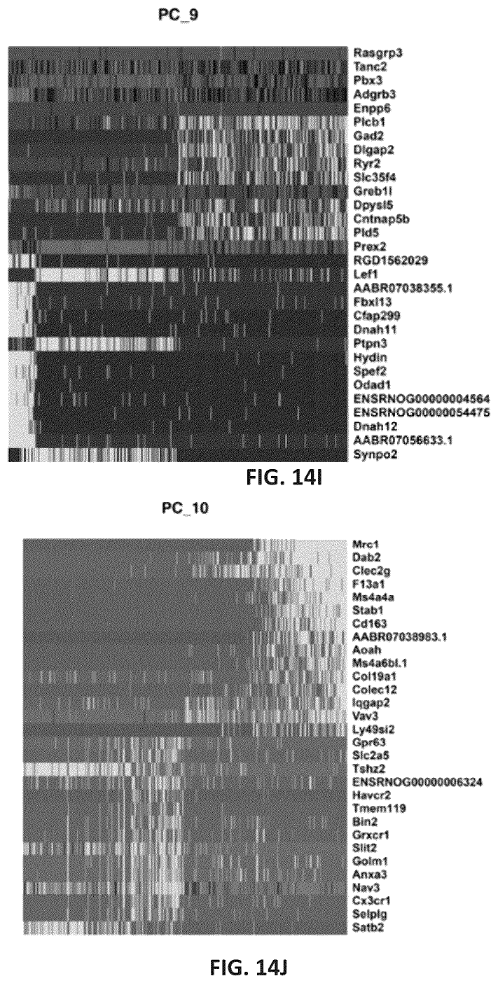

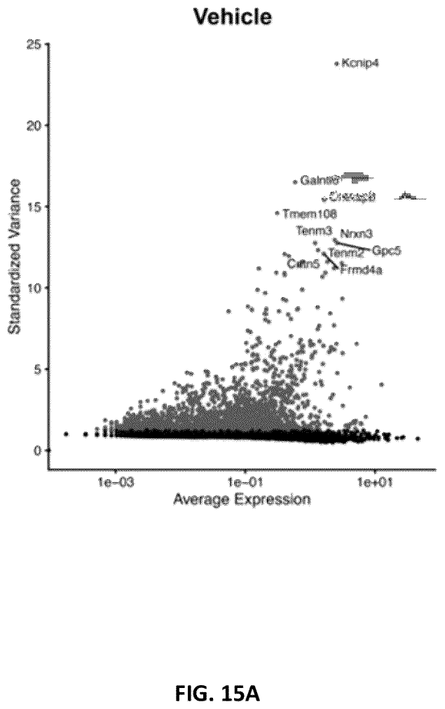

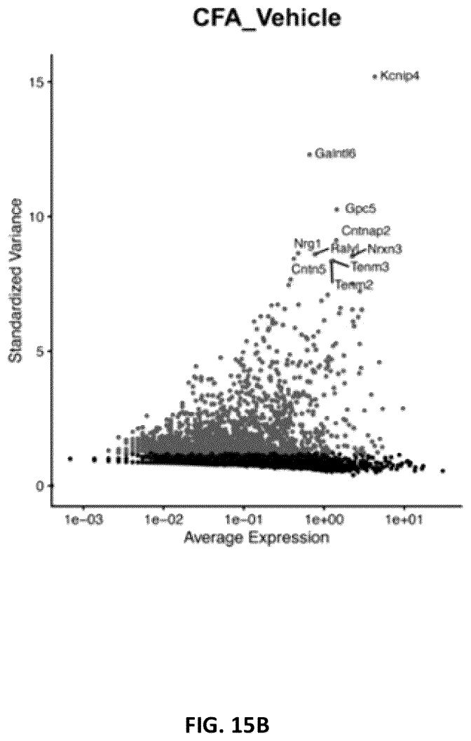

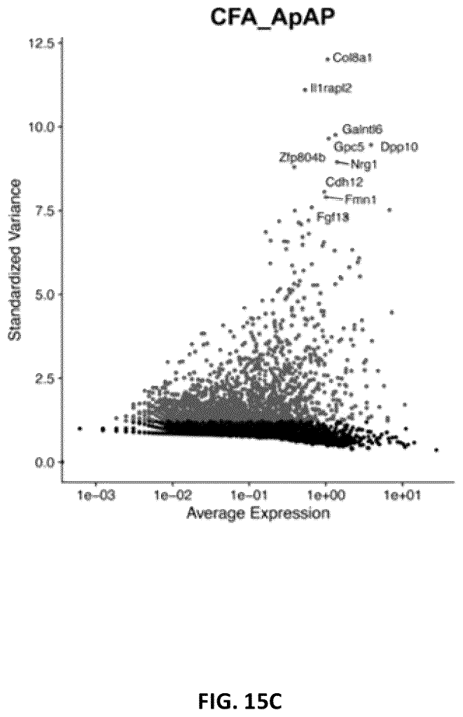

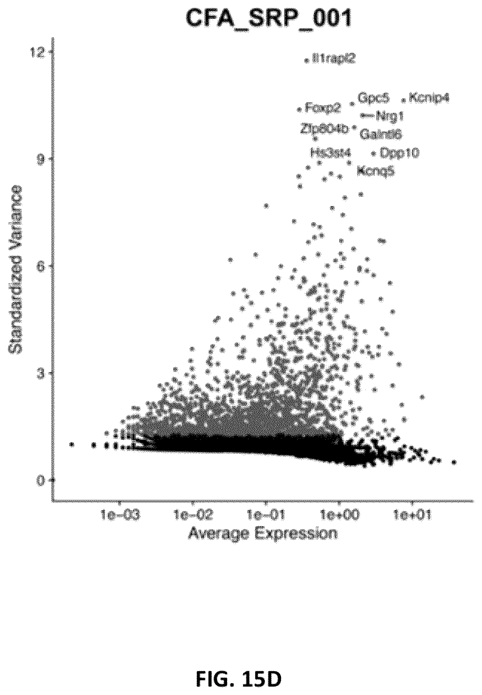

Dimensionality plots from principal component analysis (PCA) show the top 30 differentially expressed genes in principal clusters 1-10 selected based on the elbow plot (FIG. 14). Highly variable genes were identified in each group, and the top 10 from each group were visualized (FIG. 15).

### Differential Gene Expression of Neurons and Other Cell Clusters of PAG

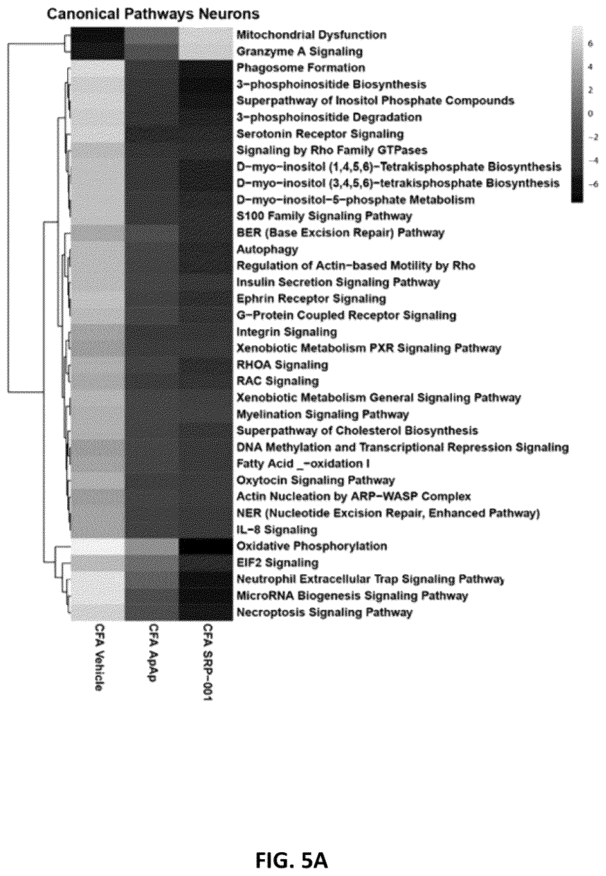

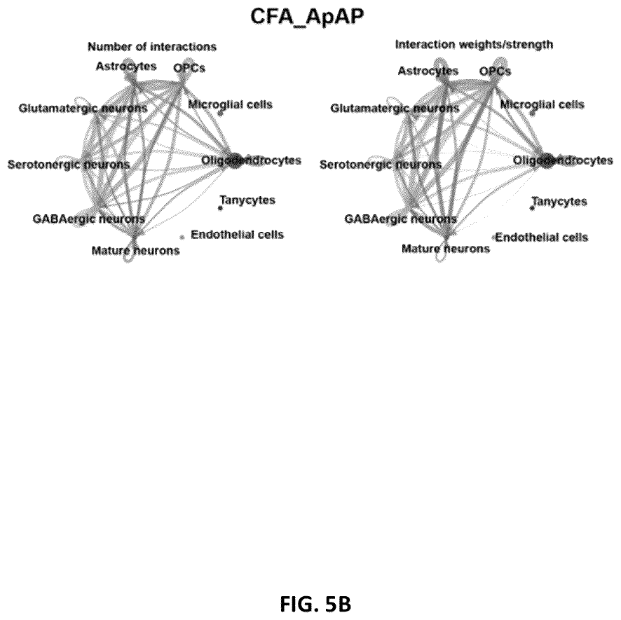

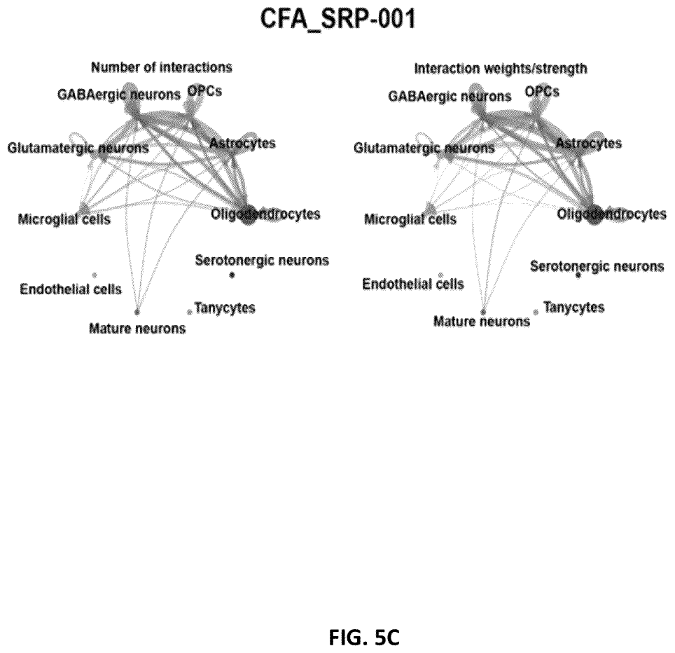

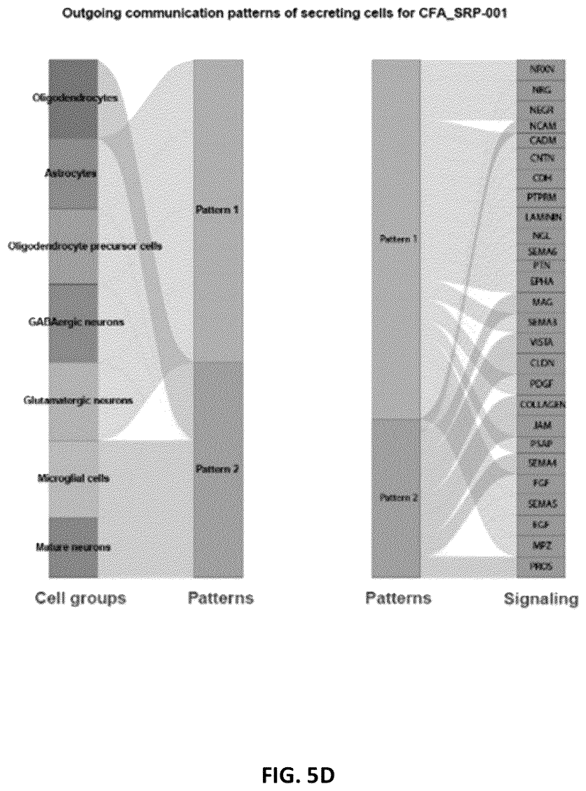

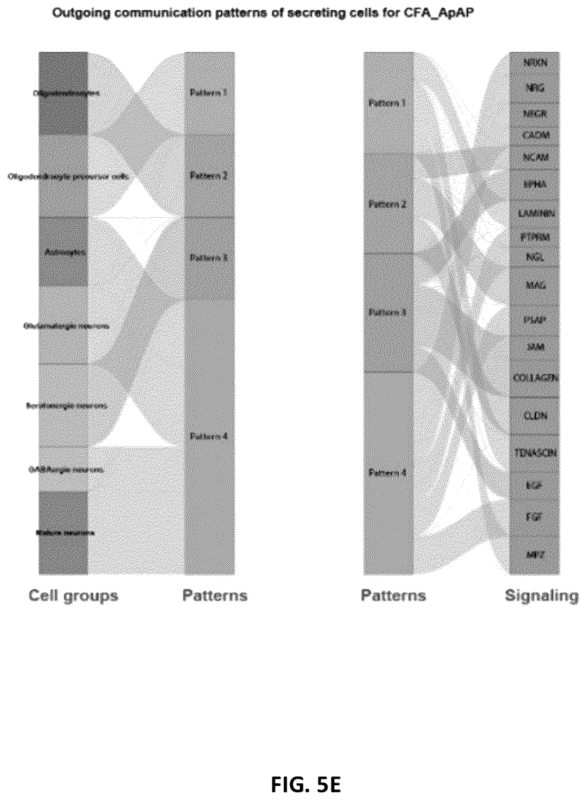

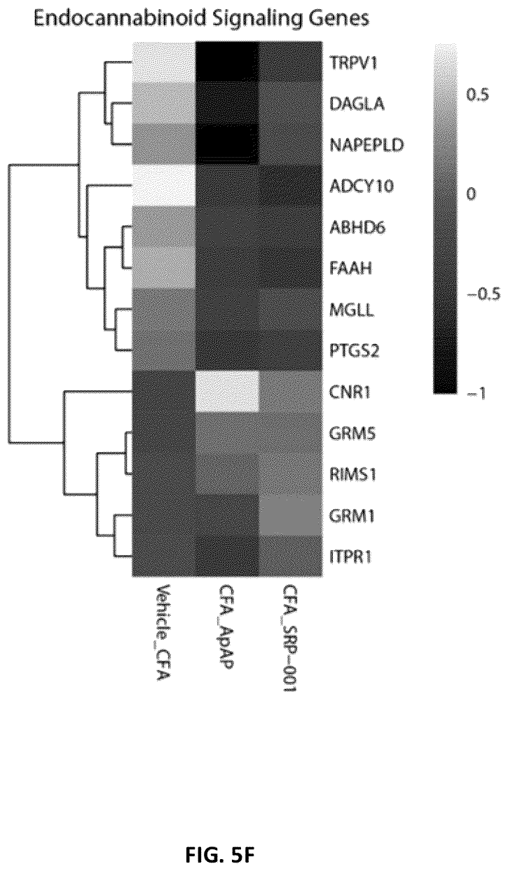

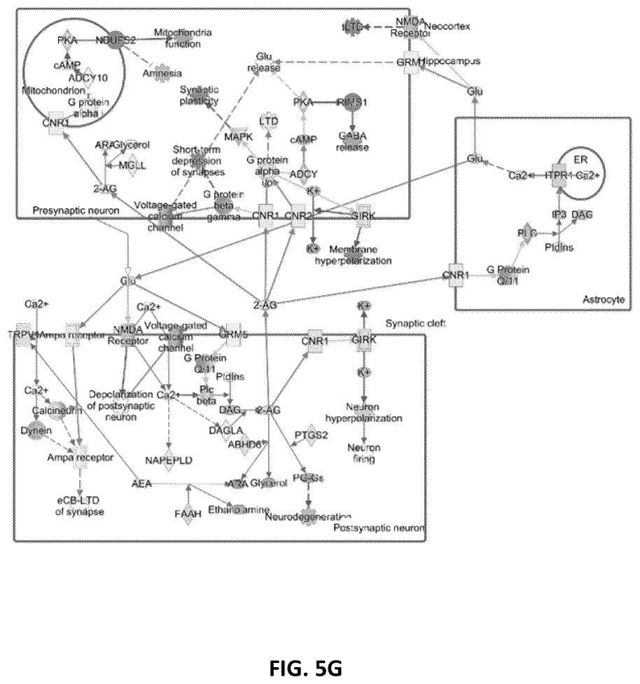

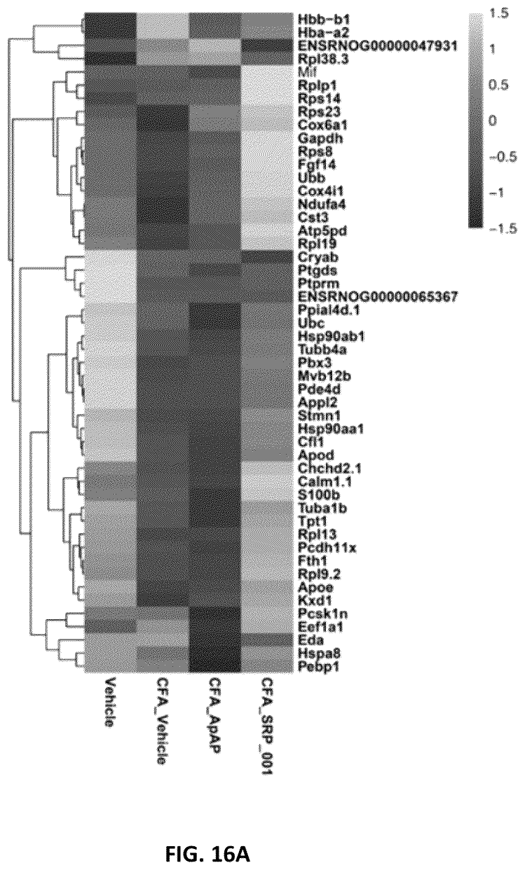

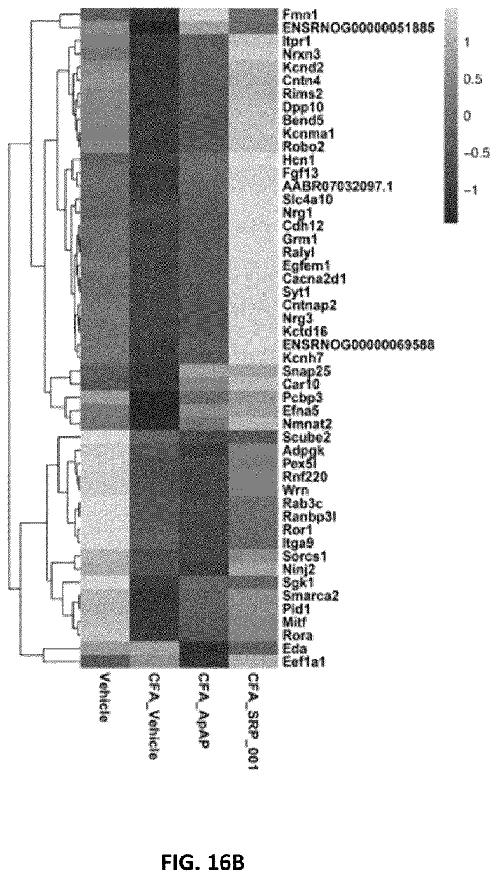

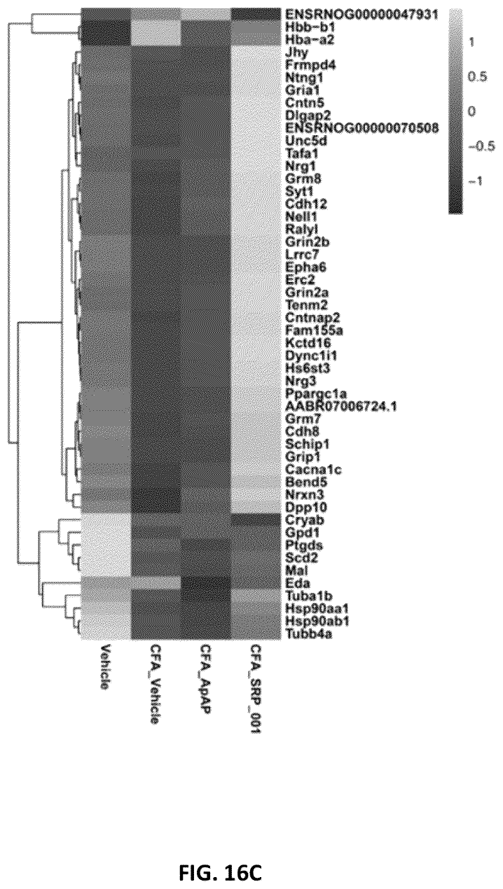

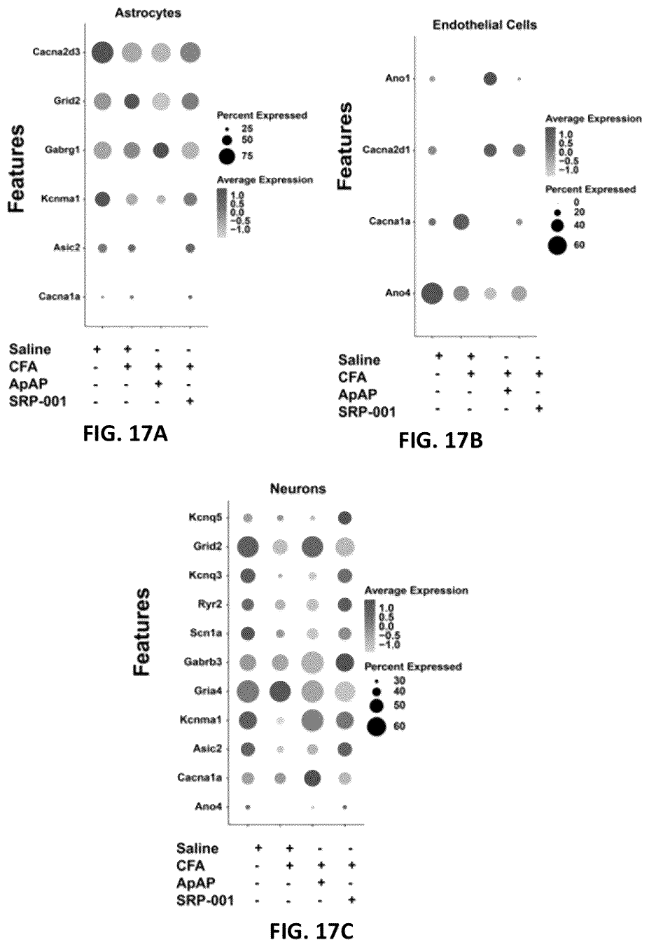

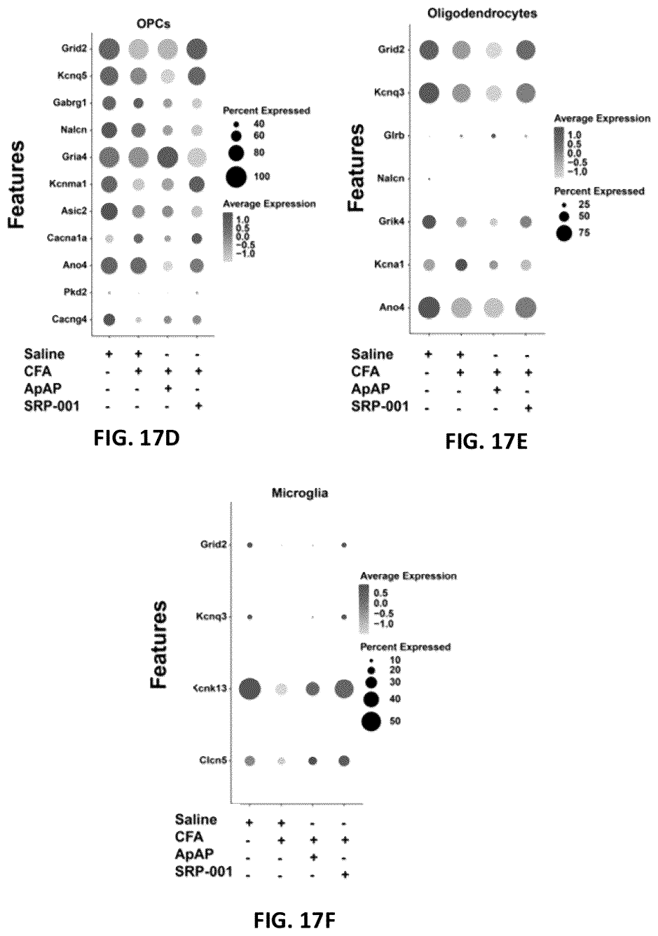

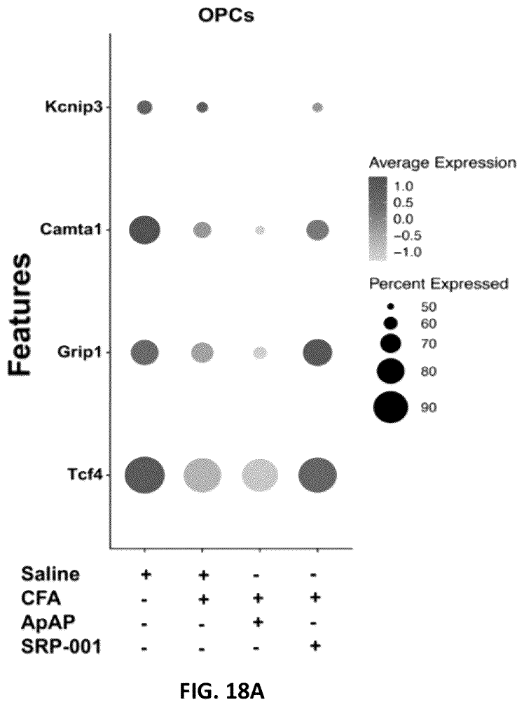

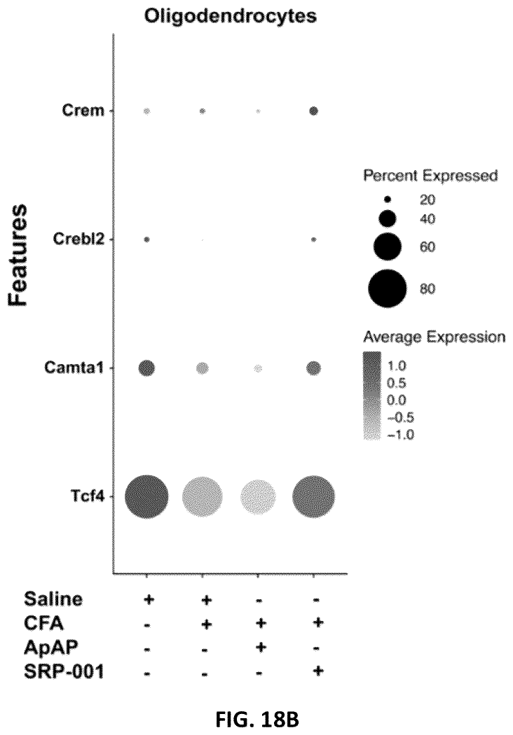

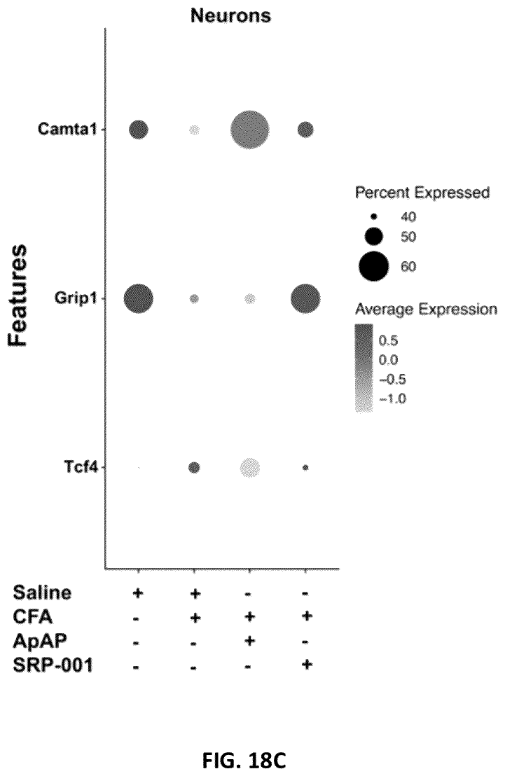

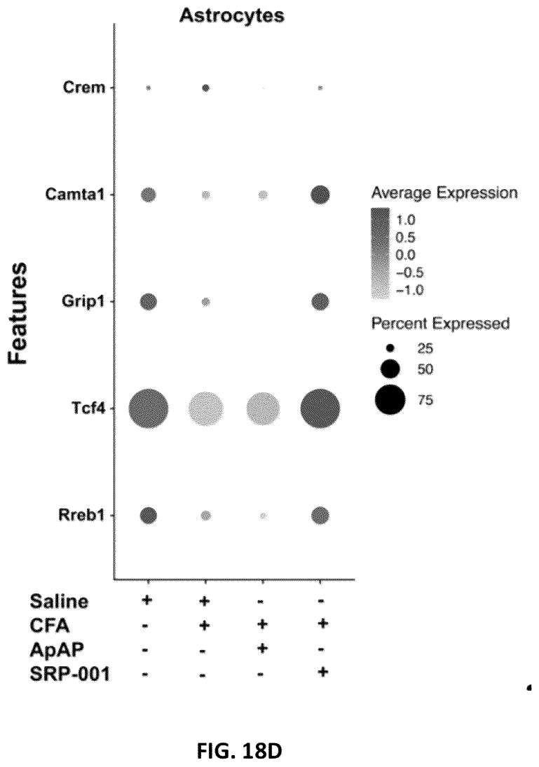

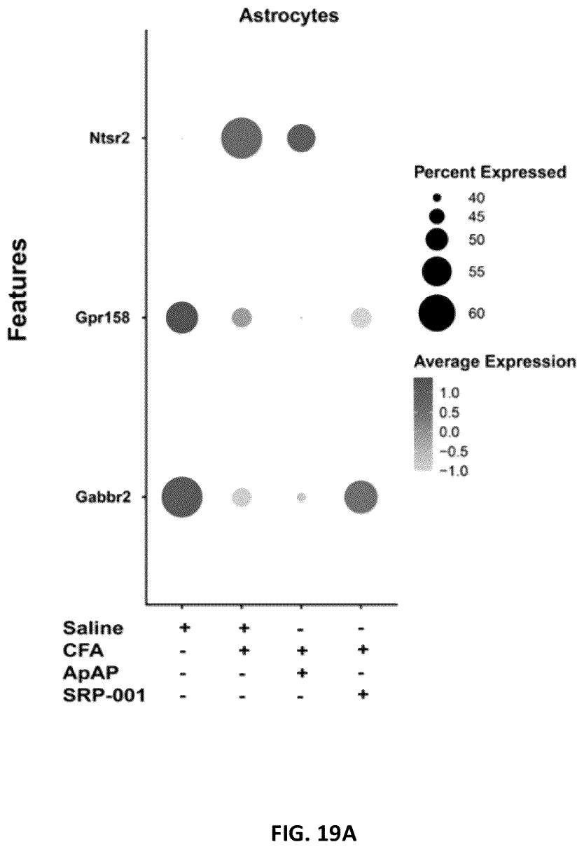

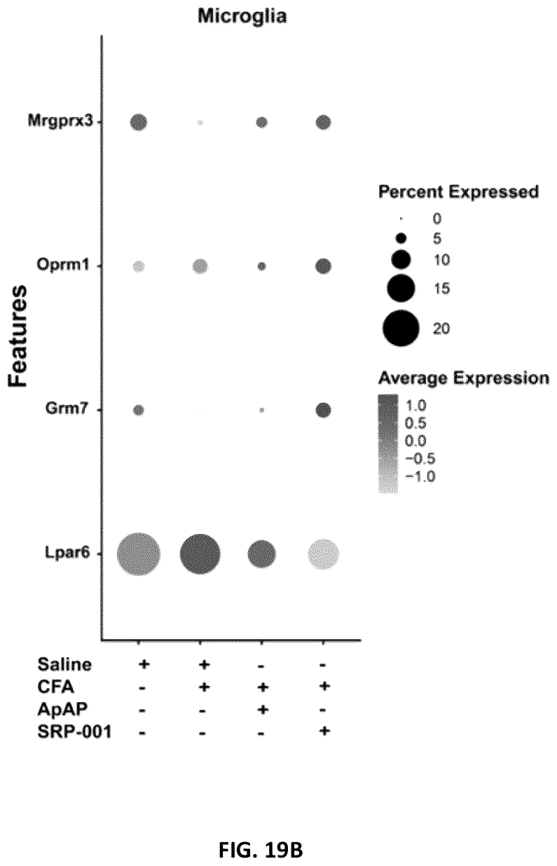

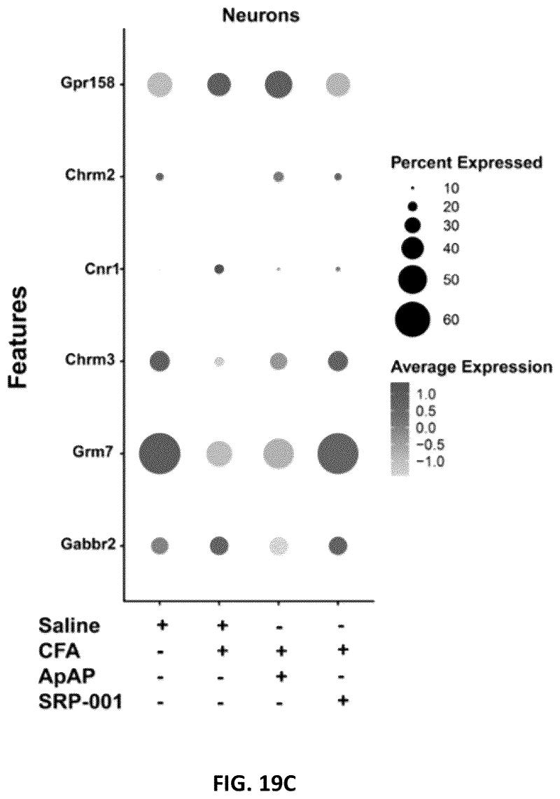

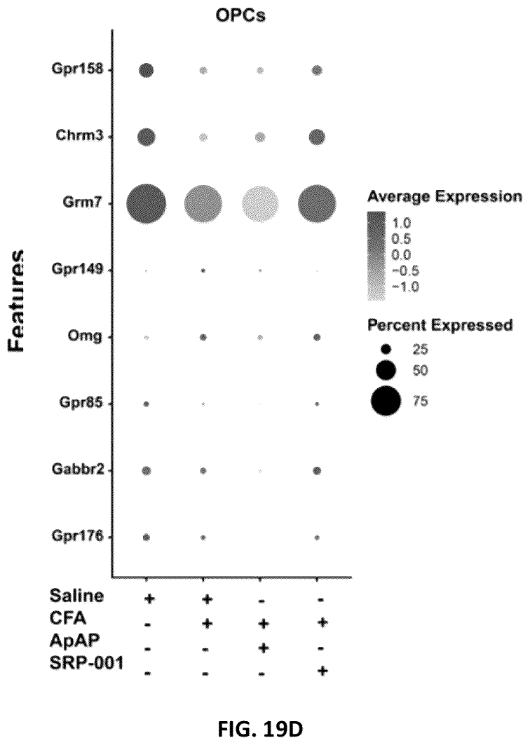

Differential expression analysis between groups Vehicle vs. CFA_Vehicle, CFA_Vehicle vs. CFA_ApAP, and CFA_Vehicle vs. CFA_SRP-001 was performed using DESeq2, and the top 50 DE genes are shown in heatmaps illustrating that both ApAP and SRP-001 are predicted to regulate similar gene regulatory pathways and that SRP-001 changes the effects based on fold regulation prediction scores (FIG. 16). A comparative analysis of the top 40 canonical pathways across neuronal clusters was conducted, generating a heatmap for visual representation of the following sample comparisons: Vehicle vs CFA_Vehicle, CFA_Vehicle vs CFA_ApAP, and CFA_ApAP vs CFA_SRP-001 (FIG. 5A). Pathways depicted in the heatmap were identified using Qiagen's Ingenuity Pathway Analysis (IPA) tool, applied to a normalized matrix of differentially expressed genes between the neuronal clusters under comparison. The heatmap reveals an upregulation of genes impacting cellular signaling pathways when comparing CFA_Vehicle to Vehicle. Conversely, both CFA_ApAP and CFA_SRP-001 exhibit downregulation of these upregulated pathways when compared to CFA_Vehicle, with a more pronounced downregulation observed for CFA_SRP-001. We also investigated a compendium of known and validated pain-related genes and defined the differential expression of these genes across sample comparisons—CFA_Vehicle vs Vehicle, CFA_ApAP vs CFA_Vehicle and CFA_SRP_001 vs CFA_Vehicle and also across different cell clusters between these samples. Dot plots show that several of these genes are differentially expressed across samples and similar among CFA_ApAP and CFA_SRP-001 treatment groups. Genes were separated based on classification as ion channel-, G-protein coupled receptor-, or transcription factor-associated FIGS. 17, 18, 19).

### Comparing Unbiassed Signaling Pathways and Networks Between Samples to Discern the MOA and Cellular Signaling Between SRP-001 and ApAP

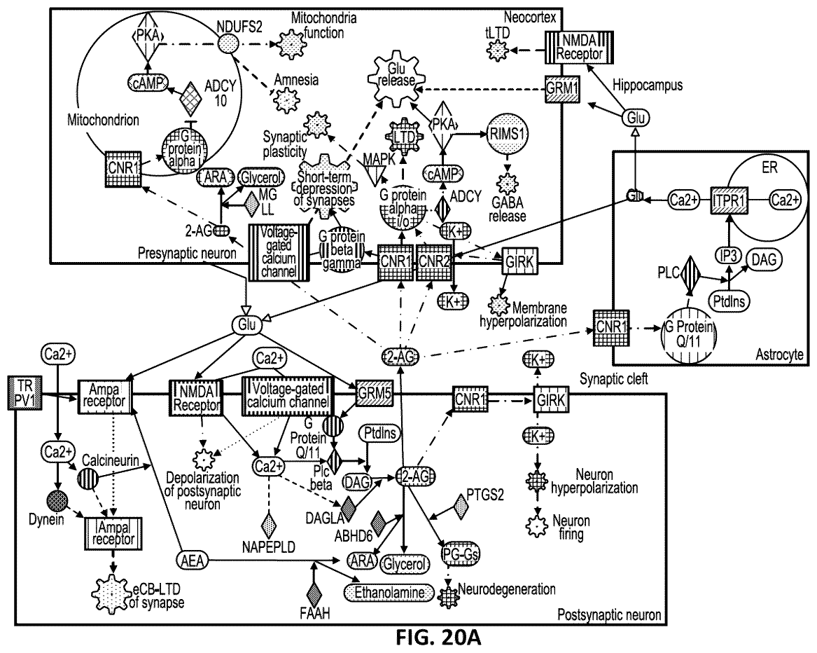

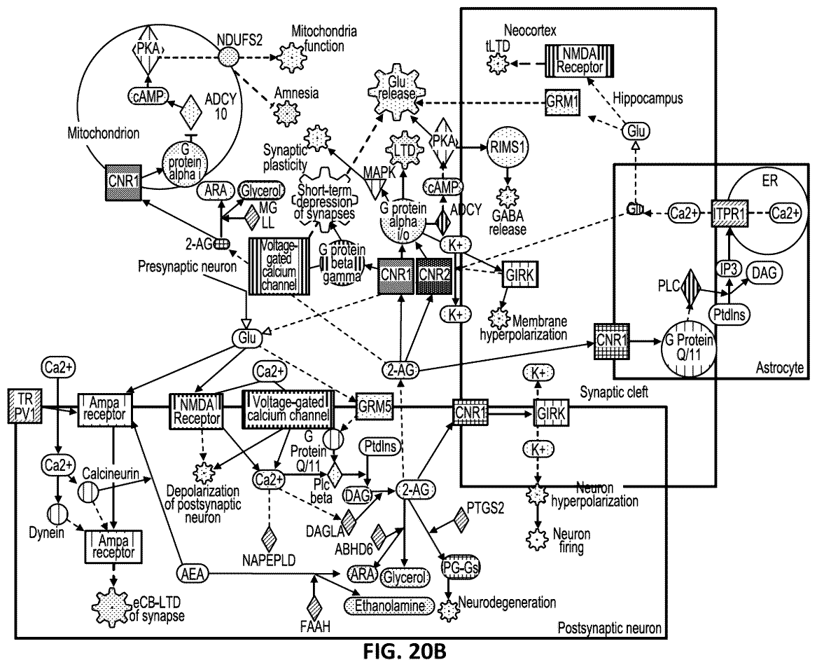

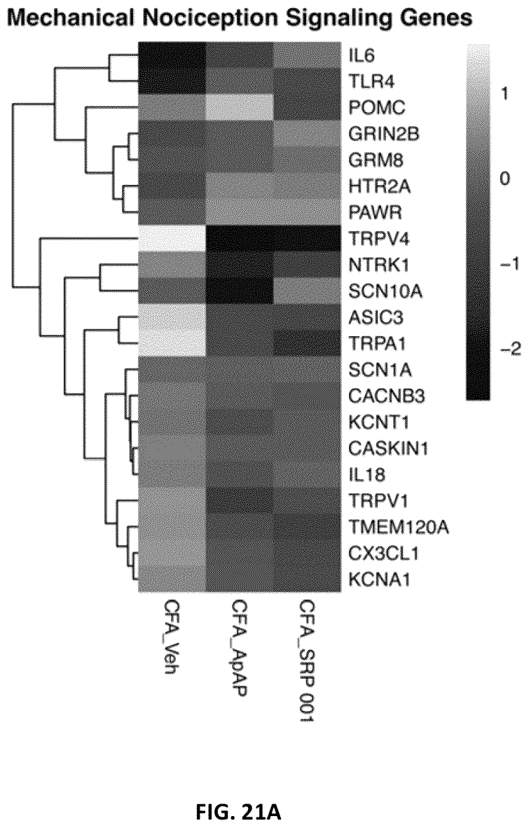

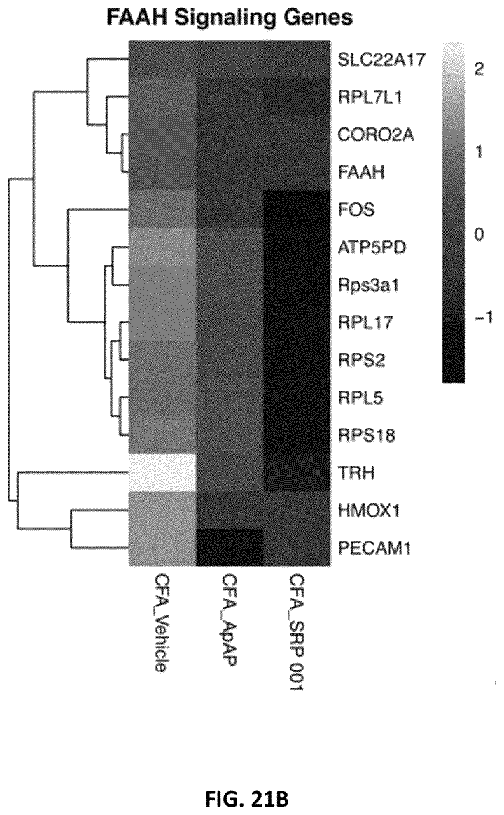

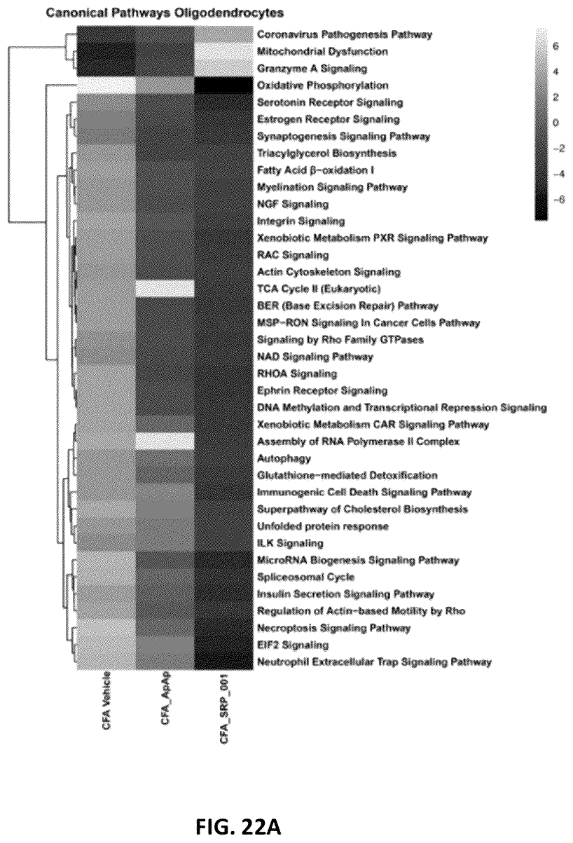

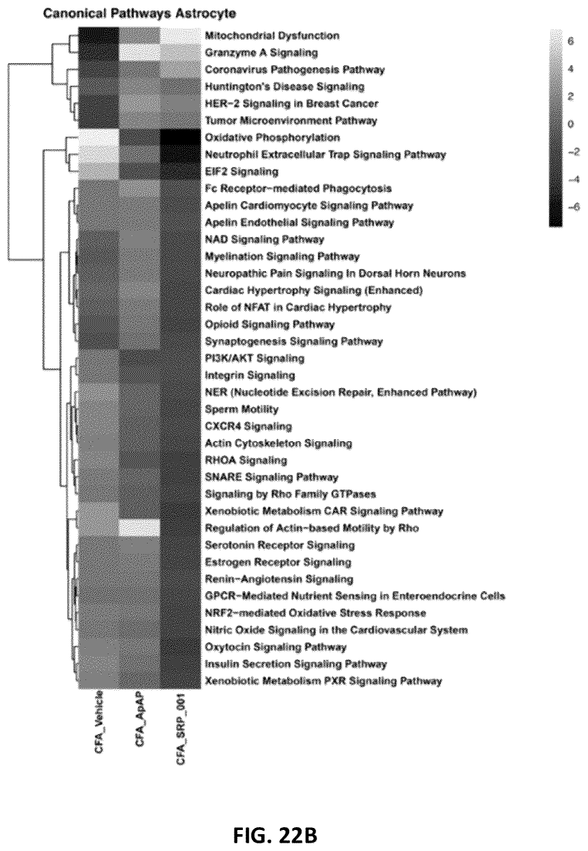

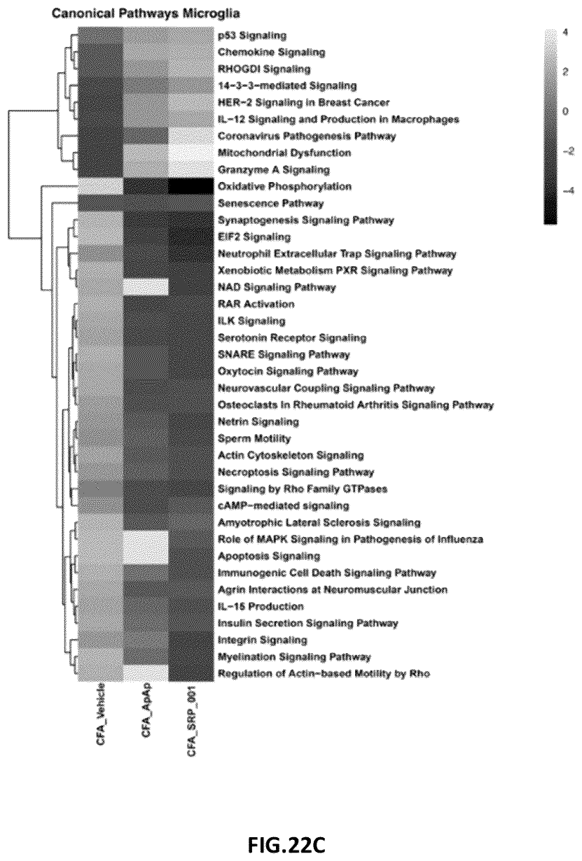

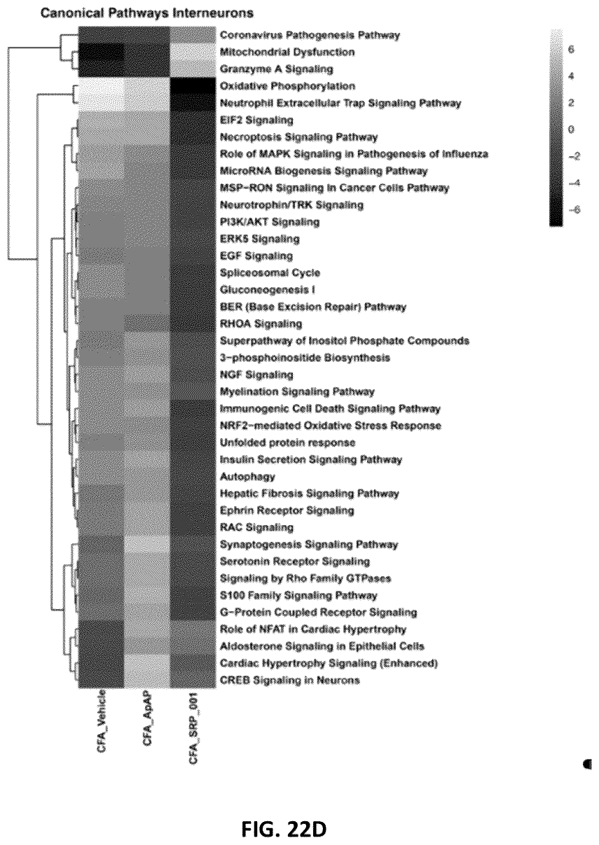

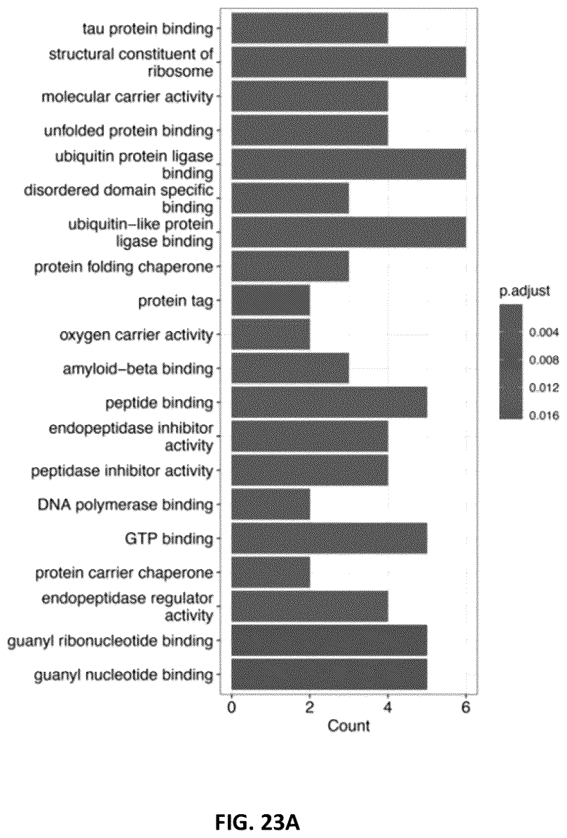

Next, CellChat analysis was employed to computationally model anticipated cell-cell interactions, utilizing single-cell data to elucidate potential mechanistic parallels between the CFA_ApAP and CFA_SRP-001 groups. The cell-cell interaction maps demonstrating number and strength of interactions is shown in FIG. 5B-5C. River plots for outgoing communication patterns of secreting cells and the corresponding pathways were determined for CFA_ApAP and CFA_SRP-001 from this analysis as well (FIG. 5D-E). Next, we explored well-defined ApAP-related target networks and signaling pathways: endocannabinoids; mechanical nociception; and fatty acid amide hydrolase (FAAH) using Qiagen's IPA analysis tool (FIGS. 5F-5G and FIGS. 20-21). These analyses demonstrate that the gene expression profiles for ApAP-targeted pain-related genes in these pathways are activated and inhibited in a similar manner between SRP-001 and ApAP. Heatmaps were generated from exported IPA tables containing the pathways' analysis values and corresponding gene symbols. This highlights the similarities in gene regulatory activity of endocannabinoid signaling genes TPRV1, ADCY10, ABHD6, FAAH, PTGS2, CNR1, GRM5, RIMS1, GRM1, and ITPR1 between ApAP and SRP-001 (FIG. 5F). Genes implicated in mechanical nociception—including TRPV1, NTRK1, ASIC3, TRPA1, TMEM120A, CX3CL1, KCNA1, IL6, GRIN2B, GRM8, PAWR, KCNT1, CASKIN1, and IL18—exhibit comparable modulatory efficacy between SRP-001 and ApAP, as detailed in FIG. 21A). Similarly, FAAH signaling genes were more significantly regulated by SRP-001 than ApAP, specifically: FOS, ATPSPD, Rps3a1, RPL17, RPS2, RPL5, RPS18, and TRH (FIG. 21B). Moreover, the expression of these pain-related genes is higher in SRP-001 as compared to ApAP. In summary, canonical pathway analysis with predicted inhibition or activation shown in heatmaps highlights the similarities between CFA_ApAP and CFA_SRP-001 in different cell clusters—neurons (FIG. 5A), oligodendrocytes, astrocytes, microglia, and interneurons (FIG. 22A-22D) respectively.

### Genotoxicity, Safety Pharmacology, and In Vivo Non-Clinical Toxicology

To transition SRP-001 into the clinic for human use, a full set of in vivo and in vitro genotoxicity studies were performed to assess the potential genotoxicity of SRP-001. This included a full battery of International Conference on Harmonization (ICH) compliant studies, including in vitro Ames and mammalian chromosomal aberration assays, in vivo mammalian micronucleus and Comet assays indicating that SRP-001 is not genotoxic. Further, SRP-001 has no effects on pulmonary function or cardio-telemetry and is non-cardiotoxic (Table 1)

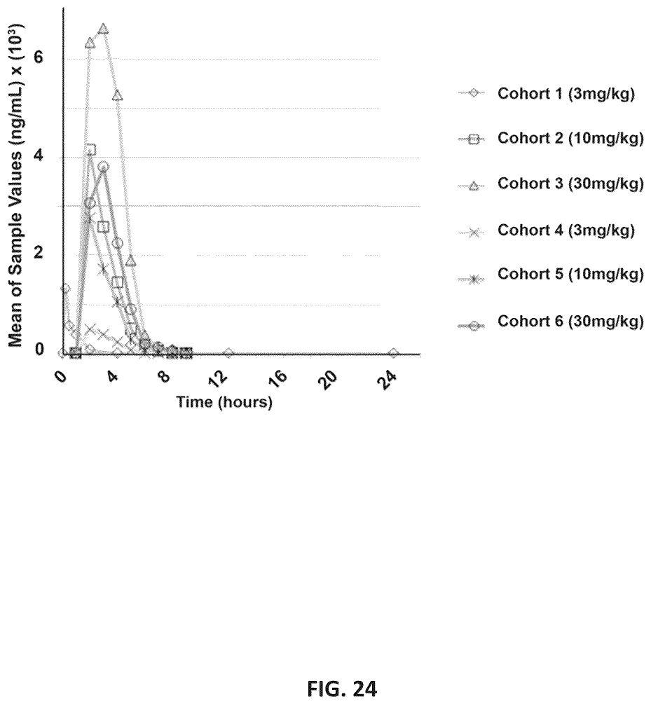

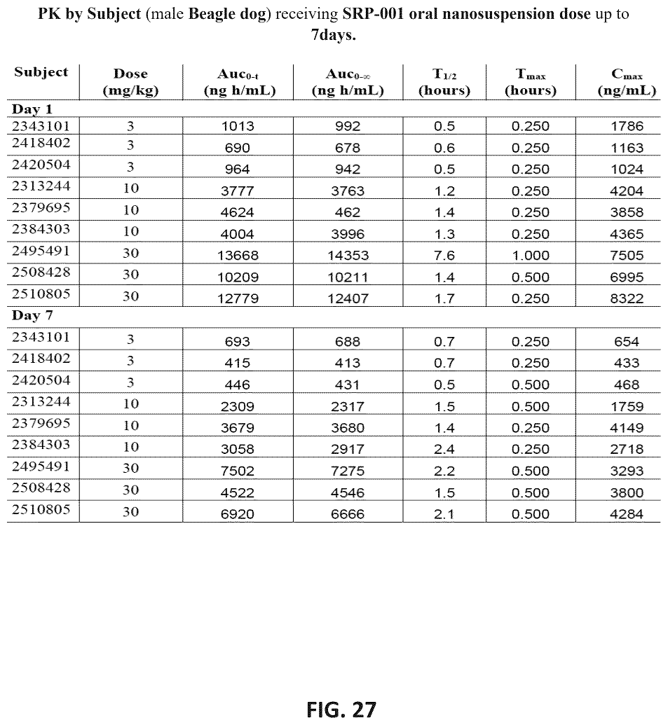

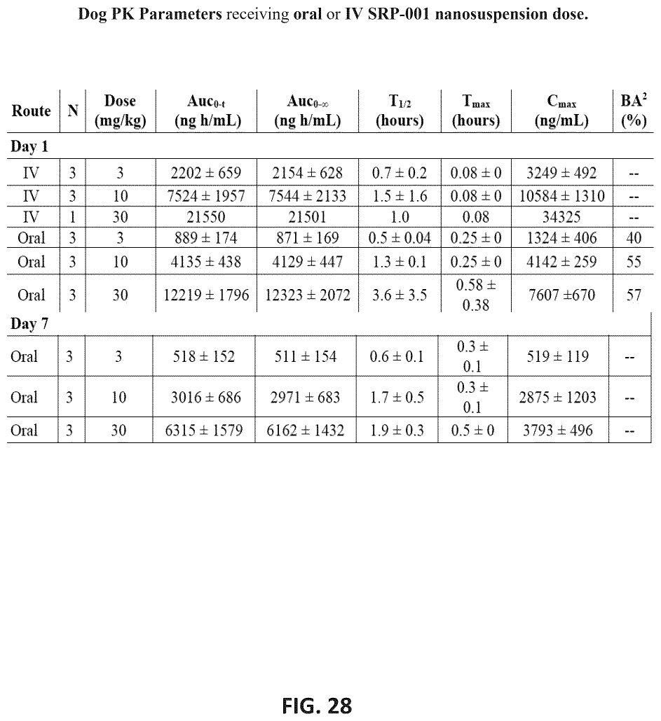

To determine the appropriate doses, maximum tolerated dose studies (non-GLP) were conducted prior to the definitive GLP systemic toxicity studies. SRP-001 was evaluated by a full battery of GLP-compliant toxicity studies. Systemic toxicity was evaluated in Sprague-Dawley rats and Beagle dogs by oral administration over 28 days (FIG. 24 and FIGS. 27-28, Table 2). Importantly, no treatment-related adverse effects were seen during 28 days of daily oral doses of SRP-001 in rats at doses up to 1500 mg/kg/day or in dogs at doses up to 330 mg/kg/day. Single doses of up to 2000 mg/kg in the rat and 1200 mg/kg in the dog of SRP-001 did not result in any mortality, whereas all ApAP-treated mice expired following a 900 mg/kg dose.

### Discussion

The use of opioids for pain management is linked to opioid use disorder (OUD) due to their abuse potential. In 2021, the U.S. saw 107,000 drug overdose deaths with over 80,000 attributed to opioids. Many of these cases originated from prescription opioid misuse for pain management, leading to opioid use disorder (OUD) and even transitioning to heroin use. According to the National Institute on Drug Abuse (NIDA), around 21-29% of patients prescribed opioids for chronic pain misuse them, and about 8-12% develop OUD. Although ApAP is most used to treat short-term pain, hepatotoxicity is a risk from overise, and it is the most common cause of fulminant hepatic failure in patients with compromised liver function. And NSAID overuse carries risks of GI bleeding and nephrotoxicity. The high levels of opioid addiction, misuse, and overdose and the toxicity profiles of currently available pain medications underscore the need for safe, effective, non-opioid pain medications.

While ApAP is generally deemed safe within prescribed dosages, it is crucial to acknowledge that certain populations—specifically, individuals with liver dysfunction or regular alcohol consumption—encounter a reduced therapeutic-to-toxic dose margin. The U.S. FDA has taken steps to mitigate this risk, further underscoring the need for developing safer alternatives. Owing to ApAP's hepatotoxicity, we characterized SRP-001 from a library of ApAP analogs that lacked bepatotoxicity and exhibited antinociception and antipyresis. SRP-001 is not hepatotoxic because it does not generate NAPQI or disrupt hepatic tight junctions, which are hallmarks of ApAP hepatotoxicity. An equimolar dose-response toxicity analysis comparing the mortality effects of ApAP and SRP-001 in CD-1 male mice revealed that while the placebo and SRP-001 groups showed no mortality across all dosages, the ApAP groups exhibited a dose-dependent increase in mortality. Employing the Cox proportional hazards regression model, we found significant differences in mortality rates between the ApAP and SRP-001 groups, particularly at higher ApAP concentrations. Kaplan-Meier analysis further supported these findings, illustrating a clear dose-response increase in mortality with ApAP reaching 70% within 72 hours, in contrast to SRP-001, which showed no mortality in the treated animals.

For the in vivo antinociception studies, we used reliable, reproducible animal models and assays of acute and chronic pain used in the preclinical testing of novel and translationally relevant analgesics. CFA is a chronic inflammatory pain model, and eVF and Hargreaves are the detection methods to measure mechanical (eVF) and thermal (Hargreaves) nociceptive sensitivity, respectively. Taken together, these pain models suggest that SRP-001 produces efficacious antinociception across various acute and chronic pain conditions, with a larger therapeutic index than ApAP and broad efficacy. As an extension of the current studies, future preclinical and clinical work should determine the efficacy of SRP-001 in reducing the negative affective dimension of pain as well as carefully screen for any undesirable side effects.

Despite being available since the 1950s, ApAP's analgesia MOA remains to be fully discerned. Increasing evidence, however, strongly supports that AM404 is fundamental to its analgesia. In the liver, ApAP is converted to p-aminophenol, which, in turn, is converted by FAAH in the brain in the presence of arachidonic acid to make AM404. Current understanding of ApAP's MOA analgesia is through AM404 via the endogenous cannabinoids through CB1 receptors and activation of the TRPV1 channel-receptor signaling in the midbrain PAG. Here, we demonstrate after ip-injection of D₁₀-SRP-001 in rat brain and also that SRP-001 produces more AM404 than ApAP in the PAG where FAAH/CB1/TRPV1 channel triad co-locates. Beyond this, the central mechanisms for ApAP's analgesia are still unclear.

By examining FAAH gene expression interaction networks, we found similarities in gene modulation related to pain signaling between ApAP and SRP-001. Downregulation of FAAH, CORO2A, and RPL7L1 measured directly from scRNAseq was observed with both ApAP and SRP-001. FAAH is a key enzyme in pain signaling by modulating endocannabinoid levels and lipid-based signaling mediators that regulate physiological processes, including pain perception. The primary function of FAAH is degradation of anandamide (AEA), a main endocannabinoid involved in pain modulation. The role of FAAH in pain signaling has been extensively studied, and it has been found that inhibition of FAAH activity can lead to an increase in AEA levels, reducing pain perception. This process is mediated by the activation of CB1 and CB2 receptors. In addition to AEA, FAAH also degrades other bioactive fatty acid amides, such as oleamide and palmitoylethanolamide, which possess antinociceptive properties. Directly measured expression from scRNAseq data shows decreased expression of FAAH and genes related to the molecular function of FAAH by SRP-001 and ApAP, indicating that the bioactivity of SRP-001 produces antinociception through FAAH inhibition.

Mechanical nociception gene networks displaying genes that are involved in this biological function also demonstrate the similarities between gene modulatory mechanisms of SRP-001 and ApAP. Activation of vlPAG-located TRPV1 channels, which are expressed on glutamatergic neurons, is required by ApAP to exert analgesic effects via AM404. Activation of these receptors produces analgesic effects through increasing glutamate release to act on mGlu₅ receptors. ApAP has recently been shown to increase glutamate and GABA levels in the presence of an acute pain stimulus in a FAAH-dependent manner. Ion channels present another key target for pain modulation due to their localization in primary sensory neurons. Various receptors and ion channels play a critical role in determining neural excitability; PAG pain transmission through descending facilitatory effect can potentially contribute to the development and maintenance of hyperalgesia, which is dependent upon neuron network activity. Previously, calcium and sodium gated ion channels were the focus of pain signaling, but recently, potassium channels have emerged as a potential target for novel analgesics. Potassium channels are necessary to sustain resting membrane potential and repolarizing neurons after an action potential; thus, several potassium channels have been implicated in pain modulation in the PAG area, including the ATP-sensitive potassium (K-ATP) channels, the calcium-activated potassium (KCa) channels, and the two-pore domain potassium (K2P) channels. Concerted activation of proteins, including ion channels, can lead to peripheral sensitization, thus, inhibition of ion channel activity can reduce neuronal excitability and prevent this peripheral sensitization. Modulation of mechanical nociceptive genes TRPV4, TRPV1, ASICS3, TRPA1, KCNA1, and KCNT1 by SRP-001, provides an early indication of broad ion channel inhibition and potential MOA.

Despite the widely recognized need for safer and more effective pain medications is widely recognized, particularly considering the opioid epidemic in the U.S., the lack of innovation in this space has left patients with limited options. Backpedaling on 2016 guidelines urging physicians to restrict the use of opioids for moderate-to-severe acute and chronic pain, the Centers for Disease Control and Prevention recently updated its Clinical Practice Guidelines for Prescribing Opioids. The high failure rate of novel pain therapeutics in clinical studies compared to other fields in medicine, the limited availability of investor funding due to this increased risk, and the poor appetite for pain therapeutics among pharmaceutical companies are causes for marginal innovation toward safer and effective pain therapeutics. In order to reduce the developmental risk, these considerations emphasize the significance of determining whether a novel pain therapeutic candidate alleviates pain via clinically validated MOA. Thorough pre-clinical safety evaluations, AM404 and transcriptomic studies showcasing a validated, clinically established MOA similar to ApAP, combined with Phase 1 safety data, collectively suggest that advancing the development of SRP-001 could potentially provide a safe and effective pain relief option for acute and chronic pain in humans, characterized by a large therapeutic window.

### Limitations

In the present study, we demonstrate a well-matched alignment between behavioral assays and the neurobiological region of focus, specifically the midbrain. Upcoming research will integrate pain-avoidance assays in rats, developed in-house, to more accurately quantify the motivational aspects of pain. These assays aim to capture higher limbic elements, such as cortical regions, associated with pain and pain-related negative affective-like behavior. Also, analgesic compounds such as opioids and alcohol consistently elevate paw withdrawal thresholds above baseline, corroborated by our findings with ApAP. For SRP-001, no overt alterations in locomotor activity or motor incoordination are evident. Future investigations will rigorously assess potential motor and other side effects of SRP-001 in both pre-clinical and human models. Finally, although pre-clinical data shows SRP-001's efficacy in three antinociception assays and we elucidate its mechanism of action, only upcoming Phase 2 trials can confirm its clinical analgesic efficacy.

### Code Availability

No custom codes were used for data analysis. All data analyses were performed using freely available open-source software for bioinformatics from Bioconductor and GitHub.

### EXPERIMENTAL

We recently synthesized a library of 2-(benzenesulfonamide)-N-(4-hydroxyphenyl) acetamide analgesics in search of non-hepatotoxic ApAP analogs. From this library, SRP-001 was chemically synthesized using readily available commercial analytical grade reagents of highest quality, which were purchased and used without further purification. Melting points were determined in open capillary tubes on a Stuart Scientific SMP3 melting point apparatus. Mercury VX-300, Bruker BioSpin GmbH 400 MHz, or Varian Unity 500 MHz spectrometers were used to record ¹H and ¹³C NMR spectra at room temperature. Chemical shifts are given in ppm (δ) downfield from TMS. Coupling constants (J) are in hertz (Hz), and signals are described as follows: s, singlet; d, doublet; t, triplet; br, broad; m, multiplet. Mestrenova 12.0.4 software was used to analyze the NMR FIDs. Purity of the products was determined by chromatographic analysis using an Agilent 1200 with diode array detector and Agilent 1100 MSD-Q mass detector (Column C18 Luna, 100 mm×4.6 mm×3 μm. Mobile phase A: water with 0.1% of formic acid. Mobile phase B: MeOH with 0.1% of formic acid. Elution gradient: 5% B to 100% B in 20 min. Flow: 1 mL/min with Split 1:2 for MS detection. UV wavelengths: 214, 254 nm. Mass Detection: Scan 50-1000 m/z.). N-(4-Hydroxyphenyl)-2-(1,1,3-trioxo-1,2-benzothiazol-2-yl) acetamide 1 was synthesized as previously described.

### N,N-diethyl-2-[[2-(4-hydroxyanilino)-2-oxo-ethyl]sulfamoyl]benzamide (SRP-001)

To N-(4-hydroxyphenyl)-2-(1,1,3-trioxo-1,2-benzothiazol-2-yl) acetamide 1 (0.165 g, 0.496 mmol), a solution of diethylamine (0.154 mL, 1.5 mmol) in acetonitrile (3 mL) was added. The mixture was refluxed for 16 h. Evaporation under reduced pressure gave a brown residue, which was purified by chromatography [silica gel60 F254, 70-200 mm, ethyl acetate:hexane (6:4)], followed by crystallization from ethyl acetate:hexane, yielding SRP-001 as a white solid (0.132 g, 66%). mp 171-172° C.; ¹H NMR (400 MHz; DMSO-d₆; Me₄Si) δ(ppm): 9.69 (s, 1H), 9.18 (s, 1H), 7.91 (dd, J=7.6, 1.5 Hz, 1H), 7.73-7.54 (m, 2H), 7.46 (brs, 1H), 7.43 (dd, J=7.4, 1.5 Hz, 1H), 7.22 (d, J=8.8 Hz, 2H), 6.64 (d, J=8.8 Hz, 2H), 3.77-3.46 (m, 3H), 3.38-3.28 (m, 1H), 3.16-2.95 (m, 2H), 1.17 (t, J=7.0 Hz, 3H), 1.02 (t, J=7.1 Hz, 3H); ¹³C NMR (101 MHz, DMSO-d₆) δ (ppm): 168.4, 165.4, 153.5, 136.3, 135.3, 132.8, 130.0, 129.3, 128.6, 127.3, 121.0 (2C), 115.0 (2C), 45.6, 42.7, 38.3, 13.1, 11.9; purity by HPLC 99.1%; MS (ESI⁺) (m/z) 406.20 (MH⁺). Anal. Calcd. for C₁₉H₂₃N₃O₅S: % C 56.28, % H 5.72, % N 10.36, % S 7.91. Found: % C 56.73, % H 5.85, % N 10.56, % S 8.17.

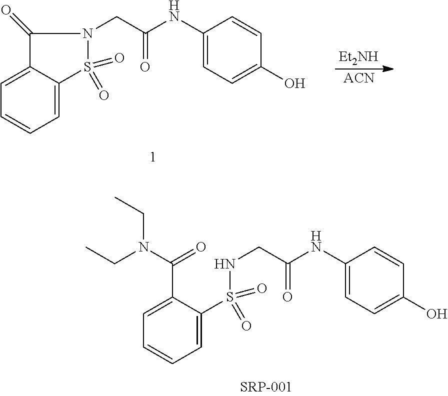

### Serum Isolation for NAPQI Identification, Characterization of Novel Benzoquinoimine, and Liver Function Tests (LFTs)

Male CD1 mice were fasted overnight for 15 h and dosed with ApAP or SRP-001 or vehicle (0.9% saline) at doses of 600 mg/kg administered via per os (PO) injections with an administration volume of 10 mL/kg body weight. After drug administration, animals were returned to their respective cages and maintained with food and water provided ad libitum for the next 12 h. Animals were then euthanized under 5% isoflurane anesthesia after 12 h, and whole blood samples were collected transcardially in sterile microcentrifuge tubes without anti-coagulants. Whole blood samples were stored at room temperature (25° C.) for 30 min, allowing them to coagulate, which was centrifuged at 1,000 g for 5 min at 4° C. to isolate serum samples from whole blood, which were collected, aliquoted, and stored at −80° C. for identification of NAPQI, and liver function tests (LFTs).

### LC-MS/MS Method for NAPQI Identification and Characterization of Novel Benzoquinoimine

N-acyl-p-benzoquinone imine (NAPQI) was extracted from the serum by adding 3 volumes of ethyl-acetate to 1 volume of serum and storing them on ice for 30 min, followed by centrifugation at 3000 g for 30 min. Afterward, the supernatant was transferred into mass spectrometry vials and dried under N₂ gas. The serum was then washed with another 3 volumes of ethyl-acetate, and the supernatant was added back to the same mass spectrometry vial, followed by evaporation under a stream of N₂ gas. The sample was re-suspended with 50 μl of 1:1 MeOH:H₂O for LC-MS/MS experimentation. The mass spectrophotometer was operated in multiple reaction monitoring (MRM) mode using positive ion electrospray. NAPQI was detected by monitoring the m/z transition 150.3→108.1. Pure NAPQI analytical standard (10 ng/mL) obtained from Cayman Chemical (Ann Arbor, MI) was used to characterize the full fragmentation pattern of NAPQI and determine the likely fragments.

LC-MS/MS Methods for Detection of D₁₀-SRP-001 and AM404 in In Vivo Pain Models (Changed Order so that Both LC-MS/MS Methods are Together)

Deuterated compound D₁₀-SRP-001 was synthesized by Olon Ricerca Bioscience (Concord, OH), and purity was determined to be >99.9% by HPLC. This was used as pure analytical standard for detection of SRP-001 using an LC-MS/MS method. Similarly, pure analytical standard of AM404 (10 ng/mL) obtained from Cayman Chemical (Ann Arbor, MI) was used to characterize the full fragmentation pattern of AM404. Vehicle (0.9% saline), ApAP or SRP-001 was injected ip at 32 mg/kg into Sprague-Dawley rats. Thirty min post-injection, the animals were sacrificed (same as in the CFA/von Frey model), their brains harvested, and the midbrain periaqueductal gray (PAG) region excised. SRP-001 or AM404 were extracted from the PAG using a liquid-liquid extraction method using 1:1 MeOH:H₂O for LC-MS/MS and were loaded onto a liquid chromatography-tandem mass spectrophotometer for analysis. The mass spectrophotometer was operated in multiple reaction monitoring (MRM) mode using positive ion electrospray. D₁₀-SRP-001 was detected by monitoring the m/z transition 416→186. AM404 was detected by monitoring the m/z transition 396→287.

### Liver Function Tests (LFTs)

Liver transaminases—Alanine Aminotransferase (ALT), and Aspartate Aminotransferase (AST), were measured using fresh serum samples by commercially available ELISA kits from Sigma-Aldrich and Abcam, according to the manufacturer's suggested protocols. LFTs were done on fresh serum samples, and not on thawed frozen serum samples to avoid denaturing LFT transaminases due to freeze/thaw of frozen serum.

### Histology and Immunohistochemistry for Nitrotyrosine, Hepatic Tight Junctions, and TUNEL Apoptosis Assays

Male CD1 mice fasted overnight with only access to water ad libitum were dosed with either ApAP, or SRP-001 or vehicle (0.9% saline) (n=5 for each treatment group) via PO dosing at concentrations of 600 mg/kg (for both ApAP and SRP-001) with an injection volume of 10 mL/kg body weight. We selected this dosage based on its well-documented characterization in previous research as an effective measure for evaluating ApAP-induced hepatotoxicity in preclinical rodent models. Employing a consistent lethal dose for both compounds streamline the experimental design and facilitates data interpretation. This approach aligns with FDA guidelines on Bioequivalence and Bioavailability studies for estimating the maximum safe starting dose in humans, a strategy further substantiated by others. At 12 h post-injection, mice were deeply anesthetized under 5% isoflurane for more than 5 min and formalin-fixed via transcardial perfusion with 10% Neutral Buffered Formalin (NBF) after exsanguination using 0.9% NaCl. Liver tissues were extracted and stored in NBF for 24 h post fixation, after which they were then transferred to 80% EtOH for storage prior to paraffin embedding, sectioning, and immunological staining. FFPE blocks were sectioned into 5 μm thick slices on regular frost-free plus slides and dried overnight on a slide warmer. After deparaffination, sections were stained with validated antibodies for nitrotyrosine and ZO-1 (tight junctions). For detection of apoptotic nuclei by TUNEL staining, we used Promega's DeadEnd fluorometric TUNEL assay, following the manufacturer's suggested protocol. Nitrotyrosine labeling was detected with 3′3′-Diaminobenzidine (DAB) staining, and nitrotyrosine labeled hepatic sections were imaged at 100× magnification in brightfield using a Nikon Eclipse TS100 microscope with NIS-Elements BR 3.0 software (NIKON Inc, Melville, NY, USA). Slides with ZO-1 staining were imaged in z-stacks obtained at 20× magnification using Olympus FV-1200 confocal microscope with Fluoview software FV10-ASW Version 04.02.02.09 (Olympus Corp Center Valley, PA, USA). Quantification of ZO-1 staining hepatic tight junctions was carried out by unbiassed image analysis calculating area sum of pixel² of the green channel staining ZO-lusing CellSens software (Olympus Corp Center Valley, PA, USA). TUNEL-positive apoptotic nuclei were counted in 15 random fields from hepatic sections obtained from mice treated with each compound—vehicle, ApAP, or SRP-001—using ImageJ software 1.48 (National Institutes of Health).

### Animal Experiments

All animal protocols and procedures were completed under the pre-approved provisions of the Institutional Animal Care and Use Committee (IACUC) of Louisiana State University Health Sciences Center (LSUHSC), New Orleans. The experiments were performed under the approval by the Institutional Animal Care and Use Committee, LSU Health New Orleans, IACUC protocol #3739. In vivo antinociception was tested in two different strains of mice: CD1 and C₅₇BL/6 mice and Sprague-Dawley rats. All laboratory rodents were purchased from a commercial vendor (Charles River); animals were acclimated to the LSUHSC New Orleans Neuroscience Center of Excellence vivarium for at least seven days before experimental protocols began. All animals were kept in a 12 h day-night cycle with food and water available ad libitum.

### Equimolar Dose-Response Toxicity Analysis

To accurately compare the toxicity profiles of ApAP and SRP-001, we conducted an equimolar dose-response toxicity analysis. CD-1 male mice obtained from Charles River Laboratories underwent a one-week acclimation period. They were housed under a 12-hour day/night cycle, with unrestricted access to food and water. Before experimentation, mice were fasted for 15 hours. Subsequently, they were administered either a vehicle (placebo), ApAP, or SRP-001 in equimolar doses of 992, 1,984, 3,969, or 5,954 mM/kg, respectively, via oral gavage. This corresponds to an increasing concentration of 150, 300, 600, and 900 mg/kg for ApAP and 402, 804, 1,609, and 2,414 mg/kg for SRP-001. Each treatment group consisted of 10 mice. Post-dosing, the mice were monitored at 6-hour intervals over 72 hours for signs of becoming moribund. An investigator blinded to the treatment groups conducted the moribund state assessment.

In Vivo Analgesia Models and Equimolar (μmol/kg) ED₅₀ Calculation

We explored the antinociceptive properties of SRP-001 using the oral nanosuspension of SRP-001. The oral nanosuspension formulation containing 100 mg/mL SRP-001 in preserved aqueous 1% hydroxypropyl cellulose (1% HPC) (Lot #LPI-2021028) was compared to Acetaminophen (ApAP) 100 mg/mL and a vehicle control (aqueous 1% HPC) also given as suspension, in three different in vivo pain animal models—namely, Complete Freund's Adjuvant (CFA) induced inflammatory, tail flick somatic, and abdominal writhing visceral assays, along with electronic von Frey (eVF) and Hargreaves determination of mechanical and thermal (noxious heat) sensitivity, respectively.

The investigators were blinded till the completion of all the experiments. To ensure objectivity, investigators were blinded for the acetic acid and tail flick experiments. In the CFA/von Frey and Hargreaves assays, one investigator did the drug pre-treatments and another investigator at another location who was not aware of the treatments conducted the behavioral testing. Moreover, these studies used the eVF device that electronically registers the grams of force necessary to elicit paw withdrawal, eliminating bias that confounded older versions of this test in which an investigator used a series of individual von Frey microfilaments to determine paw withdrawal thresholds. Six separate experimenters were involved in the data acquisition for behavioral testing and another separate investigator performed all the statistical analyses using GraphPad Prism Version 9.1.2. Statistical significance was determined by p<0.05; one-way ANOVA followed by Sidak's multiple comparisons post hoc test. Mixed-gender experiments were powered to test for gender effects, and different ages of rodents were also included for the experiments—young (2 months) and aged (20 months) to tease out whether there are any effects of age-related changes in nociceptive sensitivity under naïve, and inflammatory pre-clinical rodent pain models. For the aged rats and mice, we could only obtain male Sprague-Dawley rats and male C₅₇BL/6 mice of 20 months old from all commercial animal vendors and from the National Institute of Aging (NIA). So, only these rodents were used for the older animal experimental cohort. Old female rats and mice were not available at the time when these experiments were conducted. For ED₅₀ calculation, dose response curves were calculated with regression analysis and log transformation using GraphPad Prism 9.1.2.

CFA/Von Frey with Electronic Detection (eVF)

First, we used male Sprague-Dawley rats and the CFA inflammatory pain assay with electronic von Frey detection to assess antinociceptive/anti-hyperalgesic efficacy of SRP-001 compared to ApAP and vehicle control. In this model of mechanonociception, one hind paw at a time is stimulated with an electronic von Frey (eVF) filament (noxious source) until the animal retracts the paw from the mechanical stimulus. von Frey tests were conducted in a dedicated room at the LSUHSC New Orleans Neuroscience Center of Excellence vivarium. Rats were acclimated to their environment for 2 days for 30 min/day prior to testing. To obtain eVF pressure recordings, each animal was placed in an individual plastic observation compartment on a perforated metallic grid platform, which provided access to the plantar surface of the hind paws. After acclimation to the environment for 30 min, mechanical hypersensitivity was assessed by stimulating the mid-plantar area of each hind paw with a rigid tip von Frey filament attached to the eVF meter (Ugo Basile 38450) until animals withdrew the paw from the filament. The withdrawal threshold was defined as the average force/pressure (g) required for the rat to withdraw the stimulated paw. A brisk withdrawal of the paw (often followed by a sustained retraction and/or licking) was considered a positive response, but paw withdrawals due to locomotion or weight shifting were not counted. The von Frey studies were conducted with an eVF device that electronically registers the grams of force necessary to elicit paw withdrawal in order to eliminate bias that confounded previous older versions of this test. Baseline withdrawal thresholds of both the right and left paws were recorded, and animals were assigned to different treatment groups so that each group had approximately equal withdrawal threshold averages in both paws. After baseline testing, treated animals received subcutaneous plantar injection (150 μl) of 50% CFA diluted and freshly mixed in sterile (0.9%) saline into the left hind paw unilaterally, while control animals received a plantar injection (150 μl) of 0.9% NaCl into their left hind paw. CFA induces inflammation, resulting in a left hind paw that is hypersensitive to mechanical stimulus, while the right hind paw serves as a within-subject baseline for each animal. In the experimental setup, the left hind paw of rats was administered either CFA or saline, while the right hind paw functioned as an uninjected control in CFA-treated animals for each pharmacological agent, be it ApAP or SRP-001. On the day of testing, each animal received the drugs—ApAP or SRP-001 via PO administration based on their assigned treatment groups. Doses at concentrations of 32 mg/kg and 100 mg/kg were tested in a cumulative dose-response manner and given at an interval of 60 min, and paw withdrawal threshold readings were measured using eVF as described above.

Additionally, we determined ED₅₀ values on an equimolar (μmol/kg) basis for both ApAP and SRP-001 across all in vivo antinociception assays (FIG. 2j, and FIG. 10).

Drawing upon our previously published work, wherein dose-response curves for ApAP and its novel analogs were established across various dosages—10, 32, 100, and 320 mg/kg, we selected median doses of 32 and 100 mg/kg for the current study. Our experimental design adhered to the principles of the 3Rs—Replacement, Reduction, and Refinement—as outlined by Russel and Burch, aiming to minimize ‘unnecessary duplication’ and reduce the number of animals needed for robust, replicable data of 3Rs. Additionally, we determined ED₅₀ values on equimolar (μmol/kg) basis for both ApAP and SRP-001 across all in vivo antinociception assays (FIG. 2J, and FIG. 10).

Young Male Rats. In this cohort, n=40 male Sprague-Dawley rats (2 months) were used, and two different doses of SRP-001 oral nanosuspension and ApAP (32 and 100 mg/kg) were compared to a vehicle control.

Young Female Rats. In this cohort, n=40 female Sprague-Dawley rats (2 months) were used, and two different doses of SRP-001 oral nanosuspension and ApAP—32 and 100 mg/kg were compared to a vehicle control.

Aged Male Rats. In this cohort, n=20 male Sprague-Dawley rats (20 months) and two different doses of SRP-001 oral nanosuspension and ApAP—32 and 100 mg/kg were compared to a vehicle control.

### Tail Flick.

We used cold stimulation tail flick somatic pain assay testing 2 different doses of ApAP or SRP-001 at 32 m/kg or 100 mg/kg to measure increased tail withdrawal time (latency) in CD1 (young male and female) or C57BL/6 (aged male) mice to cold stimulation of the tail tip. Half of the tail was immersed in ice-cold water maintained at 4° C. For the tail-flick assays, we used n=70 aged male mice, n=90 young male mice, and n=120 young female mice. Based on the availability of mice at the time of the experiments, different numbers of animals were assigned in different cohorts of experimental animals. Tail flick assay experiments were conducted on an open bench in a dedicated room at the LSUHSC New Orleans Neuroscience Center of Excellence vivarium. Mice were allowed to acclimate to the laboratory environment for 1 h prior to testing. To restrain the mice for the test, disposable plastic 50 mL screw-capped conical centrifuge tubes were cut at the tip to create a 0.5 cm opening to allow the mice to breathe freely. Another 0.5 cm opening was cut into the cap to allow access of the tail to the water bath. A 500 mL glass beaker was filled with 450 mL of ice-cold distilled water maintained at 4° C. with the addition of ice and determined with a glass thermometer. Mice were held over the opening of the water bath, and their tails submerged approximately halfway into the water. The nociceptive threshold was taken as the latency until the mice flicked their tail tip or removed the tail. The time from immersion to the attempted tail tip removal was measured to 1/10^{th} of a sec with a digital laboratory timer. To minimize damage to the tail, a 60-sec cut-off was utilized. After baseline measurements, mice were dosed with ApAP or SRP-001 via PO administration based on their assigned treatment groups. Injections were at doses of 32 mg/kg and 100 mg/kg. 30 min post-drug administration tail-flick/withdrawal latency was measured.

### Abdominal Writhing Assay

In this model of visceral pain, abdominal contraction (writhing) in which contraction of the abdominal muscles and stretching of the hind limbs is induced in mice in response to an ip injection of 0.4% acetic acid at a dose of 10 mL/kg 25 min after drug administration is assessed. Specifically, the number of writhes is counted for 10 min beginning 5 min after acetic acid injection. All animals were fasted overnight (15 h) prior to testing, and the compounds were administered via PO administration to animals belonging to the treatment groups—ApAP or SRP-001— and tested at doses of 32 mg/kg and 100 mg/kg, respectively. CD1 (young male and female) or C57BL/6 (aged male) mice were used. For the abdominal writhing assays, we used n=35 aged male mice, n=70 young male mice, and n=70 young female mice.

### CFA-Induced Inflammatory Pain/Hargreaves Thermal Sensitivity Antinociception Assay

First, we used male Sprague-Dawley rats and the CFA inflammatory pain assay with eVF detection to assess antinociception/anti-hyperalgesic efficacy of SRP-001 compared to ApAP and vehicle control. In these same cohorts of animals, we simultaneously measured their thermal nociception to hyperalgesia with the Hargreaves test using the plantar test apparatus (Ugo Basile 37570). In this experimental setup, the rodents are placed in plastic cages on top of a glass surface, and their hind paws are subjected to an infrared heat stimulus. One hind paw at a time is stimulated with the light source (noxious stimuli) until the animal retracts the paw from the glass surface because of the stimulus. Baseline withdrawal thresholds (latency) of both the right and left paws were recorded, and animals were assigned to different treatment groups so that each group had approximately equal withdrawal threshold averages in both paws. After baseline testing, treated animals received subcutaneous plantar injection (150 μl) of 50% CFA into the left hind paw, while control animals received a plantar injection (150 μl) of 0.9% NaCl into their left hind paw. CFA induces inflammation, resulting in a left hind paw that is hypersensitive to the thermal stimulus, while the right hind paw serves as a within-subject baseline for each animal. To obtain Hargreaves withdrawal latency recordings, each animal was placed in an individual plastic observation compartment on a glass platform, which provided access to the plantar surface of the hind paws from underneath. After acclimation to the environment for 30 min, thermal hypersensitivity was assessed by stimulating the mid-plantar area of each hind paw with a bright light source attached to the meter (Ugo Basile 37570) until animals withdrew the paw from the surface. The withdrawal threshold was defined as the average time (s) required for the rat to withdraw the stimulated paw. A brisk withdrawal of the paw (often followed by a sustained retraction and/or licking) was considered a positive response, but paw withdrawals due to locomotion or weight shifting were not counted. The Hargreaves studies were conducted with the plantar stimulation device that automatically registers the time (s) necessary to elicit paw withdrawal in order to eliminate bias in determining paw withdrawal thresholds. A cutoff of 20 s is pre-programmed so that the light source shuts off at that maximal period so as to not induce any burns on the plantar surface of the rodents. The Hargreaves test permits measurement of ipsilateral and contralateral heat thresholds, allowing each animal to serve as its own internal control in unilateral pain models. In addition, the Hargreaves test enables quantification of heat thresholds in unrestrained animals, reducing the likelihood of stress-induced responses.

Young Male Rats. In this cohort, n=40 male Sprague-Dawley rats (2 months) were used, and two different doses of SRP-001 oral nanosuspension and ApAP—32 and 100 mg/kg were compared to a vehicle control.

Young Female Rats. In this cohort, n=40 female Sprague-Dawley rats (2 months) were used, and two different doses of SRP-001 oral nanosuspension and ApAP—32 and 100 mg/kg were compared to a vehicle control.

Aged Male Rats. In this cohort, n=20 male Sprague-Dawley rats (20 months) were used, and two different doses of SRP-001 oral nanosuspension and ApAP—32 and 100 mg/kg were compared to a vehicle control.

### In Vivo Antipyresis Assays

Antipyresis experiments were carried out with n=60 CD-1 male mice weighing between 45-50 g. All mice were kept in a 12-h day/12-h night cycle with free access to food and water ad libitum. After habituating and acclimating the mice for a week, each mouse was briefly anesthetized for 1 min with 1% isoflurane and implanted subcutaneously a transmitter probe that records core body temperature—Implantable Programmable Temperature Transponder (IPTT-300) by Bio Medic Data Systems (BMDS), DE, USA. The mice were allowed to recover for at least one week from the surgery before any recordings were made and observed in their home cages prior to pyrogen challenge for inducing fever. Temperatures were recorded using the BMDS DAS-8027-IUS data reading system.

### Pyrogen (LPS) Injections

All mice were fasted overnight (15 h) with free access to water ad libitum prior to the pyrogen challenge. For the induction of fever, LPS from E. coli (100 μg/kg, 0111:B4) (MilliporeSigma, Billerica, MA, USA) was used. Mice received either LPS in 0.9% saline (sterile) (vehicle) or just 0.9% saline as an intraperitoneal (ip) injection. Mice were returned back to their home cages post-injection and observed, and core body temperatures were recorded at the 4-h mark post-injection, and with the development of fever (˜at least 1 degree change from basal core body temperature), febrile mice were selected and grouped in different groups for drug administration, and temperatures were recorded 2-h post drug administration to determine the antipyretic effects of ApAP or SRP-001. All mice that did not show a significant change in body temperature or appeared sick post-pyrogen challenge (n=5) were excluded from subsequent drug administrations. No mice were excluded from the treatment groups that were dosed with either ApAP or SRP-001 (75 mg/kg).

Drug injection/concentration: All drugs and vehicle (10% DMSO: 10% labrasol:80% PEG400) were administered by oral gavage, PO dosing. Each dosing volume was ˜0.3-0.4 mL per mouse. ApAP, SRP-001 and vehicle were all prepared as suspensions. The dose for ApAP and the novel compound, SRP-001, used for this trial was 75 mg/kg. We chose this dose to record antipyretic effect in a low dose that doesn't cause hypothermia in mice. Febrile mice were grouped into different control and experimental groups as follows:
n=10 for Normal Control (Baseline)
n=20 for Positive Control (APAP) and
n=20 for SRP-001
   Normal Control (Baseline)—0.9% Saline (ip) and 0.9% saline (po)
   Positive Control (ApAP)—LPS (100 μg/kg) (ip) and APAP (75 mg/kg) (po)
   SRP-001—LPS (100 μg/kg) (ip) and SRP-001 (75 mg/kg) (po)

### Baker Yeast-Induced Hyperthermia

(15% yeast, 0.1 mL/kg) (MilliporeSigma, Billerica, MA, USA) was used to induce fever. All animals used in this assay were male CD1 mice, which were fasted overnight (15 h) before the start of the assay. Mice received either yeast in 0.9% saline (sterile) (vehicle) or just 0.9% saline as an ip injection. Mice were returned back to their home cages post-injection and observed, and core body temperatures recorded at the 4-h mark post-injection, and with the development of fever (˜at least 1 degree change from basal core body temperature), febrile mice were selected and grouped in different groups for drug administration, and temperatures were recorded 2 h post drug administration to determine the antipyretic effects of ApAP or SRP-001. All mice that didn't show a significant change in body temperature or appeared sick post-pyrogen challenge were excluded (n=5) from subsequent drug administrations. No mice were excluded from the treatment groups that were dosed with either ApAP or SRP-001 (75 mg/kg).

Drug injection/concentration: All drugs and vehicle (10% DMSO: 10% labrasol:80% PEG400) were administered by oral gavage, PO administration. Each dosing volume was ˜0.3-0.4 mL per mouse. ApAP, SRP-001 and vehicle were all prepared as suspensions. The dose for ApAP and the novel compound, SRP-001, used for this trial was 75 mg/kg. We chose this dose to record antipyretic effect in a low dose that doesn't cause hypothermia in mice. Febrile mice were grouped into different control and experimental groups as follows:
n=10 for Normal Control (Baseline)
n=10 for Positive Control (APAP) and
n=10 for SRP-001
   Normal Control (Baseline)—0.9% Saline (ip) and 0.9% saline (po)
   Positive Control (ApAP)—yeast (15%) (ip) and APAP (75 mg/kg) (po)
   SRP-001—yeast (15%) (ip) and SRP-001 (75 mg/kg) (po)
      Cardiotoxicity Assay (hERG) and Cardiovascular Effect in Conscious Telemetered Beagle Dogs (GLP Study)

The effects of SRP-001 on hERG current amplitude at physiologic temperatures were evaluated over a nominal concentration range of 0.1 μM to 100M. A vehicle group (10% DMSO) was included in the study for comparison, and dofetilide (10 nM) was used as a reference substance. The vehicle and positive controls indicated appropriate sensitivity of the test systems. SRP-001 was a weak blocker of hERG current with the highest concentration tested (100 μM) blocking by 21.4±3.9%. Next, the potential acute effects of oral SRP-001 on cardiovascular function in conscious telemetered beagle dogs was done using a Latin Square design; 4 male naïve beagle dogs each received vehicle (10% DMSO: 10% labrasol: 80% PEG400) and three doses (30, 90, and 150 mg/kg) of SRP-001 nanosuspension by oral gavage. The animals were observed for clinical signs pre-dose, immediate post-dose, and approximately 24 h post-dose administration. A single dose was administered on the day of dosing, and a washout period of seven days was allowed between doses. One-min means of hemodynamic parameters, as well as ECG parameters and body temperature, were measured for a period of at least 24 h. One-min tracings of the ECGs were obtained at 15 min prior to dosing and at 30 min, 1, 2, 4, 8, 12, and 24 h post-dose. Qualitative evaluation of the ECGs was done by a board-certified veterinary cardiologist.

### Pulmonary Function Evaluation in Conscious Male Sprague-Dawley Rats (GLP Study)

The potential effects of orally administered SRP-001 on respiratory rate, tidal volume, and minute volume in conscious male Sprague-Dawley rats was studied in 24 male rats. The rodents were trained for two days in the head-out plethysmograph chamber for 15-17 min each prior to the experiment. On the day of dosing, each animal was weighed and placed in the plethysmograph chamber, and baseline respiratory parameters were obtained for 5 min following an approximately 5-min stabilization period. The rats were then removed from the chamber and dosed by oral gavage. Three groups of six male Sprague-Dawley rats each were orally administered SRP-001 at a single dose of 220, 440, or 660 mg/kg at a dose volume of 10 mL/kg. An additional group of six rats was administered the vehicle (10% Labrasol: 10% DMSO: 80% PEG400) at 10 mL/kg. Following dosing, each animal was returned to its designated plethysmograph chamber, and the respiratory parameters were recorded at 30 min (±5 min), 2, 4, and 6 h (±15 min). Animals were allowed to stabilize in the plethysmograph chamber for at least 5 min before each reading was taken. The following parameters were acquired, recorded, and analyzed using Ponemah Physiology Platform (Ponemah v.5.20 Pulmonary); Respiratory rate, Tidal Volume, and Minute Volume, Values for the test article treated group were compared to the vehicle control values using a two-way repeated measures ANOVA followed by a Bonferroni Multiple Comparison Test (SigmaStat, v. 4.0). Differences with p values≤0.05 were considered statistically significant.

### Neuropharmacological Profile in Sprague-Dawley Rats (GLP Study)

Any potential neuropharmacological effects of orally administered SRP-001 in rats were assessed in three groups of eight male Sprague-Dawley rats. Each was orally administered SRP-001 at a single dose of 220, 440, or 660 mg/kg. Vehicle (10% labrasol: 10% DMSO: 80% PEG400) was administered to an additional group of eight rats. The dose volume was 10 mL/kg. The rats were observed for neuropharmacological or other clinical signs using a modified Irwin/Functional Observational Battery test at 30 min and 1, 2, 4, and 24 h following treatment. All observations were made at ±5 min for the observations through 1 h post-dose and ±15 min for the later observations. Body temperature was measured 1 h following dose administration. No apparent neuropharmacological or clinical signs were observed through 24 h post-dose in any rats receiving either vehicle at 10 mL/kg or SRP-001 at 220, 440, or 660 mg/kg. Body temperature was not affected by the oral administration of SRP-001 at 220 or 440 mg/kg at 1 h post-dose when compared to the vehicle control group. However, the body temperature was significantly (p≤0.05) lower in the high-dose group (660 mg/kg) compared to the vehicle group.

### Genotoxicity

Results from both the Ames and micronucleus assays indicate that SRP-001 is negative for genotoxicity induction (FIG. 26, Table 1). However, the chromosomal aberration assay results indicated that SRP-001 was positive for the induction of chromosomal aberrations in vitro. To further evaluate the potential genotoxicity of SRP-001 and determine the validity of the in vitro findings, a second in vivo assay, a GLP-compliant Comet assay, was conducted in accordance with ICH S2(R1) Guidance on Genotoxicity Testing and Data Interpretation for Pharmaceuticals Intended for Human Use. SRP-001 did not cause DNA damage in the liver or stomach of male rats administered up to 2,000 mg/kg and was concluded to be negative in the in vivo Comet assay. Based on the weight of the evidence, it is concluded that SRP-001 is negative for genotoxicity. This is based on the following:
1) A negative Ames mutagenesis demonstrates SRP-001 non-mutagenic. SRP-001 was tested to evaluate its mutagenic potential by measuring its ability to induce reverse mutations at selected loci of several strains of Salmonella typhimurium (S. typhimurium) and at the tryptophan locus of Escherichia coli (E. coli) strain WP2 uvrA in the presence and absence of an exogenous metabolic activation system (Bioreliance, Rockville, MD USA). Dimethyl sulfoxide (10% DMSO) was used as the vehicle. The dose levels tested in the preliminary toxicity assay were 6.67, 10.0, 33.3, 66.7, 100, 333, 667, 1000, 3333, and 5000 μg per plate. No precipitate was observed. Toxicity as a reduction in the revertant count was observed at 5000 μg per plate with tester strains TA1537 and WP2 uvrA in the absence of S9 activation. Based on these results, the maximum dose tested in the mutagenicity assay was 5000 μg per plate. The dose levels tested in the mutagenicity assay were 100, 333, 667, 1000, 3333, and 5000 μg per plate. Neither precipitate nor toxicity was observed. No positive mutagenic responses were observed with any of the tester strains in either the presence or absence of S9 activation. Collectively, these results indicate SRP-001 is negative for the ability to induce reverse mutations at selected loci of several strains of S. typhimurium and at the tryptophan locus of E. coli strain WP2 uvrA in the presence and absence of an exogenous metabolic activation system.
2) In Vitro Mammalian Chromosomal Aberration Assay in Chinese Hamster Ovary Cells. In the preliminary toxicity assay, the doses tested ranged from 0.0405 to 405 μg/mL (1 mM), which was the limit dose for this assay (Bioreliance, Rockville, MD USA). Cytotoxicity (≥45% reduction in cell growth index relative to the vehicle control) was not observed at any dose in the non-activated and S9-activated 4-h exposure groups. Cytotoxicity was observed at doses ≥122 μg/mL in the non-activated 20-h exposure group. Based on these results, the doses chosen for the chromosome aberration assay ranged from 75 to 405 μg/mL for the non-activated and S9-activated 4-h exposure groups and from 5 to 125 μg/mL for the non-activated 20-h exposure group.

In the chromosome aberration assay, cytotoxicity (≥45% reduction in cell growth index relative to the vehicle control) was not observed at any dose in the S9-activated 4-h exposure group. Cytotoxicity was observed at 405 μg/mL in the non-activated 4-h exposure group and at doses ≥70 μg/mL in the non-activated 20-h exposure group. The doses selected for evaluation of chromosome aberrations were 150, 300, and 405 μg/mL for the non-activated and S9-activated 4-h exposure groups; and 10, 40, and 80 μg/mL for the non-activated 20-h exposure group. In the non-activated 4-h exposure group, statistically significant and dose-dependent increases in structural aberrations (4.3% and 12.7%) were observed at doses 300 and 405 μg/mL, respectively (p<0.01; Fisher's Exact test and p 0.05; Cochran-Armitage test). The induction of structural aberrations was outside the 95% control limit of the historical negative control data of 0.00% to 2.79%. In the S9-activated 4-h exposure group, statistically significant and dose-dependent increases in structural aberrations (3.7% and 3.3%) were observed at doses 300 and 405 μg/mL, respectively (p<0.05; Fisher's Exact and Cochran-Armitage tests). However, the induction of structural aberrations was within the 95% control limit of the historical negative control data of 0.00% to 4.28%. In the non-activated 20-h exposure group, a statistically significant and dose-dependent increase in structural aberrations (3.7%) was observed at 80 μg/mL (p<0.01; Fisher's Exact test and p<0.05; Cochran-Armitage test). The induction of structural aberrations was outside the 95% control limit of the historical negative control data of 0.00% to 2.68%.

Neither statistically significant nor dose-dependent increases in numerical (polyploid or endoreduplicated cells) aberrations were observed at any dose in treatment groups with or without S9 (p>0.05; Fisher's Exact and Cochran-Armitage tests). The induction of numerical aberrations was within the 95% control limit of the historical negative control data. These results indicate SRP-001 was positive for the induction of structural chromosomal aberrations and negative for the induction of numerical chromosomal aberrations in the presence and absence of the exogenous metabolic activation system.
3) Clastogenicity and rat micronucleus; in vivo study: SRP-001 is deemed negative (non-clastogenic).
Male rats were dosed at 500, 1,000, and 2,000 mg/kg/d once per day on two consecutive days at 10 mL/kg via oral gavage using 10% labrasol, 10% DMSO, and 80% PEG400 as the vehicle (Bioreliance, Rockville, ID USA). Approximately 48 h after the second dose administration, peripheral blood was collected for flow cytometric analysis of micronuclei. There was no significant increase in the incidence of micronuclei in the test article-dosed animals compared to the concurrent vehicle control. The vehicle control value was compatible with the historical range of % micronucleated reticulocytes (MnRETs). There was a statistically significant increase in MnRETs in the positive control compared to the concurrent vehicle control. Statistically significant decreases in % RETs were observed in the treatment groups, indicating the compound induced cytotoxicity. Hence, SRP-001 was evaluated as negative (non-clastogenic) under the conditions of this study.
4) Second in vivo study, Comet assay GI tract: SRP-001 is non-DNA damaging.
Male Sprague-Dawley rats were orally administered SRP-001 at 500, 1000, or 2000 mg/kg/dose for two consecutive days at a dose volume of 10 mL/kg/dose (Bioreliance, Rockville, MD USA). The vehicle control was 10% Labrasol, 10% DMSO, and 80% PEG400. The positive control for the Comet assay, ethyl methanesulfonate (EMS), was orally administered at 200 mg/kg/dose for two consecutive days at a dose volume of 10 mL/kg/dose. This positive control is an alkylating agent; the negative result for SRP-001 indicates that it did not alkylate DNA to a measurable extent under the conditions of this assay. Animals were euthanized 3-4 h after the last dose administration. The liver and glandular stomach were collected and processed for comet evaluation.

Liver. No statistically significant increases in % tail DNA were observed in SRP-001-treated groups. The group mean % tail DNA for the vehicle control was within the 95% control limit of the study historical data range, with one animal (#160) exceeding the individual animal 95% control limit but within the minimum/maximum range. Additionally, three animals (#173, 174, and 175) in the mid-dose (1000 mg/kg/dose) group likewise exceeded the individual animal 95% control limit but were within the minimum/maximum range. The group mean % tail DNA for the positive control was significantly increased when compared to the concurrent group mean % tail DNA for the vehicle control and was compatible with the positive control database for both the study and individual animals.

Glandular Stomach. No statistically significant increases in % tail DNA were observed in the SRP-001-treated groups. The group mean % tail DNA for the vehicle control was within the 95% control limit of the study historical data range, with one animal (#158) exceeding both the individual animal 95% control limit and minimum/maximum ranges. One animal (#171) in the mid-dose (1000 mg/kg/dose) group and one animal (#180) in the high-dose (2000 mg/kg/dose) group exceeded just the 95% control limit range but were within the minimum/maximum range. One animal (#170) in the mid-dose (1000 mg/kg/dose) group and one animal (#179) in the high-dose (2000 mg/kg/dose) group exceeded both the individual animal 95% control limit and minimum/maximum ranges. The group mean % tail DNA for the positive control was significantly increased when compared to the concurrent group mean % tail DNA for the vehicle control and was compatible with the positive control database for both the study and individual animals. All valid assay criteria were met, and SRP-001 was determined to be negative (non-DNA damaging) in the Comet assay.

### Non-GLP and GLP Toxicology

1) Dose-Range-Finding Oral Toxicity Study with SRP-001 in Sprague-Dawley Rats (non-GLP) As part of a two-phase study, the maximum tolerated dose (MTD) of SRP-001 when administered via gavage once to Sprague-Dawley Rats using an ascending/descending dose design (Calvert Labs/Altasciences, Scott Township, PA USA). A total of 12 male Sprague-Dawley rats (3/dose group), approximately 8-9 weeks old and weighing 245-307 g, were administered single doses of 5000, 1000, 1500, or 2000 mg/kg SRP-001. Mortality/morbidity was performed twice daily and once prior to scheduled sacrifice. Clinical observations were evaluated prior to each dose administration and approximately 1-3 h post-dose, once daily on non-dosing days and additionally as needed. Body weights were recorded for animals in Groups 1-4 prior to randomization/selection, each dose administration, and scheduled sacrifice on day 3. Food consumption was recorded on day 3. Nearly all the animals appeared normal following dosing, and no test article-related effects on body weight or food consumption were observed. All animals appeared unremarkable at gross necropsy. Based on the results of this study, doses for the second phase were selected.

2) A Single Oral Dose Toxicity and Pharmacokinetic Study of SRP-001 in Beagle Dogs (non-GLP) SRP-001 was prepared into capsules for oral administration. Three experimentally non-naïve Beagle dogs, at least 7 months old and weighing 7.7 to 10 kg prior to treatment initiation, were administered a single dose of 300 mg/kg SRP-001 (Calvert Labs/Altasciences, Scott Township, PA USA). On the day of dosing, animals were observed prior to dose administration, approximately 1-3 h post-dose, and once on day 2. Body weights were recorded for all animals prior to dose administration for the preparation of the capsules. Blood for bioanalytical evaluation was collected at selected timepoints on day 1. Following the completion of the study, surviving animals were not euthanized. They were returned to the Calvert colony for an appropriate washout period before possible use in a subsequent study. The dose was well tolerated, and all animals appeared normal following dosing. Plasma samples were collected at seven different time points post-dose. Bioanalytical analysis revealed poor bioavailability was achieved following dosing. Capsule dosing, while well tolerated in dogs at 300 mg/kg, was not considered suitable for future studies.

3) A Dose-Range-Finding and 7-day Repeat-Dose Oral Toxicity Study of SRP-001 in Beagle dogs (Non-GLP) Two beagle dogs (1/sex) were administered 30, 100, 250, or 500 mg/kg SRP-001. Animals were observed for clinical signs of toxicity or effect prior to dose administration and approximately 1-3 h post-dose, additionally as needed, and daily on non-dosing days (Calvert Labs/Altasciences, Scott Township, PA USA). After a period of at least 2 d (minimum of 44 h) following the previous dose of test article, additional dose levels of SRP-001, as indicated above, were administered by oral gavage to the same 2 dogs (1 male and 1 female). Each dose was based on the dog's most recent body weight. Mortality/morbidity was observed twice daily and once prior to scheduled sacrifice. Body weights were recorded prior to randomization/selection, prior to each dose administration, and prior to scheduled sacrifice. At scheduled sacrifice, a gross necropsy was performed, and the tissues were appropriately discarded. Loose feces and emesis were observed following most doses primarily observed within 4 hours post dosing at higher doses, however, no dose-dependent trend was observed. No test article-related effects on body weight or food consumption were observed. At gross necropsy, Phase I animals had some red discoloration in the GI tract, as observed in the duodenum, jejunum, ileum, and rectum. Doses for Phase II of the study were set based on Phase I results. The actual dose amounts selected for Phase II of the study were 0, 30, 100, or 300 mg/kg/d of SRP-001. These doses were administered once daily for seven consecutive days to 1 Beagle dog/sex/dose.

4) A Dose-Range-Finding Oral Toxicity Study with SRP-001 in Sprague-Dawley Rats (Non-GLP) As part of a two-phase study, repeat-dose toxicity and toxicokinetics of SRP-001, when administered via oral gavage once daily to Sprague-Dawley rats for 5 d, was done (Calvert Labs/Altasciences, Scott Township, PA USA). The doses selected for Phase II were based on the results of Phase I. SRP-001 was administered to 20 naïve rats (5/group in Groups 5-8) once daily for 5 days via oral gavage for toxicology evaluation and once to 18 naïve rats (6/group in Groups 9-11) for toxicokinetic evaluation.

Mortality/morbidity was performed twice daily and once prior to sacrifice. Clinical observations were evaluated prior to each dose administration and approximately 1-3 h post-dose for animals in Groups 5-8. Body weights were recorded prior to randomization/selection, prior to dose administration on day 1 for animals in Groups 5-11, and prior to dose administration on day 5 for Groups 5-8. Food consumption was recorded for animals from Groups 5-8 on day 5. Blood for evaluation of hematology, coagulation and clinical chemistry and urine for urinalysis was collected from animals in Groups 5-8 on day 6. All animals in Groups 5-8 were sacrificed on day 5. Selected tissues were harvested at necropsy, selected organs weighed, and selected tissues preserved. Blood for toxicokinetic evaluation was collected from animals in Groups 9-11 at selected timepoints on day 1 to ensure comprehensive data capture. These timepoints include 0 hour (pre-dose) and post-dose intervals of 1, 2, 4, 8, and 24 hours on both Day 1 and Day 28 of the study. This schedule allows for a robust assessment of SRP-001's pharmacokinetic profile, including absorption, distribution, metabolism, and excretion and is in line with current FDA-guidelines for GLP studies.

Clinical signs included soft feces, brown staining of the anogenital area, and a few instances of red staining of the muzzle. These signs were observed in all groups and are likely related to the vehicle (10% Labrasol: 10% DMSO: 80% PEG400) used. Test article-related increases in absolute and relative liver weights and increased cholesterol levels were observed. Dark discoloration of the liver was observed in the majority of control animals and all experimental animals at gross necropsy. No SRP-001-related differences in body weight, food consumption, hematology, or coagulation parameters were observed. AUC and Cmax levels increased in a dose-dependent manner. Half-lives were 6.19 and 6.86 h at mid and high doses, respectively. Tmax ranged from 3 h at the low dose to 4.5 h at mid and high doses. Based on these observations, SRP-001 was considered to be tolerated at 1200 mg/kg when administered once daily for 5 consecutive days.

5) A 28-day Oral Toxicity Study of SRP-001 in Sprague-Dawley Rats with 14-day Recovery (GLP study) We next set out to determine the toxicity and toxicokinetics of SRP-001 when administered via oral gavage once daily to Sprague-Dawley Rats for 28 d (Calvert Labs/Altasciences, Scott Township, PA USA). The study also assessed the reversibility of any toxicity observed with a 14-d recovery period. One hundred thirty-six experimentally naïve Sprague-Dawley rats (68 males and 68 females), 8 weeks old and weighing 167-295 g for males and females at the outset of the study, were assigned to toxicology treatment groups (Groups 1-4) or toxicokinetic groups (Groups 5-7). For toxicology groups, 10 animals/sex/dose were administered 0, 300, 900, or 1500 mg/kg/d SRP-001. An additional 5 animals/sex/dose in Groups 1 and 4 were targeted for a 14-d recovery period and euthanized on day 43. For toxicokinetic groups, 6 animals/sex/dose were administered 300, 900, or 1500 mg/kg/d SRP-001. Mortality/morbidity was performed twice daily and once prior to scheduled sacrifice. Animals in Groups 1-4 were observed prior to each dose administration and 1-3 h post-dose. Animals in Groups 1-4 were also observed once daily on non-dosing days and once prior to scheduled sacrifice. Body weights were recorded prior to randomization/selection, prior to dose administration on day 1, and weekly thereafter. Food consumption was determined weekly for animals in Groups 1-4. Ophthalmology examinations were performed before treatment initiation and during the last week of dosing for animals in Groups 1-4. The functional observational battery was assessed for 5 animals/sex/group in Groups 1-4 once prior to dosing and at 1-2 h following dose administration on day 1. Blood for evaluation of hematology, coagulation, and clinical chemistry and urine for urinalysis was collected from animals in Groups 1-4 on day 29 or day 43. Blood for toxicokinetic evaluation was collected from animals in Groups 5-7 at selected timepoints on days 1 and 28. All surviving animals in Groups 1-4 were sacrificed on day 29 or day 43. Selected tissues were harvested at necropsy, selected organs weighed, and selected tissues evaluated microscopically.

Following dosing, the reversibility of any toxicity observed was assessed with a 14-d recovery period of animals receiving 0 or 1500 mg/kg of SRP-001. SRP-001-related clinical signs included red staining around the forepaw, forelimbs, mouth, muzzle, and nose and brown-staining in the anogenital area. To some extent, these clinical observations appeared to be vehicle-related; however, they were observed at an increased frequency and duration correlating with increasing dose levels. The majority of clinical signs were observed during the first 3 weeks of dosing, and by day 28, the majority of the animals appeared normal. Hematology and serum chemistry analysis on day 29 identified non-adverse SRP-001-related increases in globulin, albumin, and total protein and decreases in aspartate aminotransferase, alkaline phosphatase, and mean corpuscular hemoglobin concentration. These differences remained within or close to the historically normal ranges. Statistically significant differences in total protein, globulin, and mean corpuscular hemoglobin remained present in males after the recovery period, although at a smaller magnitude. SRP-001-related microscopic findings were limited to the liver and included minimal to mild centrilobular hepatocellular hypertrophy in both sexes at ≥300 mg/kg/d. This finding correlated with statistically significant increases in liver weights in both sexes. Hepatocellular hypertrophy was considered to be an adaptive response and non-adverse due to limited severity, lack of histological evidence suggestive of structural damage (such as a concurrent dose-dependent increase in hepatocellular necrosis or inflammation), and lack of dose-dependent and biologically significant increases in clinical chemistry parameters suggestive of hepatobiliary damage.

There were no SRP-001-related effects on body weight, food consumption, functional observation battery, ophthalmological findings, coagulation parameters, or urinalysis parameters. Exposure to SRP-001 following oral gavage dosing at 300, 900, and 1500 mg/kg SRP-001 was dose-dependent, increasing with escalating doses following a single dose. Exposure to SRP-001 following 28 consecutive days of oral gavage doses at 300, 900, and 1500 mg/kg/d SRP-001 was dose-dependent but did not increase as much as seen on day 1. Based on the parameters observed, the no-adverse effect level (NOAEL) of SRP-001, when administered once daily for 28 d to Sprague-Dawley rats, was 1500 mg/kg.

Cardiotoxicity is also absent with SRP-001. This stems from no cardiovascular effects in 28-day GLP studies in both beagle dogs or rats and in a telemetered safety pharmacology study in dogs. It did not result in a relevant signal in the in vitro hERG assay in human embryonic kidney cells at dosages where acetaminophen causes significant changes in these markers is absent with SRP-001. SRP-001 was a weak blocker of hERG current with the highest concentration tested (100 μM) blocking by 21.4±3.9% (FIG. 6). Oral SRP-001 administration at doses of 220, 440, and 660 mg/kg did not induce any significant effects on respiratory rate, tidal volume, or minute volume in conscious male Sprague-Dawley rats. No apparent neuropharmacological or clinical signs were observed through 24 h post-dose in any rats receiving either vehicle at 10 mL/kg or SRP-001 at 220, 440, or 660 mg/kg, except for a lower body temperature in the high dose group (660 mg/kg). Oral administration of SRP-001 at doses of 30, 90, and 150 mg/kg in conscious telemetered beagle dogs did not induce any biologically relevant effects on blood pressure, heart rate, ECG morphology, ECG measurements, or body temperature.

6) A Two-day Oral Dose Toxicity and Pharmacokinetic Study of SRP-001 in Beagle Dogs Non-GLP) SRP-001 was supplied by Olon Ricerca Bioscience as an off-white powder and then prepared as dosing suspensions for oral administration via gavage (Calvert Labs/Altasciences, Scott Township, PA USA). Six experimentally non-naïve male Beagle dogs (3/dose), at least 7 months old and weighing 7.2 to 10.2 kg prior to treatment initiation, were administered 100 or 900 mg/kg/d SRP-001. Animals were dosed three times daily (3-5 h between doses) for 2 d. Mortality/morbidity was performed twice daily. On the day of dosing, animals were observed prior to each dose administration and approximately 1-3 h after each dose. Animals were also observed once on day 3. Body weights were recorded for all animals prior to the first dose administration on days 1 and 3, and food consumption was recorded daily. Blood for bioanalytical evaluation was collected at selected timepoints on day 1. Following the completion of the study, surviving animals were not euthanized and were returned to the Calvert colony.

Nearly all animals had emesis within 1-2 hours following each dose administration. Other clinical signs observed included loose or watery feces and salivation. Nearly all animals appeared normal prior to receiving their next dose. Body weight loss was also observed; however, no animal lost more than 4% of their body weight. Body weight loss correlated with decreased food consumption on day 3. Bioanalytical analysis of plasma confirmed SRP-001 exposure in all animals. Overall, SRP-001 was tolerated when administered 3 times daily for 2 d at 900 mg/kg/d, and plasma exposure was observed.

7) A Dose-Range-Finding and 7-day Repeat-Dose Oral Toxicity Study of SRP-001 in Beagle dogs (Non-GLP) In Phase II of this study, 0, 30, 100, or 300 mg/kg/d of SRP-001 was administered once daily for seven consecutive days to 1 Beagle dog/sex/dose (Calvert Labs/Altasciences, Scott Township, PA USA). Mortality/morbidity was observed twice daily and once prior to scheduled sacrifice. Clinical observations were evaluated prior to each dose administration and at approximately 1-3 h post-dose and once prior to scheduled sacrifice on day 8. Body weights were recorded on days 1 and 7, and a fasted body weight was recorded prior to scheduled sacrifice on day 8. Food consumption was recorded daily. Blood for evaluation of hematology, coagulation, and clinical chemistry parameters and urine for urinalysis was collected from all animals prior to treatment initiation and from all Phase II animals prior to scheduled sacrifice on day 8. Blood for toxicokinetic evaluation was collected at selected time points on days 1 and 7. Selected tissues were harvested and weighed at necropsy. No microscopic evaluations were performed. Clinical signs observed included loose or soft feces and emesis. While soft or loose feces were observed at similar frequencies in all animals, emesis was observed at a higher incidence and greater volume in animals that received higher dose levels. The clinical observations corroborated with findings of red discoloration in the duodenum of the control group female, the jejunum of the low-dose male, and the colon of both high-dose dogs. No SRP-001-related differences in body weight, food consumption, hematology, clinical chemistry, or coagulation parameters were observed.

AUC and Cmax levels increased in a dose-dependent manner, with day 1 levels higher than those from day 7. The reason for this is unclear; however, it may be due to a low sample size (n=2 per dose group). Half-lives ranged from approximately 1 to 3.5 h and increased with dose and time. Tmax ranged from 0.5 to 1.5 h. Based on these observations, SRP-001 was considered to be tolerated at 300 mg/kg when administered once daily for 7 consecutive days. Our toxicokinetic analysis indicates elevated systemic exposures to SRP-001 on Day 1 relative to Day 7. This trend is corroborated by a decline in both AUC and Cmax values on Day 28, aligning with our prior 28-day rat toxicology study that reported analogous reductions in systemic exposures at daily dosages of ≥1500 mg/kg/day. This is further supported by human pharmacokinetic data from the Phase 1 MAD cohort, which also demonstrated a decrease in SRP-001's PK parameters on Day 5 versus Day 1. A plausible mechanistic explanation for this observed decrease is the induction of metabolic enzymes, likely involving UGT and potentially CYP3A enzymes, as elaborated above and substantiated by previous research. While emesis was observed, its frequency did not increase over time, making it an unlikely factor in the reduced systemic exposure on Day 28. Moreover, no emesis was reported in the Phase 1 MAD cohort, which also showed a PK parameter decrease on Day 5 compared to Day 1. The NOAEL exposure parameters align well with the highest SAD dose in the Phase 1 trial, reinforcing the drug's safety profile. The observed PK decrease is not attributed to emesis but likely to enzymatic induction and will be the subject for future investigation.

8) A 28-day Oral Toxicity Study of SRP-001 in Beagle Dogs with a 14-day Recovery (GLP study) SRP-001 was supplied by Olon Ricerca Bioscience as an off-white powder. The test article was then prepared into dosing suspensions for oral administration via gavage (Calvert Labs/Altasciences, Scott Township, PA USA). Thirty-two experimentally naïve Beagle dogs (16 males and 16 females), 8-9 months old and weighing 6.7-10.5 kg for males and females at the outset of the study, were administered 0, 165, 330, or 495 mg/kg/d. Animals were dosed three times daily (3-5 h between doses) for 28 consecutive days. Prior to this dosing schedule, the dosing was initiated with once daily dosing at 0, 100, 300, or 900 mg/kg at a dose volume of 5 to 10 ml/kg. Due to adverse clinical signs noted following dosing of all groups at a dose volume of 10 ml/kg, dosing of the animals was suspended on day 2 for males and day 1 for females. The study was restarted with T.I.D. dosing in lower dose volume to reduce vehicle-related toxicity.

In-life data from the initial start of the study using once daily dosing were not included in the report but are maintained in the study file. Following the restart of the study, the study was conducted as follows. Mortality/morbidity was performed twice daily and once prior to the scheduled sacrifice. Animals were observed prior to each dose administration and 1-3 h after each dose administration. Animals were also observed once daily on non-dosing days and once prior to scheduled sacrifice. Body weights were recorded prior to randomization/selection, prior to the first dose administration on day 1 and weekly thereafter. Food consumption was determined daily. Ophthalmology examinations and electrocardiograms were performed before treatment initiation and during the last week of dosing. Blood for evaluation of hematology, coagulation and clinical chemistry and urine for urinalysis was collected prior to treatment initiation and on days 29 and 43. Blood for toxicokinetic evaluation was collected at selected timepoints on days 1 and 28. All surviving animals were sacrificed on day 29 or day 43. Selected tissues were harvested at necropsy, selected organs weighed, and selected tissues evaluated microscopically. SRP-001-related clinical signs included emesis, loose feces, and salivation. While emesis and loose feces were observed in all dose groups, including the control, they were observed at a higher frequency and severity with increasing dose levels.

Assessment of hematology parameters identified SRP-001-related decreases in red blood cells, hemoglobin, hematocrit, and mean corpuscular hemoglobin concentration and increases in mean corpuscular volume and absolute and relative reticulocytes. These changes correlated with increased erythroid cellularity of the bone marrow and extramedullary hematopoiesis of the spleen and liver, indicative of regenerative anemia. Due to the limited magnitude of the changes in hematology parameters and the corresponding changes in reticulocytes, this finding was not considered adverse.

Assessment of clinical chemistry parameters indicated SRP-001-related increases in triglycerides and alkaline phosphatase and decreases in cholesterol. Increases in alkaline phosphatase correlated with bile accumulation in the liver of Group-4 males. This finding was indicative of the biliary system starting to be overwhelmed and was considered adverse. Other microscopic findings of the liver included Kupffer cell pigment, Kupffer cell erythrophagocytosis, and Kupffer cell hypertrophy/hyperplasia. The Kupffer cell changes were thought to be, at least in part, due to phagocytosis of erythrocytes resulting in the accumulation of cytoplasmic pigment and increased demand for Kupffer cells resulting in hypertrophy/hyperplasia. These microscopic changes correlated with increased liver organ weights. Microscopic evaluation also identified thyroid gland follicular cell hypertrophy.

No SRP-001-related changes were observed in body weights, food consumption, ophthalmology examination, electrocardiology examination, coagulation parameters, or urinalysis parameters. Analysis of dogs following a 14-d recovery period indicated that nearly all SRP-001-related changes observed on day 29 were reversible. Only microscopic changes in Kupffer cell pigment and erythrophagocytosis and minimal increased erythroid cellularity of the bone marrow were still present in Group-4 animals following the recovery period. Exposure to SRP-001 was dose-dependent and generally dose-proportional to greater than dose-proportional from 165 to 330 mg/kg/d and less than dose-proportional from 330 to 495 mg/kg/d. Exposure to SRP-001 following 28 consecutive days of oral dosing was 2 to 5 times less than exposure observed on day 1. Based on the clinical signs, microscopic changes, and clinical pathology changes, the no-adverse-effect level for SRP-001 was 330 mg/kg/d when given T.I.D. for 28 d.

### Oral Nanosuspension Formulation for Human Use and Determination of the Optimal Formulation Stability

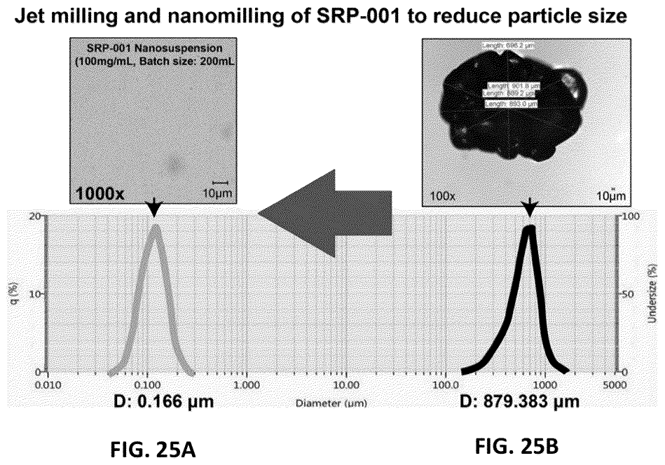

The drug therapeutic candidate is manufactured in a two-step process (Latitude Pharmaceuticals, Inc., San Diego, CA USA). First, SRP-001 is jet milled. Second, the milled API is dispersed in a solution comprising 1% hydroxypropyl cellulose (1% HPC) and sterile water for injection and subjected to wet milling. The target particle size is approximately D₉₀ 0.15 μm. Hence, using nanomilling and jet-milling techniques, we identified the optimal solvent in which to dissolve to develop SPR-001 as an oral nanoparticle suspension containing 100 mg/mL SRP-001 in preserved aqueous 1% HPC (FIG. 25). An oral nanoparticle suspension containing 100 mg/mL SRP-001 in aqueous 1% HPC preserved with 0.1% sodium benzoate was manufactured for use in nonclinical studies as Lot LPI2021028 and stored at 5° C.±3° C. All specifications were met at time 0, 1 month, and 3 months at 5° C. and 25° C./60% RH (relative humidity). The same formulation as above was manufactured under cGMP for use in clinical studies as Lot LPI2021031 and stored at 5° C.±3° C. All specifications were met. This lot has been placed on stability at 5° C., 25° C./60% RH, and 40° C./75% RH. Finally, an appearance-matched placebo consisting of 2% microcrystalline cellulose in aqueous 1% HPC preserved with 0.1% sodium benzoate and 0.05% EDTA disodium dihydrate was manufactured under cGMP for use in clinical studies. The above oral suspensions were manufactured by Latitude Pharmaceuticals Inc. (San Diego, CA, USA). Furthermore, the active pharmaceutical ingredient (API, SRP-001) manufacturer, Olon Ricerca Biosciences, demonstrated that the API is stable for at least 9 months at 25° C./60% RH and 6 months at 40° C./75% RH.

### Human Studies

A two-part randomized, double-blind, placebo-controlled Phase 1 trial was conducted to assess the safety, tolerability, PK and PD of SRP-001 ascending doses (Quotient Sciences, Miami, FL USA). The study was conducted in accordance with the International Council for Harmonisation of Technical Requirements for Pharmaceuticals for Human Use (ICH), Guideline for GCP: Consolidated Guidance (E6), and applicable regulatory requirements(s) including clinical research guidelines established by the Basic Principles defined in the U.S. 21 CFR Parts 50, 56, and 312 and the principles enunciated in the Declaration of Helsinki (revised version Fortaleza 2013). The clinical protocol and informed consent form were reviewed and approved by an Institutional Review Board (IRB; Advarra IRB, 6100 Merriweather Drive, Suite 600, Columbia, MD 21044; Quotient Protocol No. SRP-101 and Quotient Sciences Study No. QSC205130; Quotient Sciences Miami, 3898 NW 7th Street, Miami, FL 33126.). The study comprises a SAD (Part 1) assessment that includes a food effect assessment that contributes data to inform a subsequent MAD (Part 2) dose-ranging study. Safety measurements were collected throughout the study for all subjects. Blood samples were collected to determine the PK parameters of SRP-001. ClinicalTrials.gov identifier: NCT05484414

Inclusion criteria: The normal healthy volunteer (NHV) subjects are healthy adult male or female subjects, 18-55 years of age inclusive, with a body mass index of between 18.0 and 32.0 kg/m² (inclusive) as measured at screening or, if outside the range, considered not clinically significant by the Investigator, and weighs within +15% of the desired weight at screening, according to the 1993 Metropolitan Life Insurance Table. Females must not be pregnant or breastfeeding. The subjects must be capable of understanding the Informed Consent Form and agreeing to comply with the requirements of the study during its duration. The subjects are to be in good health as determined by medical history, physical examination, clinical laboratory test results, and 12-lead ECG. Females must be of non-childbearing potential (defined as surgically sterile (i.e., had a bilateral tubal ligation, bilateral salpingectomy, hysterectomy, or bilateral oophorectomy) or post-menopausal for at least 1 year before the first dose of study medication) or agree to use an acceptable form of birth control from screening until 31 days after last SRP-001 administration. Male subjects must either be surgically sterile (vasectomy at least 3 months prior to first dose) or agree to the use of a birth control method such as a condom with spermicide from screening until 91 days after last SRP-001 administration. All subjects must also be willing and able to remain in the research center for the entire duration of the confinement period and return for the outpatient visits; and, have vital signs (measured after the subject has been in a supine position for a minimum of 5 min) at screening within the following ranges: heart rate: 40-100 bpm; systolic blood pressure (BP): 90-145 mmHg; diastolic BP: 50-95 mmHg. Out-of-range vital signs may be repeated once.

Exclusion criteria: NHVs were excluded from study participation for any of the following. A history or presence of clinically significant cardiovascular, pulmonary, hepatic, renal, hematologic, GI, endocrine, immunologic, dermatologic, neurologic, oncologic, or psychiatric disease or any other condition that, in the opinion of the Investigator, would jeopardize the safety of the subject or the validity of the study results (a clinically significant illness within 30 days preceding the screening visit). If he or she had been on a significantly abnormal diet during the 4 weeks preceding the first dose of study medication. Subjects who have received any investigational product in a clinical research study within 5 half-lives or within 30 days prior to first dose. However, in no event shall the time between the last receipt of investigational product and the first dose be less than 30 days. Any NHVs who have previously been administered SRP-001 in this study. Subjects who are taking, or have taken, any prescribed drug (other than hormone replacement therapy/hormonal contraception) in the 14 days before study medication, or OTC drug or herbal remedies in the 72 h before study medication. COVID-19 vaccines are accepted concomitant medications. NHVs were also excluded if they had been treated with any known drugs that are moderate or strong inhibitors/inducers of cytochrome P450 (CYP) enzymes (e.g., barbiturates, phenothiazines, cimetidine, carbamazepine) within 30 days before the first dose of study medication, and that, in the Investigator's judgment, may impact subject safety or the validity of the study results; if they had a history of hypersensitivity (has developed an allergic reaction) to acetaminophen or similar chemical entities; or presence or history of clinically significant allergy requiring treatment, as judged by the Investigator. The NHV's hemoglobin concentration and hematocrit should be within 5% of normal before participating in the study. It is recommended that blood/plasma donations not be made for at least 30 days after discharge from the study. Current smokers and those who have smoked within the last 12 months were also excluded, as anyone with a confirmed positive urine cotinine test at screening or admission; current users of e-cigarettes and nicotine replacement products and those who have used these products within the last 12 months; consumption of beverages or foods that contain alcohol, grapefruit, poppy seeds, broccoli, Brussels sprouts, pomegranate, star fruit, char-grilled meat, or caffeine/xanthine from 48 h before the first dose of study medication until discharge from the study. NHVs were instructed not to consume any of the above products. Also excluded is an NHV with a confirmed positive alcohol urine test at screening or admission; a female with a positive pregnancy test result; or a positive urine screen for drugs of abuse (amphetamines, barbiturates, benzodiazepines, cocaine, cannabinoids, or opiates); a positive test for hepatitis B surface antigen (HBsAg), hepatitis C antibody (HCV Ab), or human immunodeficiency virus (HIV) at screening or has been previously treated for hepatitis B, hepatitis C, or HIV infection. Anyone with the presence of active infection, mucositis, cold sores, aphthous ulcers, vesicles, viral lesions, local irritation/inflammation, or periodontal disease of the oral cavity was also excluded, as well as NHVs with a known glucose-6-phosphate-dehydrogenase (G6PD) deficiency.

Subject randomization scheme: NHVs were assigned a three-digit screening number after informed consent was obtained (e.g., 001, 002, 003, etc.). Four-digit subject numbers were allocated on the morning of dosing according to the code 1001 to 1020 for males and 1021 to 1040 for females using the lowest number available. Replacement subjects were allocated subject numbers 9001 to 9020 for males and 9021 to 9040 for females, where the last three digits are the same as those of the original subject (e.g., if Subject 1005 withdraws, the replacement will have the Subject Number 9005 and will receive the same regimen as Subject 1005. In addition, for the 900 mg fed cohort, subjects received the same regimen as the withdrawn subject in both the fasted and fed states.

Blinding: Pharmacy staff—An unblinded pharmacy staff was required at the Clinical Site to comply with the study's randomization and blinding requirements. At the clinical research unit, prior to study initiation, the Principal Investigator was responsible for designating a qualified pharmacy staff to serve as the unblinded pharmacy staff in the study. Unblinded pharmacy staff could dose subjects but could not participate in any subject assessments. Throughout the study, the designated unblinded pharmacy staff was responsible for all drug accountability issues, including preparing, labeling, dispensing, and dosing study drug according to the randomization code provided, yet remain independent of all subject assessments. The pharmacy staff followed the standard operating procedures and Work Instructions related to pharmacy services and protocol-specific requirements. Randomization codes were provided to the unblinded pharmacy staff. Confirmation of receipt of the randomization code was required by the Sponsor. The Principal Investigator was ultimately responsible for ensuring that the integrity of the blind is maintained throughout the study at the site and required to notify the Safety Review Committee in the event of any breaking of the blind for any reason. Clinical Research Staff—All observers who evaluate any reported AE, laboratory abnormalities, ECGs, and changes in the ECGs were blinded as to what treatment each subject is assigned. Study Subjects—All subjects were blinded as to which treatment they received and dose. Data Sciences Staff—The unblinded statistician was not involved in any decisions relating to populations for analysis prior to unblinding. Interim PK parameter estimations were performed using bioanalytical data applied with subject aliases to maintain the study blinded. Further, the Data Sciences department did not have access to the randomization schedule before database lock and unblinding. Pharmacovigilance—A copy of the randomization schedule was made available to the pharmacovigilance provider for analysis of pharmacovigilance. Bioanalytical Laboratory—All samples were sent to the bioanalytical laboratory for analysis. The bioanalytical laboratory was unblinded, a copy of the randomization schedule provided, and only ran the analysis on active treatment subjects. Pharmacokinetic Analysis—PK analysis was completed only on the active treatment subjects and blinded by subject aliases for interim assessments.

Check-In Procedures: At each check-in all subjects were evaluated to confirm they continue to meet all the inclusion criteria and none of the exclusion criteria. General risk mitigation against COVID-19 was implemented in accordance with Quotient Sciences' monitoring and prevention control measures. A urine sample was collected from all subjects at each study check-in to screen for drugs of abuse and alcohol. If at any time an alcohol or drug test is positive, the subject is discontinued from study participation. Female subjects of childbearing potential and male subjects will be asked to confirm that they still adhere to the contraceptive criteria. A serum pregnancy test was performed on all female subjects at each check-in using the clinical chemistry blood sample; this test had to be negative for the subject to continue study participation. Blood and urine samples were also collected at admission for clinical laboratory assessments.

Confinement: NHVs were admitted to the research center in the morning of the day before study drug administration (Day −1) to ensure a minimum 10-h fast before dose administration for the fasted regimens. Subjects enrolled in the study had remained in the research center until the completion of the study procedures scheduled for 48 h after dose administration (Day 3), and they returned to the research center for 3 outpatient visits (Days 4, 5, and 6).

Follow-Up: A follow-up phone call took place between Days 7 and 10 (+2 days) to ensure the ongoing well-being of the subjects. If a subject reported any AEs which could present a cause for concern, they were required to attend the clinical unit for a further follow-up assessment (as an unscheduled visit) and were followed up until the AE had resolved. Completion of the last follow-up call or unscheduled follow-up visit was considered the end of the study.

Fasting/Meals/Beverages: An optional meal or snack was served the evening of check-in (Day −1). NHVs were allowed water up to 1 h before the scheduled dosing time and were provided with 240 mL of water at 1 h post-dose. Water was allowed ad libitum after 1 h post-dose. Decaffeinated fluids were allowed ad libitum from lunchtime on the day of dosing. Decaffeinated fluids were allowed ad libitum from lunchtime on the day of dosing until 1 h before and then from 1 h after the afternoon and evening dose.

Fasted and Fed Dosing: NHVs were dosed in the fasted state. The calorie/fat content of meals was not required to be controlled. Subjects were provided with a standardized menu. Subjects were provided with a light snack and then fasted from all food and drink (except water) for a minimum of 10 h on the day prior to dosing until approximately 4 h post-dose, at which time lunch was provided. An evening meal was provided at approximately 10 h post-dose/post-morning dose and an evening snack at approximately 14 h post-morning dose. Food effect was studied in the cohort receiving 900 mg SRP-001 in the fed state. A standard high-fat breakfast was given 30 min before dosing. On Day −1, these NHVs were provided with a light snack and fasted from all food and drink (except water) until the following morning, when they were provided with a standard high-fat breakfast. The breakfast had to be consumed over a maximum period of 25 min, with dosing occurring 30 min after the start of breakfast. Subjects were encouraged to eat their meal evenly over the 25 min period. It was acknowledged that some subjects took less time to eat, but dosing still occurred 30 min after the start of breakfast. Subjects had to consume at least 90% to be eligible for dosing. The start and stop time and percentage of the breakfast consumed were recorded in the source. Acceptable deviations for the pre-dose meal from the nominal time point were: 1) Pre-dose meal was provided within +5 min of the nominal time point, 2) Lunch was provided at approximately 4 h post-dose, 3) an evening meal at approximately 10 h post-dose, and 4) an evening snack at approximately 14 h post-dose. On subsequent days, meals were provided at appropriate times.

Safety—Holter (continuous ECG) Monitoring: NHVs also underwent continuous ECG Holter monitoring from at least 1 h prior to dosing until 24 h post-dose, and resulting measurements were monitored consistently. All ECGs were collected electronically using a Mortara H12+Holter monitor. Specific procedures for ECG Holter recording and extractions were provided to the Investigator by the Central ECG Laboratory, Banook Group. On Day 1, continuous Holter recording commenced at least 1 h prior to dosing until 24 h post-dose. Subjects were required to rest in the supine position with no external stimuli (e.g., music or television) for 15 min prior to the nominal time point where an ECG extraction was scheduled. This extraction was taken by the third-party Holter provider at the planned time, and therefore, no other procedures were performed within the resting period. Where one period of ECG rest ran into the rest period for the next ECG extraction, the minimum resting period before ECG extraction did not need to be started again except in cases where the rest was disturbed (e.g., subjects had to get up for a comfort break after the preceding extraction). Subjects were allowed to move more freely outside of the primary ECG rest periods. Comfort breaks for hygienic purposes were allowed each morning well in advance of the required supine/ECG extraction points. Where any scheduled rest times were missed, where there was less than a 10-min rest period, or where the final 5 min of a rest period was interrupted (for example, by subject movement or loss of leads), a protocol deviation was recorded, and the Holter provider was informed. Loss of leads or interruption of the recording outside of the scheduled rest times was not considered a protocol deviation but was recorded in the source. The acceptable deviations for ECG rest periods from the nominal time point were pre-dose and post-dose ECG resting periods ending within ±15 min of the nominal time point. ECGs were collected electronically and over-read by cardiologists at the Central ECG Laboratory, Banook Group. The Central ECG Laboratory over-read was used for data analysis and report writing purposes.

Adverse Event: An AE was defined as any untoward medical occurrence associated with the use of a drug in humans, whether or not considered drug-related. An AE could be any unfavorable and unintended sign (e.g., an abnormal laboratory finding), symptom, or disease temporally associated with the use of a drug, without judgment to causality. An AE could arise from any use of the drug (e.g., off-label, use in combinations with another drug) and from any route of administration, formulation, or dose, including an overdose.

Recording Adverse Events: AEs were recorded from the time of providing written informed consent until 30 days after the last dose of study drug. During each study visit, the subject was questioned directly regarding the occurrence of any adverse medical event according to the schedule in eCRF. All AEs, whether ascribed to study procedures or not, were documented immediately in the subject's eCRF. This included the date and time of onset, a description of the AE, severity, seriousness, duration, actions taken, outcome, and an Investigator's current opinion on the relationship between the study drug and the event. A diagnosis and final opinion on the relationship between the study drug and the event were provided at the end of the study by the Quotient Sciences Principal Investigator. Any subject who withdrew from the study due to an AE was followed up until the outcome was determined and written reports were provided by the Investigator.

Life-Threatening Adverse Event/Life-Threatening Suspected Adverse Reaction: A life-threatening AE/life-threatening suspected adverse reaction, in the view of either the Investigator or Sponsor, placed the patient or subject at immediate risk of death. It did not include an adverse reaction that, had it occurred in a more severe form, might have caused death. None of these occurred in the study. Any life-threatening AE was reported and managed as an SAE.

Serious Adverse Event/Serious Suspected Adverse Reaction: A SAE or serious suspected adverse reaction, in the view of the Principal Investigator, resulted in any of the following outcomes: Death, a life-threatening AE, inpatient hospitalization or prolongation of existing hospitalization, a persistent or significant incapacity or substantial disruption of the ability to conduct normal life functions, or a congenital anomaly/birth defect. Important medical events that might not result in death, be life-threatening, or require hospitalization could be considered serious when, based upon appropriate medical judgment, they might jeopardize the patient or subject and might require medical or surgical intervention to prevent one of the outcomes listed in this definition.

Unexpected Adverse Event/Unexpected Suspected Adverse Reaction: An unexpected AE/unexpected suspected adverse reaction was an AE or suspected adverse reaction that was not listed in the FDA Investigator Brochure (IB) or was not listed at the specificity or severity that had been observed; or, was not consistent with the risk information described in the general investigational plan or elsewhere in the current application, as amended.

Grading of Adverse Events: The severity of AEs was assessed as follows: Mild—An AE that was easily tolerated by the subject, caused minimal discomfort, and did not interfere with everyday activities. Moderate—An AE that was sufficiently discomforting to interfere with normal everyday activities; intervention might have been needed. Severe—An AE that prevented normal everyday activities; treatment or other intervention was usually needed. Relationship to Study Treatment—The relationship between the AE and the investigational product was determined by the Principal Investigator or Sub-Investigator on the basis of their clinical judgment and the following definitions: Related: The AE followed a reasonable temporal sequence from the study product administration and could not be reasonably explained by the subject's clinical state or other factors (e.g., disease under study, concurrent diseases, or concomitant medications). The AE followed a reasonable temporal sequence from the study product administration and represented a known reaction to the drug under study or other drugs in its class or was predicted by the known pharmacological properties of the drug. The AE resolved with discontinuation of the investigational product and/or recurred with rechallenge, if applicable. Not Related: The AE did not follow a reasonable temporal sequence from study product administration or could be reasonably explained by the subject's clinical state or other factors (e.g., disease under study, concurrent diseases, and concomitant medications).

SRP-001 administration and PK/PD determination: Following a single administration of 300-2,000 mg SRP-001 oral suspension in the fasted state (and in cohort 4, 900 mg in the fed state), plasma concentrations of SRP-001 were observed from the first sampling timepoint of 1 h post-dose for all subjects. Peak concentrations (Tmax) were also reached at 1 h post-dose for all subjects, after which concentrations declined in a mono- or bi-phasic manner and remained quantifiable up to between 4 to 12 h postdose, giving rise to a geometric mean T_{1/2} of 2.38 h. Maximum and overall plasma exposure of SRP-001 based on geometric mean (geometric CV %) Cmax, AUC(0-48) and AUC(0-inf) were 665 ng/mL (55.5%), 1630 ng·h/mL (37.6%) and 1630 ng·h/mL (37.4%) respectively.

Following a single administration of 600 mg SRP-001 oral suspension in the fasted state, plasma concentrations of SRP-001 were observed from the first sampling timepoint of 1 h post-dose for all subjects. Peak concentrations (Tmax) were reached between 1 and 2 h post-dose for all subjects, after which concentrations declined in a mono- or bi-phasic manner and remained quantifiable up to between 8 to 24 h post-dose, giving rise to a geometric mean T1/2 of 5.04 h. Maximum and overall plasma exposure of SRP-3) based on geometric mean (geometric CV %) Cmax, AUC(0-24) and AUC(0-inf) were 1740 ng/mL (48.7%), 5660 ng·h/mL (25.3%) and 5840 ng·h/mL (29.5%), respectively. increasing the dose from 300 to 600 mg also increased geometric mean Cmax and AUC(0-24) values by 2.6- and 3.5-fold.

Following a single administration of 900 mg SRP-001 oral suspension in the fasted state, plasma concentrations of SRP-001 were observed from the first sampling time-point of 1 h post-dose for all subjects. Peak concentrations (Tmax) were reached at 1 h post-dose for all subjects, after which concentrations declined in a mono- or bi-phasic manner and remained quantifiable up to between 12 to 24 h post-dose, giving rise to a geometric mean T1/2 of 3.26 h. Maximum and overall plasma exposure of SRP-3D (DA) based on geometric mean (geometric CV %) Cmax, AUC(0-24) and AUC(0-inf) were 2700 ng/mL (35.1%), 6870 ng·h/mL (39.0%) and 6970 ng·h/mL (39.5%), respectively. Increasing the dose from 600 to 900 mg also increased geometric mean Cmax and AUC(0-24) values by 1.6- and 1.2-fold.

Following a single administration of 900 mg SRP-001 oral suspension in the fed state, plasma concentrations of SRP-001 were observed from the first sampling time-point of 1 h post-dose for all subjects. Peak concentrations (Tmax) were reached between 1 and 8 h post-dose, after which concentrations declined in a mono- or bi-phasic manner and remained quantifiable up to between 12 and 24 h post-dose, giving rise to a geometric mean T1/2 of 3.79 h. Maximum and overall plasma exposure of SRP-001 based on geometric mean (geometric CV %) Cmax, AUC(0-24) and AUC(0-inf) were 1200 ng/mL (32.4%), 6250 ng·h/mL (22.3%) and 5910 ng·h/mL (17.1%), respectively. The relative bioavailabilities based on geometric mean Cmax, AUC(0-24), AUC(0-last) and AUC(0-inf) were 48.4% (21.9%), 99.2% (19.0%), 98.4% (16.3%) and 101% (20.3%), respectively. In a two-day oral dose toxicity and pharmacokinetic (PK) study of SRP-002 in Beagle dogs, peak plasma concentrations of SRP-001 were detected within a 30-minute to 1-hour window post-administration. Maximal plasma concentrations ranged from 3654-4879 ng/mL in the low-dose cohort and 9760-17003 ng/mL in the high-dose cohort. Intriguingly, a nine-fold escalation in dose between the low and high-dose groups yielded only a 3-4-fold amplification in peak plasma concentration. This observation underscores a dose-dependent relationship in both AUC_{0-inf} and Cₘₐₓ values, in accordance with established PK principles. These results are congruent with the known PK behavior of compounds within this class.

Finally, PKs from the last SAD cohort (2,000 mg fasted state) demonstrate that following SRP-001 oral suspension, plasma concentrations of SRP-001 are observed from the first sampling time-point of 1 h post-dose for all subjects. Peak concentrations (Tmax) were reached at 1 or 2 h post-dose, after which concentrations declined in a mono- or bi-phasic manner and remained quantifiable up to between 24 to 48 h post-dose, giving rise to a geometric mean T1/2 of 5.96 h. Maximum and overall plasma exposure of SRP-001 based on geometric mean (geometric CV %) Cmax, AUC(0-24) and AUC(0-inf) were 4590 ng/mL (34.0%), 18900 ng·h/mL (25.7%) and 19600 ng·h/mL (25.7%), respectively. Increasing the dose from 900 to 2000 mg resulted in a sub-proportional increase in geometric mean Cmax and AUC(0-24) values, with fold-increases of 1.7- and 2.8-fold, respectively, for the 2.2-fold increase in dose (FIG. 3H-3I). The geometric mean Cmax and AUC(0-24) values represented 34% and 31% of the exposure limits defined in the protocol, respectively.

### Statistics

Changes in the withdrawal thresholds or latencies induced by a drug were first analyzed with a one-way ANOVA. Comparisons between the effects of different drugs were then subjected to t-test for unpaired means. A value of p<0.05 was considered significant. All statistical analyses were performed using GraphPad Prism 9.5.1. Missing data points in the statistical analysis were addressed using Multiple Imputation, a robust technique that effectively mitigates missing values and minimizes bias. This methodology aligns with contemporary best practices and guidelines for clinical trials. In contrast to the Last Observation Carried Forward (LOCF) method, which is prone to introducing bias, Multiple Imputation offers a more faithful representation of the data distribution.

Single-cell (scRNA) RNA sequencing. For cell nuclei isolation from the midbrain PAG, they were flash-frozen, dissected (by a cryotome), minced into small pieces, homogenized, filtered through 70 strainer and centrifuged. ReadiDrop 7-AAD cell viability dye (Bio-Rad) added, and flow sorted the live (7-AAD positive) nuclei in a BioRad Cell Sorter S3e using purity mode. Nuclei were counted using Nexcelom automated Cellometer with acridine orange/propidium iodide (AOPI) stain, and the Nuclei Stock Concentration used so that 10,000 nuclei could be targeted for each sample. Samples were bulk transposed and around 16,100 nuclei were loaded onto each channel of the microfluidic chip and GEMs generated using the 10× Chromium controller, with the reagents from the Chromium Next GEM Single Cell ATAC Kit, 1000280 (10× Genomics), following manufacturer instructions, followed by post-GEM cleanup with Dynabeads and SPRIselect (Beckman Coulter). Then after pre-amplification PCR, samples were divided for the ATAC and for the GEX library construction. Post library construction QC was performed using Agilent Bioanalyzer 2100 on High Sensitivity DNA chips, and after verification of sample traces, each library normalized based on the average fragment size, and the concentration determined using Qubit 4.0 fluorometer (Thermo Fisher Scientific). Pooled Libraries were then sequenced by using Illumina high output kits on a NovaSeq 6000 platform.

Data processing. Cell Ranger ARC (cellranger-arc) v.2.0.2 (10×Genomics) was used to process Chromium Single Cell Multiome ATAC+Gene Expression (GEX) sequencing data. Briefly, raw BCL files from the Illumina NovaSeq were demultiplexed into paired-end, gzip-compressed FASTQ files that were generated using cellranger-arc mkfastq and default parameters. Read alignment, filtering, barcode counting, peak calling, and counting of both ATAC and GEX molecules were performed using cellranger-arc count. The rat genome mRatBN7 and its annotated transcriptome were used as the reference for alignment. Only confidently mapped reads with valid barcodes, unique molecular identifiers (UMIs), and non-PCR duplicated were retained. The overall sequencing quality was evaluated by looking at the summary metrics of the web_summary.html file generated for each sample. Processed data sets of multiple samples were then aggregated with cellranger-arc aggr to normalize input runs to the same median fragments per cell across samples. The linkage between chromatin accessibility and GEX was also established for each sample with this tool, as the ATAC and GEX measurements are on the very same cell.

### Seurat and Single Cell Downstream Analysis

Standard pre-processing through the Seurat workflow was performed for single-cell RNAseq data. Mitochondrial genes were identified, and cells expressing >5 percent of mitochondrial features were removed. The dataset was log normalized with a size factor of 10,000 molecules for each cell using the NormalizeData function. Z-score transformation was performed across all cells to standardize expression values for genes using the ScaleData function. Highly variable features for each group were identified using the FindVariableFeatures function. The top 10 variable features were plotted.

To visualize the data principal component analysis (PCA) was performed for dimensionality reduction, PCA dimensions were further reduced into two dimensions using UMAP. Weighted shared nearest neighbor and clustering of single cells was performed by Louvain algorithm. A table of logged 2-fold change and p values between each treatment group was calculated with Seurat. This calculation determines differential expression using non-parametric Wilcoxon rank sum test; these values were used in heatmaps and dot plots showing differential expression of top 50 DE and pain-related genes. Additionally, the top 50 differentially expressed genes were used in Gene Ontology enrichment analysis described below.

Cell type annotation was performed with the ScType package, which utilizes the largest database of human and mouse-specific cell markers to date. The database was compiled by integrating CellMarker and PanglaoDB databases and manually curated 15 novel cell types by identifying marker genes from publications. The automatic cell annotations were based on clusters identified using Seurat PCA and UMAP, then integrated with the Seurat dataset for downstream analysis and visualization.

Gene Ontology Enrichment Analysis. Enrichment analysis was performed on the top 50 differentially expressed genes from comparisons between Vehicle vs CFA, CFA vs CFA_ApAP, and CFA vs CFA_SRP_001. The gene sets generated were used to determine the enrichment of GO categories, with R package clusterProfiler. Visualization of the enrichment result object was achieved with R packages DOSE and enrichplot. Visualization included: Barplots showing the enriched terms and corresponding enrichment score (p-values) and gene count, gene concept networks showing linkage of genes and GO terms, and enrichment maps showing the enriched terms organized into a network connecting overlapping gene sets.

Qiagen Ingenuity Pathway Analysis (IPA). The genes with known gene IDs and their corresponding DESeq2 expression values were uploaded into the software using the batch upload feature. Each gene symbol was mapped to its corresponding gene object in the Ingenuity Pathways Knowledge Base. Unbiassed pathways and gene networks were algorithmically generated based on their connectivity and assigned a score. The score considers the number of focus genes in the network and the size of the network to approximate how relevant this network is to the original list of genes. The networks identified are then presented as a graph indicating the molecular relationships between genes/gene products. Upstream causal networks indicating potential/predicted upstream relationships based on the input gene list are also generated, as well as enrichment plots based on the most significant/highest-scoring pathways containing genes from the uploaded dataset. The “path designer” module was used to polish the network images. Data from IPA for canonical pathway enrichment was exported, and histograms were produced using RStudio 2021.09.0.

### Cell-Cell Communication Analysis

To study intercellular communication, we applied the CellChat (version 1.5.0) R package. First, the expression data, together with the identified cell clusters, were converted into a CellChat object using the “createCellChat” function. Known ligand-receptor pairs were obtained from the CellChatDB. Prior to running CellChat, the “subsetCellChat” function was used to filter out genes that were not included in the “CellChatDB”. Then, the “identifyOverExpressedGenes” and “identifyOverExpressedlnteractions” functions were applied to identify cluster-specific ligand and receptor genes. The “computeCommunProb” function was utilized to calculate the communication probabilities among different cell clusters. We aggregated these probabilities using the “aggregateNet” function. To illustrate variations in the intensity of cell-to-cell communication, we utilized the “netVisual_diffinteraction” function. Following this, we employed non-negative matrix factorization (NMF) through the “identifyCommunicationPatterns” function to explore the underlying coordination among multiple cell clusters and signaling pathways. This helped us infer the number of distinct patterns governing intercellular communication. River plots were used to visualize the major signaling inputs and outputs among each of the cell types. The following references are hereby incorporated by reference.

### REFERENCES

1. Zimmer, Z., Fraser, K., Grol-Prokopczyk, H. & Zajacova, A. A global study of pain prevalence across 52 countries: examining the role of country-level contextual factors. Pain 163, 1740-1750 (2022).
2. Henschke, N., Kamper, S. J. & Maher, C. G. The epidemiology and economic consequences of pain. Mayo Clin Proc 90, 139-147 (2015).
3. Chang, A. K., Bijur, P. E., Esses, D., Barnaby, D. P. & Baer, J. Effect of a Single Dose of Oral Opioid and Nonopioid Analgesics on Acute Extremity Pain in the Emergency Department: A Randomized Clinical Trial. JAMA: the journal of the American Medical Association 318, 1661-1667 (2017).
4. Moore, P. A. & Hersh, E. V. Combining ibuprofen and acetaminophen for acute pain management after third-molar extractions: translating clinical research to dental practice. J Am Dent Assoc 144, 898-908 (2013).
5. Wininger, S. J. et al. A randomized, double-blind, placebo-controlled, multicenter, repeat-dose study of two intravenous acetaminophen dosing regimens for the treatment of pain after abdominal laparoscopic surgery. Clin Ther 32, 2348-69 (2010).
6. Lee, Y. et al. Intravenous Acetaminophen Versus Placebo in Post-bariatric Surgery Multimodal Pain Management: a Meta-analysis of Randomized Controlled Trials. Obes Surg 29, 1420-1428 (2019).
7. Van Aken, H., Thys, L., Veekman, L. & Buerkle, H. Assessing analgesia in single and repeated administrations of propacetamol for postoperative pain: comparison with morphine after dental surgery. Anesth Analg 98, 159-65, table of contents (2004).
8. Akil, A. et al. Paracetamol vs dexketoprofen for perineal pain relief after episiotomy or perineal tear. J Obstet Gynaecol 34, 25-8 (2014).
9. Bektas, F. et al. Intravenous paracetamol or morphine for the treatment of renal colic: a randomized, placebo-controlled trial. Ann Emerg Med 54, 568-74 (2009).
10. Qi, D. S. et al. A randomized, double-blind, placebo-controlled study of acetaminophen 1000 mg versus acetaminophen 650 mg for the treatment of postsurgical dental pain. Clinical therapeutics 34, (2012).
11. Larson, A. M. et al. Acetaminophen-induced acute liver failure: results of a United States multicenter, prospective study. Hepatology 42, 1364-72 (2005).
12. Rotundo, L. & Pyrsopoulos, N. Liver injury induced by paracetamol and challenges associated with intentional and unintentional use. World J Hepatol 12, 125-136 (2020).
13. Blieden, M., Paramore, L. C., Shah, D. & Ben-Joseph, R. A perspective on the epidemiology of acetaminophen exposure and toxicity in the United States. Expert review of clinical pharmacology 7, 341-348 (2014).
14. Mitchell, R. A. et al. Public awareness of acetaminophen and risks of drug induced liver injury: Results of a large outpatient clinic survey. PLoS One 15, e0229070 (2020).
15. Mendizabal, M. & Silva, M. O. Liver transplantation in acute liver failure: A challenging scenario. World J Gastroenterol 22, 1523-31 (2016).
16. Yoon, E., Babar, A., Choudhary, M., Kutner, M. & Pyrsopoulos, N. Acetaminophen-Induced Hepatotoxicity: a Comprehensive Update. Journal of Clinical and Translational Hepatology 4, 131 (2016).
17. Paracetamol pack limits Australia: Pack sizes will be reduced due to overdose rates, Therapeutic Goods Administration says in interim decision. https://www.9news.com.au/national/health-news-australia-tga-reduces-paracetamol-maximum-packet-size-interim-decision/48dc648f-0720-4f9a-bc3d-51e344e44340.
18. United Kingdom Medicines & Healthcare Products Regulatory Agency. MHRA Decision, September 2016: Multiple sales of anaglesic-containing in retail outlets; Best practice guidance on the sale of medicines for pain relief. (2016).
19. Antoniou, T., Guan, Q., Martins, D. & Gomes, T. Impact of acetaminophen product labelling changes in Canada on hospital admissions for accidental acetaminophen overdose: a population-based study. Canadian Medical Association Journal 194, E542-E548 (2022).
20. Nam, T. G. et al. Pyridine and pyrimidine analogs of acetaminophen as inhibitors of lipid peroxidation and cyclooxygenase and lipoxygenase catalysis. Org Biomol Chem 7, 5103-12 (2009).
21. Santoh, M. et al. Acetaminophen analog N-acetyl-m-aminophenol, but not its reactive metabolite, N-acetyl-p-benzoquinone imine induces CYP3A activity via inhibition of protein degradation. Biochem Biophys Res Commun 486, 639-644 (2017).
22. Streeter, A. J., Bjorge, S. M., Axworthy, D. B., Nelson, S. D. & Baillie, T. A. The microsomal metabolism and site of covalent binding to protein of 3′-hydroxyacetanilide, a nonhepatotoxic positional isomer of acetaminophen. Drug Metab Dispos 12, 565-76 (1984).
23. Shchepin, R. V. et al. Rational Design of Novel Pyridinol-Fused Ring Acetaminophen Analogues. ACS Med Chem Lett 4, 710-714 (2013).
24. Sahu, A. et al. Bioisosteric Replacement of Amide Group with 1,2,3-Triazoles in Acetaminophen Addresses Reactive Oxygen Species-Mediated Hepatotoxic Insult in Wistar Albino Rats. Chem Res Toxicol 33, 522-535 (2020).
25. Abdelmegeed, M. A., Moon, K.-H., Chen, C., Gonzalez, F. J. & Song, B.-J. Role of cytochrome P450 2E1 in protein nitration and ubiquitin-mediated degradation during acetaminophen toxicity. Biochem. Pharmacol. 79, 57-66 (2010).
26. Moore, M. et al. The toxicity of acetaminophen and N-acetyl-p-benzoquinone imine in isolated hepatocytes is associated with thiol depletion and increased cytosolic Ca2+. The Journal of biological chemistry 260, 13035-40 (1985).
27. Tiegs, G., Karimi, K., Brune, K. & Arck, P. New problems arising from old drugs: second-generation effects of acetaminophen. Expert Rev Clin Pharmacol 7, 655-62 (2014).
28. Bazan, H. A. et al. A novel pipeline of 2-(benzenesulfonamide)-N-(4-hydroxyphenyl) acetamide analgesics that lack hepatotoxicity and retain antipyresis. European Journal of Medicinal Chemistry 202, 112600 (2020).
29. Santoh, M. et al. Acetaminophen analog N-acetyl-m-aminophenol, but not its reactive metabolite, N-acetyl-p-benzoquinone imine induces CYP3A activity via inhibition of protein degradation. Biochem Biophys Res Commun 486, 639-644 (2017).
30. Bao, Y. et al. Alterations of Cytochrome P450-Mediated Drug Metabolism during Liver Repair and Regeneration after Acetaminophen-Induced Liver Injury in Mice. Drug Metab Dispos 50, 694-703 (2022).
31. Yu, L. et al. Male-specific induction of CYP3A2 in rats by zolmitriptan. J Pharm Pharmacol 60, 1601-1607 (2008).
32. Esteves, F., Rueff, J. & Kranendonk, M. The Central Role of Cytochrome P450 in Xenobiotic Metabolism-A Brief Review on a Fascinating Enzyme Family. J Xenobiot 11, 94-114(2021).
33. McGill, M. R., Williams, C. D., Xie, Y., Ramachandran, A. & Jaeschke, H. Acetaminophen-induced liver injury in rats and mice: comparison of protein adducts, mitochondrial dysfunction, and oxidative stress in the mechanism of toxicity. Toxicol Appl Pharmacol 264, 387-394 (2012).
34. McGill, M. R. & Jaeschke, H. Metabolism and disposition of acetaminophen: recent advances in relation to hepatotoxicity and diagnosis. Pharm Res 30, 2174-2187 (2013).
35. Jaeschke, H. Acetaminophen Hepatotoxicity: Not as Simple as One Might Think! Introductory Comments on the Special Issue-Recent Advances in Acetaminophen Hepatotoxicity. Livers 2, 105-107 (2022).
36. Ramachandran, A. & Jaeschke, H. A mitochondrial journey through acetaminophen hepatotoxicity. Food Chem Toxicol 140, 111282 (2020).
37. Gamal, W. et al. Low-dose acetaminophen induces early disruption of cell-cell tight junctions in human hepatic cells and mouse liver. Sci Rep 7, 37541 (2017).
38. McGill, M. R. et al. Plasma and liver acetaminophen-protein adduct levels in mice after acetaminophen treatment: dose-response, mechanisms, and clinical implications. Toxicol Appl Pharmacol 269, 240-249 (2013).
39. Hestehave, S., Abelson, K. S. P., Brønnum Pedersen, T. & Munro, G. The analgesic efficacy of morphine varies with rat strain and experimental pain model: implications for target validation efforts in pain drug discovery. European Journal of Pain 23, 539-554 (2019).
40. Liao, H.-Y., Hsieh, C.-L., Huang, C.-P. & Lin, Y.-W. Electroacupuncture Attenuates CFA-induced Inflammatory Pain by suppressing Nav1.8 through S100B, TRPV1, Opioid, and Adenosine Pathways in Mice. Sci Rep 7, 42531 (2017).
41. Zhao, L. et al. An analgesic peptide H-20 attenuates chronic pain via the PD-1 pathway with few adverse effects. Proc. Natl. Acad. Sci. U.S.A. 119, e2204114119 (2022).
42. Pahng, A. R., Paulsen, R. I., McGinn, M. A., Edwards, K. N. & Edwards, S.

Neurobiological Correlates of Pain Avoidance-Like Behavior in Morphine-Dependent and Non-Dependent Rats. Neuroscience 366, 1-14 (2017).
43. Barriere, D. A. et al. Paracetamol is a centrally acting analgesic using mechanisms located in the periaqueductal grey. Br J Pharmacol 177, 1773-1792 (2020).
44. Novak, P. 88—Central Autonomic Network. in Neurobiology of Disease (ed. Gilman, S.) 969-977 (Academic Press, 2007). doi:10.1016/B978-012088592-3/50090-6.
45. Hogestatt, E. D. et al. Conversion of acetaminophen to the bioactive N-acylphenolamine AM404 via fatty acid amide hydrolase-dependent arachidonic acid conjugation in the nervous system. The Journal of biological chemistry 280, 31405-12 (2005).
46. Muramatsu, S. et al. Metabolism of AM404 From Acetaminophen at Human Therapeutic Dosages in the Rat Brain. Anesth Pain Med 6, e32873 (2016).
47. Singh, S. & Kaur, M. Mechanism of Action, Kinetics and a Bioactive Metabolites AM404 of Paracetamol. Journal of Clinical Medical Research 01, (2020).
48. Mallet, C. et al. TRPV1 in brain is involved in acetaminophen-induced antinociception. PloS one 5, (2010).
49. Klinger-Gratz, P. P. et al. Acetaminophen Relieves Inflammatory Pain through CB1 Cannabinoid Receptors in the Rostral Ventromedial Medulla. J Neurosci 38, 322-334 (2018).
50. Ashburner, M. et al. Gene Ontology: tool for the unification of biology. Nat Genet 25, 25-29 (2000).
51. Wistrom, E., Chase, R., Smith, P. R. & Campbell, Z. T. A compendium of validated pain genes. WIREs Mech Dis 14, e1570 (2022).
52. Stephens, K. E. et al. Global gene expression and chromatin accessibility of the peripheral nervous system in animal models of persistent pain. J Neuroinflammation 18, 185 (2021).
53. Jin, S. et al. Inference and analysis of cell-cell communication using CellChat. Nat Commun 12, 1088 (2021).
54. Pahng, A. R. & Edwards, S. The Convergent Neuroscience of Affective Pain and Substance Use Disorder. Alcohol Res 41, 14 (2021).
55. Spencer, M. R., Minino, A. M. & Warner, M. Drug Overdose Deaths in the United States, 2001-2021. NCHS Data Brief 1-8 (2022).
56. National Institute on Drug Abuse (NIDA). Opioids: Drug Overdose Death Rates. https://nida.nih.gov/research-topics/trends-statistics/overdose-death-rates (2023).
57. Gorrochategui, E. et al. Paracetamol/acetaminophen hepatotoxicity: new markers for monitoring the elimination of the reactive N-Acetyl-p-benzoquinone imine. http://biorxiv.org/lookup/doi/10.1101/2023.04.28.538718 (2023) doi:10.1101/2023.04.28.538718.
58. Yoon, E., Babar, A., Choudhary, M., Kutner, M. & Pyrsopoulos, N. Acetaminophen-Induced Hepatotoxicity: a Comprehensive Update. J Clin Transl Hepatol 4, 131-142 (2016).
59. Subramanya, S. B. et al. Therapeutic Potential of Plants and Plant Derived Phytochemicals against Acetaminophen-Induced Liver Injury. Int J Mol Sci 19, 3776 (2018).
60. Jaeschke, H. et al. Recommendations for the use of the acetaminophen hepatotoxicity model for mechanistic studies and how to avoid common pitfalls. Acta Pharmaceutica Sinica B 11, 3740-3755 (2021).
61. Research, C. for D. E. and. Acetaminophen. FDA (2023).
62. Research, C. for D. E. and. FDA Drug Safety Communication: Prescription Acetaminophen Products to be Limited to 325 mg Per Dosage Unit; Boxed Warning Will Highlight Potential for Severe Liver Failure. FDA (2019).
63. Piomelli, D. More surprises lying ahead. The endocannabinoids keep us guessing. Neuropharmacology 76 Pt B, 228-234 (2014).
64. Petrosino, S. & Di Marzo, V. The pharmacology of palmitoylethanolamide and first data on the therapeutic efficacy of some of its new formulations. Br J Pharmacol 174, 1349-1365 (2017).
65. Barriere, D. A. et al. Paracetamol is a centrally acting analgesic using mechanisms located in the periaqueductal grey. British Journal of Pharmacology 177, 1773-1792 (2020).
66. Gada, K. & Plant, L. D. Two-pore domain potassium channels: emerging targets for novel analgesic drugs: IUPHAR Review 26. British Journal of Pharmacology 176, 256-266 (2019).
67. Tsantoulas, C. & McMahon, S. B. Opening paths to novel analgesics: the role of potassium channels in chronic pain. Trends in Neurosciences 37, 146-158 (2014).
68. Dowell, D., Ragan, K. R., Jones, C. M., Baldwin, G. T. & Chou, R. CDC Clinical Practice Guideline for Prescribing Opioids for Pain—United States, 2022. MMWR. Recommendations and Reports 71, 1-95 (2023).
69. Thomas, D. & Wessel, C. BIO Industry Analysis. The State of Innovation in Pain and Addiction. 1-24 (www.bio.org/iareports) (2023).
70. Vaccarino, A. L. et al. Synthesis and in vivo evaluation of non-hepatotoxic acetaminophen analogs. Bioorg Med Chem 15, 2206-15 (2007).
71. McGill, M. R. et al. The mechanism underlying acetaminophen-induced hepatotoxicity in humans and mice involves mitochondrial damage and nuclear DNA fragmentation. J Clin Invest 122, 1574-1583 (2012).
72. Ramachandran, A. & Jaeschke, H. Mitochondria in Acetaminophen-Induced Liver Injury and Recovery: A Concise Review. Livers 3, 219-231 (2023).
73. Chow, S.-C. Bioavailability and Bioequivalence in Drug Development. Wiley Interdiscip Rev Comput Stat 6, 304-312 (2014).
74. Reagan-Shaw, S., Nihal, M. & Ahmad, N. Dose translation from animal to human studies revisited. FASEB J 22, 659-661 (2008).
75. Bazan, H. A. et al. A novel pipeline of 2-(benzenesulfonamide)-N-(4-hydroxyphenyl) acetamide analgesics that lack hepatotoxicity and retain antipyresis. Eur J Med Chem 202, 112600 (2020).
76. Vitale, A. & Ricceri, L. The principle of the 3Rs between aspiration and reality. Front Physiol 13, 914939 (2022).
77. Tannenbaum, J. & Bennett, B. T. Russell and Burch's 3Rs then and now: the need for clarity in definition and purpose. J Am Assoc Lab Anim Sci 54, 120-132 (2015).
78. Lewis, D. I. Animal experimentation: implementation and application of the 3Rs. Emerg Top Life Sci 3, 675-679 (2019).
79. Verderio, P. et al. 3Rs Principle and Legislative Decrees to Achieve High Standard of Animal Research. Animals (Basel) 13, 277 (2023).
80. King, W. W., Paramastri, Y. A. & GuillÁ©n, J. Compliance and Regulatory Programs. in Management of Animal Care and Use Programs in Research, Education, and Testing (eds. Weichbrod, R. H., Thompson, G. A. (Heidbrink) & Norton, J. N.) (CRC Press/Taylor & Francis, 2018).
81. Gupta, A. K. et al. Analgesic and Anti-Inflammatory Properties of Gelsolin in Acetic Acid Induced Writhing, Tail Immersion and Carrageenan Induced Paw Edema in Mice. PloS one 10, e0135558 (2015).
82. Jakobsen, J. C., Gluud, C., Wetterslev, J. & Winkel, P. When and how should multiple imputation be used for handling missing data in randomised clinical trials—a practical guide with flowcharts. BMC Med Res Methodol 17, 162 (2017).
83. Stuart, T. et al. Comprehensive Integration of Single-Cell Data. Cell 177, 1888-1902.e21 (2019).
84. Ianevski, A., Giri, A. K. & Aittokallio, T. Fully-automated and ultra-fast cell-type identification using specific marker combinations from single-cell transcriptomic data. Nat Commun 13, 1246 (2022).
85. Wu, T. et al. clusterProfiler 4.0: A universal enrichment tool for interpreting omics data. The Innovation 2, 100141 (2021).
86. Yu, G., Wang, L.-G., Yan, G.-R. & He, Q.-Y. DOSE: an R/Bioconductor package for disease ontology semantic and enrichment analysis. Bioinformatics 31, 608-609 (2015).

While specific examples have been shown and described in detail to illustrate the application of the principles of the present disclosure, it will be understood that the disclosure may be embodied otherwise without departing from such principles. For example, disclosed features may be combined, rearranged, omitted, etc. to produce additional embodiments, while certain disclosed features may sometimes be used to advantage without a corresponding use of other features. Accordingly, Applicant intends to embrace all such alternative, modifications, equivalents, and variations that are within the spirit and scope of the present disclosure. All references, publications and patent documents are herein incorporated by reference.

What is claimed is:

1. A method for ameliorating a pain in a subject in need thereof, the method comprising:
administering a therapeutic amount of a composition comprising SRP-001 or a pharmaceutically acceptable salt thereof, wherein SRP-001 induces analgesia by generating N-arachidonoylphenolamine (AM404) in the midbrain periaqueductal gray (PAG) of the subject, to thereby ameliorate the pain.

2. The method of claim 1, wherein the pain is categorized as acute, chronic, or neuropathic pain.

3. The method of claim 1, wherein the therapeutically effective amount comprises a dose of about 10 μM to about 10 mM of the composition is administered to the subject.

4. The method of claim 1, wherein the composition is administered in a single dose.

5. The method of claim 1, wherein the composition is administered at intervals of about 4 hours, 12 hours, or 24 hours.

6. The method of claim 1, wherein the composition is administered orally, parentally, transdermally, topically, or nasally.

7. The method of claim 1, wherein the composition further comprises one or more pharmaceutically acceptable excipients, carriers, or a combination thereof.

8. The method of claim 1, wherein the SRP-001 is present in an amount selected from the group consisting of from about 99.5% to about 0.001%, from about 95% to about 0.1%, and from about 90% to about 0.5%, by weight, based on the total combined weight of the SRP-100 and at least one wetting agent, not including other excipients.

9. The method of claim 8, wherein the at least one wetting agent is present in an amount selected from the group consisting of from about 0.01% to about 99.5% by weight, from about 0.1% to about 95% by weight, and from about 0.5% to about 90% by weight, and from about 0.5% to about 2%, based on the total combined dry weight of SRP-100 and at least one wetting agent, not including other excipients.

10. The method of claim 8, wherein the at least one wetting agent is present in an amount selected from the group consisting of from about less than 10%, less than 5%, less than 2% and less than 1%, based on the weight of the nanoparticulate composition.

11. The method of claim 8, wherein the at least one wetting agent is selected from the group consisting of a bile salt and an alkali salt of a bile acid.

12. The method of claim 11, wherein the bile salt is selected from the group consisting of sodium, potassium, lithium, calcium, arginine, lysine and the ammonium salt of a bile acid.

13. The method of claim 11, comprising a salt of a bile acid selected from the group consisting of cholic acid, glycocholic acid, taurocholic acid, deoxycholic acid, glyco- or taurodeoxycholic acid, chenodeoxycholic acid and glyco- or taurochenoxydeoxycholic acid.

14. The method of claim 13, further comprising a secondary wetting agent selected from the group consisting of polyvinyl alcohol, polyvinylpyrrolidone, polyethylene glycol, and cellulose derivatives.

15. The method of claim 1, wherein the composition is administered in the form of a pill, tablet, capsule, cream, spray, lotion, a suspension or aqueous solution.

16. The method of claim 1, wherein the composition is a nanoparticulate particle having an average particle size of less than about 500 nm or less than about 300 nm.

17. The method of claim 1, wherein the composition exhibits analgesia, antipyresis, or a combination thereof.

18. The method of claim 1, wherein the composition reduces the risk of hepatotoxicity by at least about 20% compared to acetaminophen (ApAP).

19. The method of claim 1, wherein the administration does not generate the hepatotoxic metabolite NAPQI.

20. The method of claim 1, wherein the specific neuronal target of the composition is one or more of CB1, FAAH or TRPV1, which underlies the analgesia; or wherein SRP-001 has a half-life in said subject of about 4.9 to about 9.8 hours; or wherein in a direct comparison of SRP-001 vs. ApAP at equimolar doses reveals no mortality for SRP-001, whereas ApAP shows a dose-dependent increase in mortality.
